SEQUENCE LISTING (71 sequences)

SEQ ID NO: 1 - DNA, 6540 bp, Homo sapiens
  CDS (131)..(5140)
  sig_peptide (131)..(211)
aggtctccgcttggagccgccgcacccgggacggtgcgtagcgctggaagtccggccttccgagagctagctgtccgccgcggcccccgcacgccgggcagccgtccctcgccgcctcgggcgcgccaccatggggccccggctcagcgtctggctgctgctgctgcccgccgcccttctgctccacgaggagcacagccgggccgctgcgaagggtggctgtgctggctctggctgtggcaaatgtgactgccatggagtgaagggacaaaagggtgaaagaggcctcccggggttacaaggtgtcattgggtttcctggaatgcaaggacctgaggggccacagggaccaccaggacaaaagggtgatactggagaaccaggactacctggaacaaaagggacaagaggacctccgggagcatctggctaccctggaaacccaggacttcccggaattcctggccaagacggcccgccaggccccccaggtattccaggatgcaatggcacaaagggggagagagggccgctcgggcctcctggcttgcctggtttcgctggaaatcccggaccaccaggcttaccagggatgaagggtgatccaggtgagatacttggccatgtgcccgggatgctgttgaaaggtgaaagaggatttcccggaatcccagggactccaggcccaccaggactgccagggcttcaaggtcctgttgggcctccaggatttaccggaccaccaggtcccccaggccctcccggccctccaggtgaaaagggacaaatgggcttaagttttcaaggaccaaaaggtgacaagggtgaccaaggggtcagtgggcctccaggagtaccaggacaagctcaagttcaagaaaaaggagacttcgccaccaagggagaaaagggccaaaaaggtgaacctggatttcaggggatgccaggggtcggagagaaaggtgaacccggaaaaccaggacccagaggcaaacccggaaaagatggtgacaaaggggaaaaagggagtcccggttttcctggtgaacccgggtacccaggactcataggccgccagggcccgcagggagaaaagggtgaagcaggtcctcctggcccacctggaattgttataggcacaggacctttgggagaaaaaggagagaggggctaccctggaactccggggccaagaggagagccaggcccaaaaggtttcccaggactaccaggccaacccggacctccaggcctccctgtacctgggcaggctggtgcccctggcttccctggtgaaagaggagaaaaaggtgaccgaggatttcctggtacatctctgccaggaccaagtggaagagatgggctcccgggtcctcctggttcccctgggccccctgggcagcctggctacacaaatggaattgtggaatgtcagcccggacctccaggtgaccagggtcctcctggaattccagggcagccaggatttataggcgaaattggagagaaaggtcaaaaaggagagagttgcctcatctgtgatatagacggatatcgggggcctcccgggccacagggacccccgggagaaataggtttcccagggcagccaggggccaagggcgacagaggtttgcctggcagagatggtgttgcaggagtgccaggccctcaaggtacaccagggctgataggccagccaggagccaagggggagcctggtgagttttatttcgacttgcggctcaaaggtgacaaaggagacccaggctttccaggacagcccggcatgccagggagagcgggttctcctggaagagatggccatccgggtcttcctggccccaagggctcgccgggttctgtaggattgaaaggagagcgtggcccccctggaggagttggattcccaggcagtcgtggtgacaccggcccccctgggcctccaggatatggtcctgctggtcccattggtgacaaaggacaagcaggctttcctggaggccctggatccccaggcctgccaggtccaaagggtgaaccaggaaaaattgttcctttaccaggcccccctggagcagaaggactgccggggtccccaggcttcccaggtccccaaggagaccgaggctttcccggaaccccaggaaggccaggcctgccaggagagaagggcgctgtgggccagccaggcattggatttccagggccccccggccccaaaggtgttgacggcttacctggagacatggggccaccggggactccaggtcgcccgggatttaatggcttacctgggaacccaggtgtgcagggccagaagggagagcctggagttggtctaccgggactcaaaggtttgccaggtcttcccggcattcctggcacacccggggagaaggggagcattggggtaccaggcgttcctggagaacatggagcgatcggaccccctgggcttcaggggatcagaggtgaaccgggacctcctggattgccaggctccgtggggtctccaggagttccaggaataggcccccctggagctaggggtccccctggaggacagggaccaccggggttgtcaggccctcctggaataaaaggagagaagggtttccccggattccctggactggacatgccgggccctaaaggagataaaggggctcaaggactccctggcataacgggacagtcggggctccctggccttcctggacagcagggggctcctgggattcctgggtttccaggttccaagggagaaatgggcgtcatggggacccccgggcagccgggctcaccaggaccagtgggtgctcctggattaccgggtgaaaaaggggaccatggctttccgggctcctcaggacccaggggagaccctggcttgaaaggtgataagggggatgtcggtctccctggcaagcctggctccatggataaggtggacatgggcagcatgaagggccagaaaggagaccaaggagagaaaggacaaattggaccaattggtgagaagggatcccgaggagaccctgggaccccaggagtgcctggaaaggacgggcaggcaggacagcctgggcagccaggacctaaaggtgatccaggtataagtggaaccccaggtgctccaggacttccgggaccaaaaggatctgttggtggaatgggcttgccaggaacacctggagagaaaggtgtgcctggcatccctggcccacaaggttcacctggcttacctggagacaaaggtgcaaaaggagagaaagggcaggcaggcccacctggcataggcatcccagggctgcgaggtgaaaagggagatcaagggatagcgggtttcccaggaagccctggagagaagggagaaaaaggaagcattgggatcccaggaatgccagggtccccaggccttaaagggtctcccgggagtgttggctatccaggaagtcctgggctacctggagaaaaaggtgacaaaggcctcccaggattggatggcatccctggtgtcaaaggagaagcaggtcttcctgggactcctggccccacaggcccagctggccagaaaggggagccaggcagtgatggaatcccggggtcagcaggagagaagggtgaaccaggtctaccaggaagaggattcccagggtttccaggggccaaaggagacaaaggttcaaagggtgaggtgggtttcccaggattagccgggagcccaggaattcctggatccaaaggagagcaaggattcatgggtcctccggggccccagggacagccggggttaccgggatccccaggccatgccacggaggggcccaaaggagaccgcggacctcagggccagcctggcctgccaggacttccgggacccatggggcctccagggcttcctgggattgatggagttaaaggtgacaaaggaaatccaggctggccaggagcacccggtgtcccagggcccaagggagaccctggattccagggcatgcctggtattggtggctctccaggaatcacaggctctaagggtgatatggggcctccaggagttccaggatttcaaggtccaaaaggtcttcctggcctccagggaattaaaggtgatcaaggcgatcaaggcgtcccgggagctaaaggtctcccgggtcctcctggccccccaggtccttacgacatcatcaaaggggagcccgggctccctggtcctgagggccccccagggctgaaagggcttcagggactgccaggcccgaaaggccagcaaggtgttacaggattggtgggtatacctggacctccaggtattcctgggtttgacggtgcccctggccagaaaggagagatgggacctgccgggcctactggtccaagaggatttccaggtccaccaggccccgatgggttgccaggatccatggggcccccaggcaccccatctgttgatcacggcttccttgtgaccaggcatagtcaaacaatagatgacccacagtgtccttctgggaccaaaattctttaccacgggtactctttgctctacgtgcaaggcaatgaacgggcccatggccaggacttgggcacggccggcagctgcctgcgcaagttcagcacaatgcccttcctgttctgcaatattaacaacgtgtgcaactttgcatcacgaaatgactactcgtactggctgtccacccctgagcccatgcccatgtcaatggcacccatcacgggggaaaacataagaccatttattagtaggtgtgctgtgtgtgaggcgcctgccatggtgatggccgtgcacagccagaccattcagatcccaccgtgccccagcgggtggtcctcgctgtggatcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataatgaagcctgactcagctaatgtcacaacatggtgctacttcttcttctttttgttaacagcaacgaaccctagaaatatatcctgtgtacctcactgtccaatatgaaaaccgtaaagtgccttataggaatttgcgtaactaacacaccctgcttcattgacctctacttgctgaaggagaaaaagacagcgataagctttcaatagtggcataccaaatggcacttttgatgaaataaaatatcaatattttctgcaatccaatgcactgatgtgtgaagtgagaactccatcagaaaaccaaagggtgctaggaggtgtgggtgccttccatactgtttgcccattttcattcttgtattataattaattttctacccccagagataaatgtttgtttatatcactgtctagctgtttcaaaatttaggtcccttggtctgtacaaataatagcaatgtaaaaatggttttttgaacctccaaatggaattacagactcagtagccatatcttccaaccccccagtataaatttctgtctttctgctatgtgtggtactttgcagctgcttttgcagaaatcacaattttcctgtggaataaagatggtccaaaaatagtcaaaaattaaatatatatatatattagtaatttatatagatgtcagcaattaggcagatcaaggtttagtttaacttccactgttaaaataaagcttacatagttttcttcctttgaaagactgtgctgtcctttaacataggtttttaaagactaggatattgaatgtgaaacatccgttttcattgttcacttctaaaccaaaaattatgtgttgccaaaaccaaacccaggttcatgaatatggtgtctattatagtgaaacatgtactttgagcttattgtttttattctgtattaaatattttcagggttttaaacactaatcacaaactgaatgacttgacttcaaaagcaacaaccttaaaggccgtcatttcattagtattcctcattctgcatcctggcttgaaaaacagctctgttgaatcacagtatcagtattttcacacgtaagcacattcgggccatttccgtggtttctcatgagctgtgttcacagacctcagcagggcatcgcatggaccgcaggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgcatacctta

SEQ ID NO: 2 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMPGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 3 - DNA, 6446 bp, Homo sapiens
  CDS (290)..(5428)
  sig_peptide (290)..(388)
gagtgtggctgcagtgcgccgggacaccagggctccgcgctccgcactcaagaggctcccgcgtcccaacccctcgcgcccgcgcgttcgcggatccaggccgaggaccgaaaggggccgcccgagcccccggggccggcgcccagagagcccagcaaggccggccgccctgccggtgtgccgccggcgggtgcttctggaagggccaatgcgttcgggcagcagcccctgaagccgagcccgaggctaagtgggactgaccggggcccagagtggacgaaccgccagcatggggagagaccagcgcgcggtggccggccctgccctacggcggtggctgctgctggggacagtgaccgtggggttcctcgcccagagcgtcttggcgggtgtgaagaagtttgatgtgccgtgtggaggaagagattgcagtgggggctgccagtgctaccctgagaaaggtggacgtggtcagcctgggccagtgggcccccaggggtacaatgggccaccaggattacaaggattcccgggactgcagggacgtaaaggagacaagggtgaaaggggagcccccggagtaacgggacccaagggcgacgtgggagcaagaggcgtttctggattccctggtgccgatggaattcctggacacccggggcaaggtgggcccaggggaaggccgggctacgatggctgcaacggaacccagggagactcaggtccacaggggccccccggctctgaggggttcaccgggcctcccgggccccaaggaccaaaagggcagaaaggtgagccttatgcactgcctaaagaggagcgcgacagatatcggggtgaacctggagagcctggattggtcggtttccagggacctcccggccgccctgggcatgtgggacagatgggtccagttggagctccagggagaccaggaccacctggaccccctggaccaaaaggacagcaaggcaacagaggacttggtttctacggagttaagggtgaaaagggtgacgtagggcagccgggacccaacgggattccatcagacaccctccaccccatcatcgcgcccacaggagtcaccttccacccagatcagtacaagggtgaaaaaggcagtgagggggaaccaggaataagaggcatttccttgaagggagaagaaggaatcatgggctttcctggactgaggggttaccctggcttgagtggtgaaaaaggatcaccaggacagaagggaagccgaggcctggatggctatcaagggcctgatggaccccggggacccaagggagaagccggagacccagggccccctggactacctgcctactcccctcacccttccctagcaaaaggtgccagaggtgacccgggattcccaggggcccaaggggagccaggaagccagggtgagccaggagacccgggcctcccaggtccccctggcctctccatcggagatggagatcagaggagaggcctgccgggtgagatgggacccaagggcttcatcggagaccccggcatccctgcgctctacgggggcccacctggacctgatggaaagcgagggcctccaggaccccccgggctccctggaccacctggacctgatggcttcctgtttgggctgaaaggagcaaaaggaagagcaggcttccctgggcttcccggctcccctggagcccgcggaccaaaggggtggaaaggtgacgctggggaatgcagatgtacagaaggcgacgaagctatcaaaggtcttccgggactgccaggacccaagggcttcgcaggcatcaacggggagccggggaggaaaggggacagaggagaccccggccaacacggcctccctgggttcccagggctcaagggagtgcctggcaacattggtgctcccggacccaaaggagcaaaaggagattccagaacaatcacaaccaaaggtgagcggggacagcccggcgtcccaggtgtgcccgggatgaaaggtgacgatggcagcccaggccgcgatgggctcgatggattccccggcctcccaggccctcccggtgatggcatcaagggccctccaggggacccaggctatccaggaatacctggaacgaagggtactccaggagaaatgggccccccaggactgggccttcccggcctcaaaggccaacgtggtttccctggagacgccggcttacctggaccaccaggcttcctgggccctcctggccccgcagggaccccaggacaaatagattgtgacacagatgtgaaaagggccgttggaggtgacagacaggaggccatccagccaggttgcataggagggcccaagggattgccaggcctgccaggacccccaggccccacaggtgccaaaggcctccgaggaatcccaggcttcgcaggagctgatggaggaccagggcccaggggcttgccaggagacgcaggtcgtgaagggttcccaggacccccagggttcataggaccccgaggatccaaaggtgcagtgggcctccctggcccagatggatccccaggtcccatcggcctgccagggccagatgggccccctggggaaaggggcctccctggagaagtcctgggagctcagcccgggccacggggagatgctggtgtgcctggacagcctgggcttaaaggccttcccggagacagaggcccccctggattcagaggaagccaagggatgcctgggatgccagggctgaagggccagccaggcctcccaggaccttccggccagccaggcctgtatgggcctccaggactgcatggattcccaggagctcctggccaagaggggcccttggggctgccaggaatcccaggccgtgaaggtctgcctggtgatagaggggaccctggggacacaggcgctcctggccctgtgggcatgaaaggtctctctggtgacagaggagatgctggcttcacaggggagcaaggccatccaggaagccctggatttaaaggaattgatggaatgcctgggacccccgggctaaaaggagatagaggctcacctgggatggatggtttccaaggcatgcctggactcaaagggagacccgggtttccagggagcaaaggcgaggctggatttttcggaatacccggtctgaagggtctggctggtgagccaggttttaaaggcagccgaggggaccctgggcccccaggaccacctcctgtcatcctgccaggaatgaaagacattaaaggagagaaaggagatgaagggcctatggggctgaaaggatacctgggcgcaaaaggtatccaaggaatgccaggcatcccagggctgtcaggaatccctgggctgcctgggaggcccggccacatcaaaggagtcaagggagacatcggagtccccggcatccccggtttgccaggattccctggggtggctggcccccctggaattacgggattcccaggattcataggaagccggggtgacaaaggtgccccagggagagcaggcctgtatggcgagattggcgcgactggtgatttcggtgacatcggggacactataaatttaccaggaagaccaggcctgaagggggagcggggcaccactggaataccaggtctgaagggattctttggagagaagggaacagaaggtgacatcggcttccctgggataacaggcgtgactggagtccaaggccctcctggacttaaaggacaaacaggctttccagggctgactgggcctccagggtcgcagggagagctggggcggattggactgcctggtggcaaaggagatgatggctggccgggagctccgggcttaccaggttttccgggactccgtgggatccgcggcttacacggcttgccaggcaccaagggctttccaggatccccaggttctgacatccacggagacccaggcttcccaggccctcctggggaaagaggtgacccaggagaggccaacacccttccaggccctgtgggagtcccaggacagaaaggagaccaaggagctccaggggaacgaggcccacctgggagcccaggacttcaggggttccctggtatcacacccccttccaacatctctggggcacctggtgacaaaggggcgccagggatatttggcctgaaaggttatcggggcccaccagggccaccaggttctgctgctcttcctggaagcaaaggtgacacagggaacccaggagctccaggaaccccagggaccaaaggatgggccggggactccgggccccagggcaggcctggtgtgtttggtctcccaggagaaaaagggcccaggggtgaacaaggcttcatggggaacactggacccactggggcggtgggcgacagaggccccaagggacccaagggagacccaggattccctggtgcccccgggactgtgggagcccccgggattgcaggaatcccccagaagattgccgtccaaccagggacagtgggtccccaggggaggcgaggcccccctggggcaccgggggagatggggccccagggcccccccggagaaccaggtttccgtggggctccagggaaagctgggccccaaggaagaggtggtgtgtctgctgttcccggcttccggggagatgaaggacccataggccaccaggggccgattggccaagaaggtgcaccaggccgtccagggagcccgggcctgccgggtatgccaggccgcagcgtcagcatcggctacctcctggtgaagcacagccagacggaccaggagcccatgtgcccagtgggcatgaacaaactctggagtggatacagcctgctgtacttcgagggccaggagaaggcgcacaaccaggacctggggctggcgggctcctgcctggcgcggttcagcaccatgcccttcctgtactgcaaccctggtgatgtctgctactatgccagccggaacgacaagtcctactggctctctaccactgcgccgctgcccatgatgcccgtggccgaggacgagatcaagccctacatcagccgctgttctgtgtgtgaggccccggccatcgccatcgcggtccacagtcaggatgtctccatcccacactgcccagctgggtggcggagtttgtggatcggatattccttcctcatgcacacggcggcgggagacgaaggcggtggccaatcactggtgtcaccgggcagctgtctagaggacttccgcgccacaccattcatcgaatgcaatggaggccgcggcacctgccactactacgccaacaagtacagcttctggctgaccaccattcccgagcagagcttccagggctcgccctccgccgacacgctcaaggccggcctcatccgcacacacatcagccgctgccaggtgtgcatgaagaacctgtgagccggcgcgtgccaggaagggccattttggtgcttattcttaacttattacctcaggtgccaacccaaaaattggttttatttttttcttaaaaaaaaaaaagtctaccaaaggaatttgcatccagcagcagcacttagacctgccagccactgtcaccgagcgggtgcaagcactcggggtccctggagggcaagccctgcccacagaaagccaggagcagccctggcccccatcagccctgctagacgcaccgcctgaaggcacagctaaccacttcgcacacacccatgtaaccactgcactttccaatgccacagacaactcacattgttcaactcccttctcggggtgggacagacgagacaacagcacacaggcagccagccgtggccagaggctcgaggggctcagggcctcaggcacccgtccccacacgagggccccgtgggtgggcctggccctgctttctacgccaatgttatgccagctccatgttctcccaaataccgttgatgtgaattattttaaaggcaaaaccgtgctctttattttaaaaaacactgataatcacactgcggtaggtcattcttttgccacatccctatagaccactgggtttggcaaaactcaggcagaagtggagacctttctagacatcattgtcagccttgctacttgaaggtacaccccatagggtcggaggtgctgtccccactgccccacgttgtccctgagatttaacccctccactgctgggggtgagctgtactcttctgactgccccctcctgtgtaacgactacaaaataaaacttggttctgaatatttttaaaccccgagttgttgaccgccttaatctcgtgtccatagagcaaaacgtctgctcagatggatgcgaggcacagcgtccgcccacgctgctgtttttaatccatctcagtagagttgaacccattcgtggtattacagccatttctcggggaatgtgtttgtttataactcactaatgcttaca

SEQ ID NO: 4 - protein, 1712 aa, Homo sapiens
MGRDQRAVAGPALRRWLLLGTVTVGFLAQSVLAGVKKFDVPCGGRDCSGGCQCYPEKGGRGQPGPVGPQGYNGPPGLQGFPGLQGRKGDKGERGAPGVTGPKGDVGARGVSGFPGADGIPGHPGQGGPRGRPGYDGCNGTQGDSGPQGPPGSEGFTGPPGPQGPKGQKGEPYALPKEERDRYRGEPGEPGLVGFQGPPGRPGHVGQMGPVGAPGRPGPPGPPGPKGQQGNRGLGFYGVKGEKGDVGQPGPNGIPSDTLHPIIAPTGVTFHPDQYKGEKGSEGEPGIRGISLKGEEGIMGFPGLRGYPGLSGEKGSPGQKGSRGLDGYQGPDGPRGPKGEAGDPGPPGLPAYSPHPSLAKGARGDPGFPGAQGEPGSQGEPGDPGLPGPPGLSIGDGDQRRGLPGEMGPKGFIGDPGIPALYGGPPGPDGKRGPPGPPGLPGPPGPDGFLFGLKGAKGRAGFPGLPGSPGARGPKGWKGDAGECRCTEGDEAIKGLPGLPGPKGFAGINGEPGRKGDRGDPGQHGLPGFPGLKGVPGNIGAPGPKGAKGDSRTITTKGERGQPGVPGVPGMKGDDGSPGRDGLDGFPGLPGPPGDGIKGPPGDPGYPGIPGTKGTPGEMGPPGLGLPGLKGQRGFPGDAGLPGPPGFLGPPGPAGTPGQIDCDTDVKRAVGGDRQEAIQPGCIGGPKGLPGLPGPPGPTGAKGLRGIPGFAGADGGPGPRGLPGDAGREGFPGPPGFIGPRGSKGAVGLPGPDGSPGPIGLPGPDGPPGERGLPGEVLGAQPGPRGDAGVPGQPGLKGLPGDRGPPGFRGSQGMPGMPGLKGQPGLPGPSGQPGLYGPPGLHGFPGAPGQEGPLGLPGIPGREGLPGDRGDPGDTGAPGPVGMKGLSGDRGDAGFTGEQGHPGSPGFKGIDGMPGTPGLKGDRGSPGMDGFQGMPGLKGRPGFPGSKGEAGFFGIPGLKGLAGEPGFKGSRGDPGPPGPPPVILPGMKDIKGEKGDEGPMGLKGYLGAKGIQGMPGIPGLSGIPGLPGRPGHIKGVKGDIGVPGIPGLPGFPGVAGPPGITGFPGFIGSRGDKGAPGRAGLYGEIGATGDFGDIGDTINLPGRPGLKGERGTTGIPGLKGFFGEKGTEGDIGFPGITGVTGVQGPPGLKGQTGFPGLTGPPGSQGELGRIGLPGGKGDDGWPGAPGLPGFPGLRGIRGLHGLPGTKGFPGSPGSDIHGDPGFPGPPGERGDPGEANTLPGPVGVPGQKGDQGAPGERGPPGSPGLQGFPGITPPSNISGAPGDKGAPGIFGLKGYRGPPGPPGSAALPGSKGDTGNPGAPGTPGTKGWAGDSGPQGRPGVFGLPGEKGPRGEQGFMGNTGPTGAVGDRGPKGPKGDPGFPGAPGTVGAPGIAGIPQKIAVQPGTVGPQGRRGPPGAPGEMGPQGPPGEPGFRGAPGKAGPQGRGGVSAVPGFRGDEGPIGHQGPIGQEGAPGRPGSPGLPGMPGRSVSIGYLLVKHSQTDQEPMCPVGMNKLWSGYSLLYFEGQEKAHNQDLGLAGSCLARFSTMPFLYCNPGDVCYYASRNDKSYWLSTTAPLPMMPVAEDEIKPYISRCSVCEAPAIAIAVHSQDVSIPHCPAGWRSLWIGYSFLMHTAAGDEGGGQSLVSPGSCLEDFRATPFIECNGGRGTCHYYANKYSFWLTTIPEQSFQGSPSADTLKAGLIRTHISRCQVCMKNL

SEQ ID NO: 5 - DNA, 8114 bp, Homo sapiens
  CDS (163)..(5175)
  sig_peptide (163)..(246)
gggagggacgaaccgcgcgaccgagccctacaaaacccgccccggccgagtggcgaggcgagctttccagccgggctcccagagccgcgctgcgcaggagacgcggtggcctgagagcctgagggtccccggactcgcccaggctctgagcgcgcgcccaccatgagcgcccggaccgcccccaggccgcaggtgctcctgctgccgctcctgctggtgctcctggcggcggcgcccgcagccagcaagggttgtgtctgtaaagacaaaggccagtgcttctgtgacggggccaaaggggagaagggggagaagggctttcctggaccccccggttctcctggccagaaaggattcacaggtcctgaaggcttgcctggaccgcagggacccaagggctttccaggacttccaggactcacgggttccaaaggtgtaaggggaataagtggattgccaggattttctggttctcctggacttccaggcaccccaggcaataccgggccttacggacttgtcggtgtaccaggatgcagtggttctaagggtgagcaggggtttccaggactcccagggacactgggctacccagggatcccgggtgctgctggtttgaaaggacaaaagggtgctcctgctaaagaagaagatatagaacttgatgcaaaaggcgaccccgggttgccaggggctccaggaccccagggtttgccaggccctccaggttttcctgggcctgttggcccacctggtcctccgggattctttggctttccaggagccatgggacctagaggacctaagggtcacatgggtgaaagagtgataggacataaaggagagcggggtgtgaaagggttaacaggacccccgggaccaccaggaacagttattgtgaccctaactggcccagataacagaacggacctcaagggggaaaagggagacaagggagcaatgggcgagcctggacctcctggaccctcaggactgcctggagaatcatatggatctgaaaagggtgctcctggagaccctggcctgcagggaaaacccggaaaagatggtgttcctggcttccctggaagtgagggagtcaagggcaacaggggtttccctgggttaatgggtgaagatggcattaagggacagaaaggggacattggccctccaggatttcgtggtccaacagaatattatgacacataccaggaaaagggagatgaaggcactccaggcccaccagggcccagaggagctcgtggcccacaaggtcccagtggtccccccggagttcctggaagtcctggatcatcaaggcctggcctcagaggagcccctggatggccaggcctgaaaggaagtaaaggggaacgaggccgcccaggaaaggatgccatggggactcctgggtccccaggttgtgctggttcaccaggtcttccaggatcaccgggacctccaggaccgccaggtgacatcgtttttcgcaagggtccacctggagatcacggactgccaggctatctagggtctccaggaatcccaggagttgatgggcccaaaggagaaccaggcctcctgtgtacacagtgcccttatatcccagggcctcccggtctcccaggattgccagggttacatggtgtaaaaggaatcccaggaagacaaggcgcagctggcttgaaaggaagcccagggtccccaggaaatacaggtcttccaggatttccaggtttcccaggtgcccagggtgacccaggacttaaaggagaaaaaggtgaaacacttcagcctgaggggcaagtgggtgtcccaggtgacccggggctcagaggccaacctgggagaaagggcttggatggaattcctggaactccgggagtgaaaggattaccaggacctaaaggcgaactggctctgagtggtgagaaaggggaccaaggtcctccaggggatcctggctcccctgggtccccaggacctgcaggaccagctggaccacctggctacggaccccaaggagaacctggtctccagggcacgcaaggagttcctggagcccccggaccacccggagaagccggccctaggggagagctcagtgtttcaacaccagttccaggcccaccaggacctccagggccccctggccatcctggcccccaaggtccacctggtatccctggatccctggggaaatgtggagatcctggtcttccagggcctgatggtgaaccaggaattccaggaattggatttcctgggcctcctggacctaagggagaccaaggttttccaggtacaaaaggatcactgggttgtcctggaaaaatgggagagcctgggttacctggaaagccaggcctcccaggagccaagggagaaccagcagtagccatgcctggaggaccaggaacaccaggttttccaggagaaagaggcaattctggggaacatggagaaattggactccctggacttccaggtctccctggaactccaggaaatgaagggcttgatggaccacgaggagatccagggcagcctggaccacctggagaacaaggacccccaggaaggtgcatagagggtcccaggggagcccaaggacttccaggcttaaatggattgaaagggcaacaaggcagaagaggtaaaacggggccaaagggagacccaggaattccaggcttggatagatcaggatttcctggagaaactggatcaccaggaattccaggtcatcaaggtgaaatgggaccactgggtcaaagaggatatccaggaaatccgggaattttagggccaccaggtgaagatggagtgattgggatgatgggctttcctggagccattggccctccagggccccctgggaacccaggcacaccagggcagagggggagccctggaattccaggagtaaagggccagagaggaaccccaggagccaagggggaacaaggagataaaggaaatcccgggccttcagagatatcccacgtaataggggacaaaggagaaccaggtctcaaaggattcgcaggaaatccaggtgagaaaggaaacagaggcgttccagggatgccaggtttaaagggcctcaaaggactacccggaccagcaggaccaccaggccccagaggagatttgggcagcactgggaatcctggagaaccaggactgcgtggtataccaggaagcatggggaacatgggcatgccaggttctaaaggaaaaaggggaactttgggattcccaggtcgagcaggaagaccaggcctcccaggtattcatggtctccagggagataagggagagccaggttattcagaaggtacaaggccaggaccaccgggaccaacgggggatccaggactgccgggtgatatgggaaagaaaggagaaatggggcaacctggcccacctggacatttggggcctgctggacctgagggagcccctggaagtcctggaagtcctggcctcccaggaaagccaggtcctcatggtgatttgggttttaaaggaatcaaaggcctcctgggccctccaggaatcagaggccctccaggtcttccaggatttccaggatctcctggaccaatgggtataagaggtgaccaaggacgtgatggaattcctggtccagccggagaaaagggagaaacgggtttattgagggcccctccaggcccaagagggaaccctggtgctcaaggagccaaaggagacaggggagccccaggttttcctggcctcccgggcagaaaaggggccatgggagatgctggacctcgaggacccacaggcatagaaggattcccagggccaccaggtctgcccggtgcaattatccctggccagacaggaaatcgtggtccaccaggctcaagaggaagcccaggtgcgcctggtccccctggacctccagggagtcatgtaataggcataaaaggagacaaagggtctatgggccaccctggcccaaaaggtccacctggaactgcaggagacatgggaccaccaggtcgtctgggagcaccaggtactccaggtcttccaggacccagaggtgatcctggattccaggggtttccaggcgtgaaaggagaaaagggtaatcctggatttctaggatccattggacctccaggaccaattgggccaaaaggaccacctggtgtacgtggagaccctggcacacttaagattatctcccttccaggaagcccagggccacctggcacacctggagaaccagggatgcagggagaacctgggccaccagggccacctggaaacctaggaccctgtgggccaagaggtaagccaggcaaggatggaaaaccaggaactcctggaccagctggagaaaaaggcaacaaaggttctaaaggagagccaggaccagctggatcagatggattgccaggtttgaaaggaaaacgtggagacagtggatcacctgcaacctggacaacgagaggctttgtcttcacccgacacagtcaaaccacagcaattccttcatgtccagaggggacagtgccactctacagtgggttttcttttctttttgtacaaggaaatcaacgagcccacggacaagaccttggaactcttggcagctgcctgcagcgatttaccacaatgccattcttattctgcaatgtcaatgatgtatgtaattttgcatctcgaaatgattattcatactggctgtcaacaccagctctgatgccaatgaacatggctcccattactggcagagcccttgagccttatataagcagatgcactgtttgtgaaggtcctgcgatcgccatagccgttcacagccaaaccactgacattcctccatgtcctcacggctggatttctctctggaaaggattttcattcatcatgttcacaagtgcaggttctgagggcaccgggcaagcactggcctcccctggctcctgcctggaagaattccgagccagcccatttctagaatgtcatggaagaggaacgtgcaactactattcaaattcctacagtttctggctggcttcattaaacccagaaagaatgttcagaaagcctattccatcaactgtgaaagctggggaattagaaaaaataataagtcgctgtcaggtgtgcatgaagaaaagacactgaagctaaaaaagacagcagaactgctatttttcatcctaaagaacaaagtaatgacagaacatgctgttatttaggtatttttctttaaccaaacaatattgctccatgatgacttagtacaaagtttcaatttgtttccccacaaaacaaagcaattctttcaagtcagttctgtgatctgggtctctaatctgtgctgtttcaaagttctctgtggcaaagcagcaactattcacaaaatatcaccaaaaacctattccacttacatccaaggcactgtcactacggtgattgtatgaagtttgaatgctgcaagttatgaaatatttggcccgctggattcccacatttgtcttctttctgtctttaagactcagggaggctaaatcagtgtttgattgccccgccaacccttcctgaaacttcagaccctgggtaggggaagagaagggggcatgtggtatcctggagcattgtgtatagaactggattttcagacctgctgaggaccgtaaggcctgatggaacacagaactgaactgaggttcatggattttccaggactgtttcaaacatgcccattactaacggcaaaagggggattccctgatggaaccataatacccttggaaatactgtatggttttgttttgttttgttggtttttaaagatttttgtttgtttattgaattcatttcactgtagctctaaaatctgcttgtattccaagcatataaaattttcccccttagtgaattagttttaaaatgatattgttatatacatactatgaaatatgtataactttaacttctgttttaccagcatacccacacaaataacaagaatactacttatgaaatgtgcactttatcctcattccataaatgtcggtgcataccttatgtaagggagcagttcaataatccatgaaagaacttaaggcatttgttggtttatcagactcggaatctattttctcattgctctgaatatgtcatcactctaggttttacagatttattcctttgttacttctctaattcttcctttgtaaaaaaaaaaaaaagcaacactttttatgttatatgttgttcttacaaaccatactgaaagagtccattgtttaaaaatcttaatgtatcaaactgtataacttggccgctgtatgtcttaaaacctgcttttcaatgtgttgatacattcccaaggttacttaattcaacttaactatcatcttattcagcaccaagcatgtcccaggcactgtactaacctacagagatgctaagagaaaaaaaagacttgtttctgatctaatatcccagaaaaagtaactcattgctctgttaataatctcacatatacaagtagcttccctcccctctagttttttcttccttttcactgctgttatatttcatcatgataattcagcaggcccaagtaaaggttaaaaataaggtctatgcctagggaaactcagggcttctagtttctcttagaaaagctaagagaagataaggtctgaataatagcagaaaaaccaacatctacaaaacattaaactagtgttatacttgatgataacactatttgatgagtcttagagtccagacacaaagagacaaagctttgaagatgctttttgatctacctaggtggagttggtggtgctgatatttaaattcaggctactgcttcaatctcaattgctttgtaagtgaaaaacatgacccagaggacagcacagactatggccatggctcacatggtttacatccttcactgctcacgtgtttgctgtcaagccatttttacatctaaactaagatgtgcagcatttcacttatttagattcacttaacaaacaaatttttctgctttaaaaatgtcttattgtcccaagtgtactatagcggcatatagagctagctaatctctacaaaccctctgtaggccagtagttctcaaagtgtggtctctggaagagcagtatcagcatcatctgggaacttgtcacagatgcagattctagggaccactccagacctacacaatcagaaactcttgggggagggcccgaaatatctatgttttaccaagcccaccacatgattctgatgtactctaaatactgagaaaacctgttctagacaaatacccaagcaacaactccgcaggcagttaccaagtacggctggctacaactgctccatccgtgcctctttttaaagttcaaactcacaggtgactctaaggttatctacttttactcataagtaaaagccctagactggtgctaatgtcaaaccactggcctccactcaggcctccatcttctcatgccctcttaccagtatttaacttctgaggaagacaagtgatgctaaaacctgaaattccaatgaagccatatgaacagctgttcagttgcacttctaagactttacttagcagtaaattatagctcatgtgcattattttccagataacttagcttatgagtagcttatacaattatgaagatttaatattacagataaaatgtaaactgtttctttaaaattggggcttcaactttggaatttcacagcgtgctaaaataacagatttctcagaagtctttcagcaagataaacattattaagtaacttatttatgaaagtattaaaatgcttacatttgaacttgatggctaacttacaaagattctctatgtatcaaatgtaacttactgcgactaaacttaatttaatatttactctataaccaaatgaaatatatttaaaatatattgaatattttatattgttatatcctgacaagattataatattttaatgtactaatatttctgtaattatatctaaaatattattttattatattgcctaagaataaacatttgttaaattggaaaaaaaaaaaaaaaaaa

SEQ ID NO: 6 - protein, 1670 aa, Homo sapiens
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTLGYPGIPGAAGLKGQKGAPAKEEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTPGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPGPAGSDGLPGLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 7 - DNA, 10341 bp, Homo sapiens
  CDS (655)..(5727)
  sig_peptide (655)..(768)
aggctttttggttgtggcttcaagggagggaagacgtggctcaggtcccgccctcataccccctccctcccaacttaccaggcagtgtcccttctacccgggcatcgtgcccaggaggcgagaaagggctgggcgcctgaggggcaggtgggcgcggatccagggtaaggggttaaggaggtgatgctcccaaggtccgggggcccaggcggggaagtggggtgcggtcggaggtggggcgtttgggggtgtgcggtggggaggtggctgcgcggtggggaggtggctgcgcagggtggagggagtggccagcggccctgcccagattaagcgggaggcgcgccgggcgtctgcactcgggaggccccgcttgttccccgcgcccgcggcggtccgcgttccccgcctccagcccgcctccaacttgcggccgccgggcttggctgcgagagcgccgcgaagaggcagcggggcgcgggtggattggggctggaggtgcgcgtcccgtggggtggcaaggcggcagctcctggcgctgcgggcgtccccacaggaacagactttgacccagaacacagaacctcacttgtcaacaagaaccttctggaagagaagactggcagaatattttttaagtactaagacttgcctgcgatgtggtctctgcacatagtactaatgaggtgctccttcagattgaccaagtccttggccacaggtccctggtcacttatactcattctcttttctgtacaatatgtatatgggagtggaaagaaatacattggtccttgtggaggaagagattgctctgtttgccactgtgttcctgaaaaggggtctcggggtccaccaggaccaccagggccacagggtccaattggacccctgggagccccaggacccattgggctttcaggagagaaaggaatgagaggggaccgcggccctcctggagcagcaggggacaaaggagataagggtccaactggtgttcctggatttccaggtttagatggcatacctgggcacccagggcctcctggacccagaggcaaacctggtatgagtggccacaatggctcaagaggtgacccagggtttccaggaggaagaggagctcttggcccaggaggccccctaggccatcctggggaaaagggagaaaaaggaaattcagtgttcattttaggtgccgttaaaggtattcagggagacagaggggacccaggactgcctggcttaccaggatcttggggtgcaggaggaccggcaggtcccacaggatatcctggagagccagggttagtgggacctccgggccaaccagggcgtccaggtttgaagggaaatcccggtgtgggagtaaaggggcaaatgggagacccgggtgaggttggtcagcaaggttctcctggacccaccctgttggtagagccacctgacttttgtctctataaaggagaaaagggtataaaaggaattcctggaatggttggactgccaggaccaccaggacgcaagggagaatctggtattggggcaaaaggagaaaaaggtattcctggatttccagggcctcggggggatcctggttcctatggatctccaggttttccaggattaaagggagaactaggactggttggagatcctgggctatttggattaattggcccaaagggggatcctggaaatcgagggcacccaggaccaccaggtgttttggtgactccacctcttccactcaaaggcccaccaggggacccagggttccctggccgctatggagaaacaggggatgttggaccacctggtcccccaggtctcttgggcagaccaggggaagcctgtgcaggcatgataggaccccctgggccacaaggatttcctggtcttcctgggcttccaggagaagctggtattcctgggagacctgattctgctccaggaaaaccagggaagccaggatcacctggcttgcctggagcaccaggcctgcagggcctcccaggatcaagtgtgatatactgtagtgttgggaaccccggaccacaaggaataaaaggcaaagttggtcccccaggaggaagaggcccaaaaggagaaaaaggaaatgaaggactctgtgcctgtgagcctggacccatgggcccccctggccctccaggacttcctgggaggcaggggagtaagggagacttggggctccctggctggcttggaacaaaaggtgacccaggacctcctggtgctgaaggacctccagggctaccaggaaagcatggtgcctctggaccacctggcaacaaaggggcgaagggtgacatggttgtatcaagagttaaagggcacaaaggagaaagaggtcctgatgggcccccaggatttccagggcagccaggatcacatggtcgggatggacatgctggagaaaaaggggatccaggacctccaggggatcatgaagatgcgaccccaggtggtaaaggatttcctggacctctgggccccccaggcaaagcaggacctgtggggcccccaggactgggatttcctggtccaccaggagagcgaggccacccaggagttccaggccacccaggtgtgaggggccctgatggcttgaagggtcagaaaggtgacacaatttcttgcaacgtaacctaccctgggaggcatggccctccaggttttgatggacctccaggtccgaagggatttccaggtccccaaggtgcccctgggctgagtggttcagatgggcataaaggcagacctggcacaccaggaacagcggaaataccaggtccacctggttttcgtggtgacatgggagatccgggttttggaggtgaaaaggggtcctcccctgttgggcccccaggccctcccggctcaccaggagtgaatggtcagaaaggaatcccgggagaccctgcatttggtcacctgggacccccgggaaagaggggtctttcaggagtgccagggataaaaggacccagaggtgatccgggatgtccaggggctgaagggccagctggcattcctggattcctaggtctcaaaggtcccaaaggcagagagggacatgctgggtttccaggtgtcccaggtccacctggccattcctgtgaaagaggtgctccagggataccagggcaaccgggactccctgggtatccaggtagcccaggtgctccaggtgggaaaggacagccgggagatgtggggcctcccgggccagctggaatgaaaggcctccccggactcccaggacggcctggggcacatggtcccccaggcctcccaggaatcccaggtccctttggagatgatgggctacctggtcctccaggtccaaagggaccccgggggctgcctggtttcccaggttttcccggagaaagaggaaagcctggtgcagagggatgtcctggcgcaaagggagaacctggagagaagggcatgtctggccttcctggagaccggggactgagaggggccaaaggagccataggacctcccggagatgaaggagaaatggctatcatttcacaaaagggaacacctggggaacctggacctcctggagatgatggattcccaggagaaagaggtgataaaggaactcccgggatgcaagggagaagaggagagccgggaagatacggaccacctggatttcacagaggggaacctggtgagaaaggtcagccagggcctcctggacccccaggccctccaggctcaactggtctaagagggttcattggttttccaggacttccaggtgaccagggtgagccaggttctccaggtccccctggattttcaggaattgatggagcaagaggacctaaaggaaacaaaggtgaccctgccagtcactttggtccacctggtccaaagggtgagccaggtagccctggatgtccagggcattttggagcatccggagagcagggcttgcctggtattcaagggcccagaggatcacctggaaggccagggccacctggctcctctggaccaccagggtgcccaggtgatcacgggatgcctgggctgaggggacagccaggagaaatgggagaccctgggccaagaggcctccagggggatccagggataccaggtcctccgggaataaaaggtccctccggatcacctggcctgaacggcttgcatggattgaaaggtcagaaaggaactaaaggtgcttcaggtttgcatgatgtggggccacctggtccagtgggaatacctgggctaaaaggggagagaggagaccctgggagcccaggaatctctcctccaggtcctcgtggaaagaaaggtcccccaggacccccagggagttcaggaccacctggtcctgcaggtgccacaggaagagctcctaaggacattcctgacccgggtccacctggagatcagggacctcctggtcctgatggcccaagaggagcacctgggcctccaggcctccctgggagtgttgaccttctgagaggggagccaggtgactgtggtctaccagggccaccaggtccccctggcccaccaggccctccaggatacaaaggctttccaggatgtgatggaaaagatggccagaaaggaccagtgggattcccgggaccgcagggaccacatggatttcctgggccacctggagagaagggtttacctggacctccagggagaaaagggcccactggtcttccgggtcccagaggtgaaccggggccacctgcagatgtggatgactgtccccgaatcccaggccttcctggggcgccaggcatgagaggaccagaaggagccatggggctccctggaatgagaggcccctcaggaccagggtgcaaaggagagcctgggctggatggcaggaggggtgtggatggcgtccctgggtctcctgggcctcccggacgtaaaggtgacacaggagaagacggctaccctggaggaccagggcctcctggtcccattggggatcctgggcccaaagggtttggccctggatacctcggtggcttcctcctggttctccacagtcagacggaccaggagcccacctgccccctgggcatgcccaggctctggactgggtatagtctgttatacctggaagggcaagagaaagctcacaatcaagaccttggtctggcagggtcttgccttcccgtatttagcacgctgccctttgcctactgcaacatccaccaggtgtgccactatgcccagagaaacgacagatcctactggctggccagcgctgcgcccctccccatgatgccactctctgaagaggcgatccgcccctatgtcagccgctgtgcggtatgcgaggccccggcccaggcggtggcggtgcacagccaggaccagtccatccccccatgtccgcagacctggaggagcctctggatcgggtattcattcctgatgcacacaggagctggggaccaaggaggagggcaggcccttatgtcacctggcagctgcctggaagatttcagagcagcaccattccttgaatgccagggccggcagggaacttgccactttttcgcaaataagtatagcttctggctcacaacggtgaaagcagacttgcagttttcctctgctccagcaccagacaccttaaaagaaagccaggcccaacgccagaaaatcagccggtgccaggtctgcgtgaagtatagctagagaatgcgaaattcaccaacacgtggccaagagaaacttcctagggggctaagacttcctagactgtgctaagagatgtcatggtgctcattttggactccctttcagtggttccttctgttttgtcgtggttattcccagagtcctctggttccttacacattaagcaaatgctgcacagatggatttgtttggacctcccaatctagggagcctagatactcttattttactgaggatgatcgaagaactggctttacttaaaaatatgctaattctcagaaaggcaagtagatgataaaggccagattacaaattacattactgaaaacttcattcttgggttaacagtatctcaaacaattgaagtcaattactctataatacagtgggcttctggatggattttataggaaaaaataaacaggtcaatgaatgaaactagaaagcagagattttcaacatttcaaaatgatttcctctgtaatctatttttccatatactttaaataatggtaaaaccatgacgcaaagagagatttttttttaaagaagaaaaaaaaaaattcacactgccagcgttaacagttcctttcaaaggagaatgaatcatgatggcaggaaggcccacagtcgccgtattccagagatgcgacattagcataaacacatcacagatgaatataaaacattatgttctcttctgcatttttcagagaatagaaatgcctactttggcaacccttttgaaaagtagcaattatggaaaaaaaatattcaataagagattaggagcctaaaagctattagtgaatattaaggtagttattcacaaaaattgactccccattgcagtgaacttccagacagactgcttttccccagtcggggtccggcgtgtcacaggtgcgtgcgtgctaatgggactgacgctacatggggctcactcaggcaggcacgcgcttcatacaaagcatctcactcccctccccaggagagcctgcacagcttcttgcactcacaacggacactttgctccacacacataatggcagctcacacagggacgtgacagagctatcattatgcacttgggagaaaattaagggccgatttaattaaacttaggtaagaagattcatttaagtcagggttaccccatcaggaggacatggcactatctttaaacgaaacaaagacaatttataatttgaattttatgctcccgtggttggctgttacaggagcatcccattttgccaattttaaagacattcttatatttcatatcagtcttgtaccaaggcaacagtttgacatttggcattagtattttctaaaaaagtttagaatgtgtgtcaatttataatgattatttttttctgtaaagcaaaagatccctttttctgttttgctaggaatttggtgatctaatcctaaatttaaaagatttgttggaaaaaatttttaggaaactcaccttcctcatctaaaagaaaaaggcattttagagaaaactaaagaaatttctcatgagcgtgacactcattttagtgctttgtttccgtgcacttaaaaataattgagaagaaaaactcaattaaaattttgtttataagaaatgttttccttgccaaaccttgatttgtaatgagctcttatatgcagaacacatttcaaatgagttttgctatgggctgcccccagggtggcaatttattttaagagtattttctggtaaaaagaaaaatgtgtattttaagatgaaatattttcttgatgtagcagaatatttcctagttcatttgacccatttgatattttttaaaccatgctctggcatgttgaatatttttgtgcacctaaaacttaagccaatttcaatcttatttgtgattacctttctccttcccaaaaagctttatctattaccaaaagtcaaccctcctaaaagttcaacctgttcatcttgaacttggcctgagaacattttctgggaagaggtaagggtgacaaatggaacatcagaaacgtatcttgcttgctaattattttaaacactttaatgttggtattagaatattatcttcataagttaataaataagtaaaaaaaaaaaacccacacaaagacaggctaaattatgtagacagtttactccacagtgagcttgggaaggtatcagactgttcaaaatgttagtattacaagagctgtctctaaatgcagcattaacactgccctctaattttacaaaagtattttaaaggatcaaaagggcggaaaaataacttcttggaatagaagatcctgttaccttaaaccattattttcttcacctggttatatatcaatgtgccttcaattaccctttctctgtggaggtgatgaacaaagcttttaagttgttctcctaacccattattggagatgatccttatcaaacccttctctccctgcctccccaccaaccttgtagactggaacgtgcctagattgccctaatgggaactgttctgttctgtgcccgtgctggaaatctgggatgcttccttctgggttctgattctcagtgagtggagcccattcatccaggcaggtttccatgctatgagtggccctttcgttctgctctcacactccccacattgaaagctgagtcatcgctgctttggatggctacctcctctttagccccgtgcccgtctcacacagcctttgttttccacctccacccttgcagtaggcatctcccagttggtgccttagtttcaaatcccagttgccatctaagatccacccaaacttgattttgtcaccctctggattagaatccttctgtgactccgctgcccatgaagtccaaatgacttcctatggggtgaaaggtccagtggggactgttcgagggttccctctaggagtatttcctgtcatgatgaactgtgtgcagctctgcagacactcccagcacctcttcgactttgctcctgcttcaccagtgatgtcctccctggggtcagtttaccctgcgtgggaggtgaattccctgggggaaacttaggctcccttctgctgctcttgtgcagtgcacggggctgtcctcattttctccgttccccattgcttctttaatgtctcagtgcacttccttcattccattgccaaaatgcaagccccttgagggcagggaccatgcttgactcgtttttgtatgcctaatgcctagcacagagttgaacacattactgccaattcatttttttcaatgacaattccataaacatcattaggcattaggtgttcagctctgcatataaggtcttcatcagcaagacggttacaagtataaaccatttcctatttcagaatattttaatattcaacatatttgtatacccacattctcttgttacaattattgccagaaagccaattgataagctttcaaaatgggcttatcacacccctccaaaaatagatttttgttttttctataaacagaaatttcagaaatgagatttgagagatccgactgagaagacccttcagtaaaatgtgacatgacttaccttatgctgtaatctgtattccctcaggatttaagagcttcctagcataaaatctttgctaaacgtttgtgtacatgttattggtgctccgaaaaatagtcaatggaatggtgcactacaattgggctttatcatcttattttgaacctcgtgtagattacagtaaagcaaaccattcagtgtgttccaggaaattatattagatctgtgtttctatcagctcactggaataatctgataattgttacttttacttggtatggctgcaagaatagtggaaagaagaggacttgagagttggtcgaacccaggtttgaaatctggcttagtcatttttcagctgttatcctgggtgagttttgccaactttcttagcttcatttccctcattaatacggtgggacataacagctactcttgcattgaaaattaagttagattacctgtctagcatacccatcatcatacacttactatggtcacattttgtatttaaaataaactaatacgaaaaatatttctttttttttacacagaattatgattctcacagggtatataaattactgattagaattatttatatgtggccaattcttaatgtcattgggaatgctgtttcatttcaatcctcaaagttactgtagcacagaaaatatcacaatttcctgcagggacattatcagtaagttctgcagggaacaaacaattgacattaaaaatcagtactctgcaattgtcactgttatcatctgctagaaactctgcataatgcattttaaaccaccaaggctggctgccacatccatgtgaaatgcttgaattttatggtgcttaaatatttaatgattcatgggaaaaatgtgaaatgtgtctaataaattgcatccctttctctaacctctggttgtaaagttaaagactttcagcatgtaacttttgcaagatgcctggtctgccattggcacttaaatatttgttgtattacgatttataatatggttcattatatataaaattctgtgatgacttccaaaaacaa

SEQ ID NO: 8 - protein, 1690 aa, Homo sapiens
MWSLHIVLMRCSFRLTKSLATGPWSLILILFSVQYVYGSGKKYIGPCGGRDCSVCHCVPEKGSRGPPGPPGPQGPIGPLGAPGPIGLSGEKGMRGDRGPPGAAGDKGDKGPTGVPGFPGLDGIPGHPGPPGPRGKPGMSGHNGSRGDPGFPGGRGALGPGGPLGHPGEKGEKGNSVFILGAVKGIQGDRGDPGLPGLPGSWGAGGPAGPTGYPGEPGLVGPPGQPGRPGLKGNPGVGVKGQMGDPGEVGQQGSPGPTLLVEPPDFCLYKGEKGIKGIPGMVGLPGPPGRKGESGIGAKGEKGIPGFPGPRGDPGSYGSPGFPGLKGELGLVGDPGLFGLIGPKGDPGNRGHPGPPGVLVTPPLPLKGPPGDPGFPGRYGETGDVGPPGPPGLLGRPGEACAGMIGPPGPQGFPGLPGLPGEAGIPGRPDSAPGKPGKPGSPGLPGAPGLQGLPGSSVIYCSVGNPGPQGIKGKVGPPGGRGPKGEKGNEGLCACEPGPMGPPGPPGLPGRQGSKGDLGLPGWLGTKGDPGPPGAEGPPGLPGKHGASGPPGNKGAKGDMVVSRVKGHKGERGPDGPPGFPGQPGSHGRDGHAGEKGDPGPPGDHEDATPGGKGFPGPLGPPGKAGPVGPPGLGFPGPPGERGHPGVPGHPGVRGPDGLKGQKGDTISCNVTYPGRHGPPGFDGPPGPKGFPGPQGAPGLSGSDGHKGRPGTPGTAEIPGPPGFRGDMGDPGFGGEKGSSPVGPPGPPGSPGVNGQKGIPGDPAFGHLGPPGKRGLSGVPGIKGPRGDPGCPGAEGPAGIPGFLGLKGPKGREGHAGFPGVPGPPGHSCERGAPGIPGQPGLPGYPGSPGAPGGKGQPGDVGPPGPAGMKGLPGLPGRPGAHGPPGLPGIPGPFGDDGLPGPPGPKGPRGLPGFPGFPGERGKPGAEGCPGAKGEPGEKGMSGLPGDRGLRGAKGAIGPPGDEGEMAIISQKGTPGEPGPPGDDGFPGERGDKGTPGMQGRRGEPGRYGPPGFHRGEPGEKGQPGPPGPPGPPGSTGLRGFIGFPGLPGDQGEPGSPGPPGFSGIDGARGPKGNKGDPASHFGPPGPKGEPGSPGCPGHFGASGEQGLPGIQGPRGSPGRPGPPGSSGPPGCPGDHGMPGLRGQPGEMGDPGPRGLQGDPGIPGPPGIKGPSGSPGLNGLHGLKGQKGTKGASGLHDVGPPGPVGIPGLKGERGDPGSPGISPPGPRGKKGPPGPPGSSGPPGPAGATGRAPKDIPDPGPPGDQGPPGPDGPRGAPGPPGLPGSVDLLRGEPGDCGLPGPPGPPGPPGPPGYKGFPGCDGKDGQKGPVGFPGPQGPHGFPGPPGEKGLPGPPGRKGPTGLPGPRGEPGPPADVDDCPRIPGLPGAPGMRGPEGAMGLPGMRGPSGPGCKGEPGLDGRRGVDGVPGSPGPPGRKGDTGEDGYPGGPGPPGPIGDPGPKGFGPGYLGGFLLVLHSQTDQEPTCPLGMPRLWTGYSLLYLEGQEKAHNQDLGLAGSCLPVFSTLPFAYCNIHQVCHYAQRNDRSYWLASAAPLPMMPLSEEAIRPYVSRCAVCEAPAQAVAVHSQDQSIPPCPQTWRSLWIGYSFLMHTGAGDQGGGQALMSPGSCLEDFRAAPFLECQGRQGTCHFFANKYSFWLTTVKADLQFSSAPAPDTLKESQAQRQKISRCQVCVKYS

SEQ ID NO: 9 - DNA, 6531 bp, Homo sapiens
  CDS (289)..(5364)
  sig_peptide (289)..(366)
agatctgtaggaattgagtgaagaaaaagtttgcaagtctggacagaagggaaaagttctcctgagagaccggctttggggaggggaggggggaaggaagagtagctccttcttcttcttcttttttttttcttccactcttaaaaagcttctttctcttcacccaagcctcactgtccctctccggctctagctctctccatataaaccctcaagattatgtcaattggttagagccagccgggaatttcgtgcgggtgctgaaggagctgcgggagccggagaagaatgaaactgcgtggagtcagcctggctgccggcttgttcttactggccctgagtctttgggggcagcctgcagaggctgcggcttgctatgggtgttctccaggatcaaagtgtgactgcagtggcataaaaggggaaaagggagagagagggtttccaggtttggaaggacacccaggattgcctggatttccaggtccagaagggcctccggggcctcggggacaaaagggtgatgatggaattccagggccaccaggaccaaaaggaatcagaggtcctcctggacttcctggatttccagggacaccaggtcttcctggaatgccaggccacgatggggccccaggacctcaaggtattcccggatgcaatggaaccaagggagaacgtggatttccaggcagtcccggttttcctggtttacagggtcctccaggaccccctgggatcccaggtatgaagggtgaaccaggtagtataattatgtcatcactgccaggaccaaagggtaatccaggatatccaggtcctcctggaatacaaggcctacctggtcccactggtataccagggccaattggtcccccaggaccaccaggtttgatgggccctcctggtccaccaggacttccaggacctaaggggaatatgggcttaaatttccagggacccaaaggtgaaaaaggtgagcaaggtcttcagggcccacctgggccacctgggcagatcagtgaacagaaaagaccaattgatgtagagtttcagaaaggagatcagggacttcctggtgaccgagggcctcctggacctccagggatacgtggtcctccaggtcccccaggtggtgagaaaggtgagaagggtgagcaaggagagccaggcaaaagaggtaaaccaggcaaagatggagaaaatggccaaccaggaattcctggtttgcctggtgatcctggttaccctggtgaacccggaagggatggtgaaaagggccaaaaaggtgacactggcccacctggacctcctggacttgtaattcctagacctgggactggtataactataggagaaaaaggaaacattgggttgcctgggttgcctggagaaaaaggagagcgaggatttcctggaatacagggtccacctggccttcctggacctccaggggctgcagttatgggtcctcctggccctcctggatttcctggagaaaggggtcagaaaggtgatgaaggaccacctggaatttccattcctggacctcctggacttgacggacagcctggggctcctgggcttccagggcctcctggccctgctggccctcacattcctcctagtgatgagatatgtgaaccaggccctccaggccccccaggatctccaggtgataaaggactccaaggagaacaaggagtgaaaggtgacaaaggtgacacttgcttcaactgcattggaactggtatttcagggcctccaggtcaacctggtttgccaggtctcccaggtcctccaggatctcttggtttccctggacagaaaggggaaaaaggacaagctggtgcaactggtcccaaaggattaccaggcattccaggagctccaggtgctccaggctttcctggatctaaaggtgaacctggtgatatcctcacttttccaggaatgaagggtgacaaaggagagttgggttcccctggagctccagggcttcctggtttacctggcactcctggacaggatggattgccagggcttcctggcccgaaaggagagcctggtggaattacttttaagggtgaaagaggtccccctgggaacccaggtttaccaggcctcccagggaatatagggcctatgggtccccctggtttcggccctccaggcccagtaggtgaaaaaggcatacaaggtgtggcaggaaatccaggccagccaggaataccaggtcctaaaggggatccaggtcagactataacccagccggggaagcctggcttgcctggtaacccaggcagagatggtgatgtaggtcttccaggtgaccctggacttccagggcaaccaggcttgccagggatacctggtagcaaaggagaaccaggtatccctggaattgggcttcctggaccacctggtcccaaaggctttcctggaattccaggacctccaggagcacctgggacacctggaagaattggtctagaaggccctcctgggccacccggctttccaggaccaaagggtgaaccaggatttgcattacctgggccacctgggccaccaggacttccaggtttcaaaggagcacttggtccaaaaggtgatcgtggtttcccaggacctccgggtcctccaggacgcactggcttagatgggctccctggaccaaaaggtgatgttggaccaaatggacaacctggaccaatgggacctcctgggctgccaggaataggtgttcagggaccaccaggaccaccagggattcctgggccaataggtcaacctggtttacatggaataccaggagagaagggggatccaggacctcctggacttgatgttccaggacccccaggtgaaagaggcagtccagggatccccggagcacctggtcctataggacctccaggatcaccagggcttccaggaaaagcaggtgcctctggatttccaggtaccaaaggtgaaatgggtatgatgggacctccaggcccaccaggacctttgggaattcctggcaggagtggtgtacctggtcttaaaggtgatgatggcttgcagggtcagccaggacttcctggccctacaggagaaaaaggtagtaaaggagagcctggccttccaggccctcctggaccaatggatccaaatcttctgggctcaaaaggagagaagggggaacctggcttaccaggtatacctggagtttcagggccaaaaggttatcagggtttgcctggagacccagggcaacctggactgagtggacaacctggattaccaggaccaccaggtcccaaaggtaaccctggtctccctggacagccaggtcttataggacctcctggacttaaaggaaccatcggtgatatgggttttccagggcctcagggtgtggaagggcctcctggaccttctggagttcctggacaacctggctccccaggattacctggacagaaaggcgacaaaggtgatcctggtatttcaagcattggtcttccaggtcttcctggtccaaagggtgagcctggtctgcctggatacccagggaaccctggtatcaaaggttctgtgggagatcctggtttgcccggattaccaggaacccctggagcaaaaggacaaccaggccttcctggattcccaggaaccccaggccctcctggaccaaaaggtattagtggccctcctgggaaccccggccttccaggagaacctggtcctgtaggtggtggaggtcatcctgggcaaccagggcctccaggcgaaaaaggcaaacccggtcaagatggtattcctggaccagctggacagaagggtgaaccaggtcaaccaggctttggaaacccaggaccccctggacttccaggactttctggccaaaagggtgatggaggattacctgggattccaggaaatcctggccttccaggtccaaagggcgaaccaggctttcacggtttccctggtgtgcagggtcccccaggccctcctggttctccgggtccagctctggaaggacctaaaggcaaccctgggccccaaggtcctcctgggagaccaggtcctacaggttttcaaggtctaccaggtccagaaggtcctccaggtctccctggaaatggaggtattaaaggagagaagggaaatccaggccaacctgggctacctggcttgcctggtttgaaaggagatcaaggaccaccaggactccagggtaatcctggccggccgggtctcaatggaatgaaaggagatcctggtctccctggtgttccaggattcccaggcatgaaaggacccagtggagtacctggatcagctggccctgagggggaaccgggacttattggtcctccaggtcctcctggattacctggtccttcaggacagagtatcataattaaaggagatgctggtcctccaggaatccctggccagcctgggctaaagggtctaccaggaccccaaggacctcaaggcttaccaggtccaactggccctccaggagatcctggacgcaatggactccctggctttgatggtgcaggagggcgcaaaggagacccaggtctgccaggacagccaggtacccgtggtttggatggtccccctggtccagatggattgcaaggtcccccaggtccccctggaacctcctctgttgcacatggatttcttattacacgccacagccagacaacggatgcaccacaatgcccacagggaacacttcaggtctatgaaggcttttctctcctgtatgtacaaggaaataaaagagcccacggtcaagacttggggacggctggcagctgccttcgtcgctttagtaccatgcctttcatgttctgcaacatcaataatgtttgcaactttgcttcaagaaatgactattcttactggctctctaccccagagcccatgccaatgagcatgcaacccctaaagggccagagcatccagccattcattagtcgatgtgcagtatgtgaagctccagctgtggtgatcgcagttcacagtcagacgatccagattccccattgtcctcagggatgggattctctgtggattggttattccttcatgatgcatacaagtgcaggggcagaaggctcaggtcaagccctagcctcccctggttcctgcttggaagagtttcgttcagctcccttcatcgaatgtcatgggaggggtacctgtaactactatgccaactcctacagcttttggctggcaactgtagatgtgtcagacatgttcagtaaacctcagtcagaaacgctgaaagcaggagacttgaggacacgaattagccgatgtcaagtgtgcatgaagaggacataacattttgaagaattccttttgtgttttaaaatgtgatatatatatatataaaattcctaggatgcagtgtctcattgtccccaactttactactgctgccgtcaatggtgctactatatatgatcaagataacatgctgactagtaaccatgaagattcagatgtacctcagcaatgcgccagagcaaagtctctattatttttctactaaagaaataaggaagtgaatttactttttgggtccagaatgactttctccaagaattataagatgaaaattatatattttgcccagttactaaaatggtacattaaaaattcaattaagagaagagtcacattgagtaaaataaaagactgcagtttgtgggaagaattatttttcacggtgctactaatcctgctgtatcccgggtttttaatataaaggtgttaagcttattttgctttgtaagtaaagaatgtgtatattgtgaacagccttttagctcaaaatgttgagtcatttacatatgacatagcatgaatcactctttacagaaaatgtaggaaaccctagaatacagacagcaatattttatattcatgtttatcaaagtgagaggacttatattcctacatcaagttactactgagagtaaatttattttgagttttatcccgtaagttctgttttgattttttttaaaaaacaaacccttttagtcactttaatcagaattttaaatgttcatgttacataccaaattataatatctaatggagcaatttgtcttttgctatattctccaagattatctcttaagaccatatgccccctgttttaatgtttcttacatcttgtttttactcatttctgactggacaaagttcttccaaacaattctgagaaacaaaaacacacacgcagaattaacaattcttttccctgtgcttcttatgtaagaatcctcctgtggcctctgcttgtacagaactgggaaacaacacttggttagtctcttttaagttacaaaaagccaattgatgtttcttattctttttaaattttaaatattttgttataaatactcacaggataccttatttccctagctatcatctcctgacttaatgttttttaaacccaccaatataaatttaattaaagatatatgttgtaaggat

SEQ ID NO: 10 - protein, 1691 aa, Homo sapiens
MKLRGVSLAAGLFLLALSLWGQPAEAAACYGCSPGSKCDCSGIKGEKGERGFPGLEGHPGLPGFPGPEGPPGPRGQKGDDGIPGPPGPKGIRGPPGLPGFPGTPGLPGMPGHDGAPGPQGIPGCNGTKGERGFPGSPGFPGLQGPPGPPGIPGMKGEPGSIIMSSLPGPKGNPGYPGPPGIQGLPGPTGIPGPIGPPGPPGLMGPPGPPGLPGPKGNMGLNFQGPKGEKGEQGLQGPPGPPGQISEQKRPIDVEFQKGDQGLPGDRGPPGPPGIRGPPGPPGGEKGEKGEQGEPGKRGKPGKDGENGQPGIPGLPGDPGYPGEPGRDGEKGQKGDTGPPGPPGLVIPRPGTGITIGEKGNIGLPGLPGEKGERGFPGIQGPPGLPGPPGAAVMGPPGPPGFPGERGQKGDEGPPGISIPGPPGLDGQPGAPGLPGPPGPAGPHIPPSDEICEPGPPGPPGSPGDKGLQGEQGVKGDKGDTCFNCIGTGISGPPGQPGLPGLPGPPGSLGFPGQKGEKGQAGATGPKGLPGIPGAPGAPGFPGSKGEPGDILTFPGMKGDKGELGSPGAPGLPGLPGTPGQDGLPGLPGPKGEPGGITFKGERGPPGNPGLPGLPGNIGPMGPPGFGPPGPVGEKGIQGVAGNPGQPGIPGPKGDPGQTITQPGKPGLPGNPGRDGDVGLPGDPGLPGQPGLPGIPGSKGEPGIPGIGLPGPPGPKGFPGIPGPPGAPGTPGRIGLEGPPGPPGFPGPKGEPGFALPGPPGPPGLPGFKGALGPKGDRGFPGPPGPPGRTGLDGLPGPKGDVGPNGQPGPMGPPGLPGIGVQGPPGPPGIPGPIGQPGLHGIPGEKGDPGPPGLDVPGPPGERGSPGIPGAPGPIGPPGSPGLPGKAGASGFPGTKGEMGMMGPPGPPGPLGIPGRSGVPGLKGDDGLQGQPGLPGPTGEKGSKGEPGLPGPPGPMDPNLLGSKGEKGEPGLPGIPGVSGPKGYQGLPGDPGQPGLSGQPGLPGPPGPKGNPGLPGQPGLIGPPGLKGTIGDMGFPGPQGVEGPPGPSGVPGQPGSPGLPGQKGDKGDPGISSIGLPGLPGPKGEPGLPGYPGNPGIKGSVGDPGLPGLPGTPGAKGQPGLPGFPGTPGPPGPKGISGPPGNPGLPGEPGPVGGGGHPGQPGPPGEKGKPGQDGIPGPAGQKGEPGQPGFGNPGPPGLPGLSGQKGDGGLPGIPGNPGLPGPKGEPGFHGFPGVQGPPGPPGSPGPALEGPKGNPGPQGPPGRPGPTGFQGLPGPEGPPGLPGNGGIKGEKGNPGQPGLPGLPGLKGDQGPPGLQGNPGRPGLNGMKGDPGLPGVPGFPGMKGPSGVPGSAGPEGEPGLIGPPGPPGLPGPSGQSIIIKGDAGPPGIPGQPGLKGLPGPQGPQGLPGPTGPPGDPGRNGLPGFDGAGGRKGDPGLPGQPGTRGLDGPPGPDGLQGPPGPPGTSSVAHGFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 11 - DNA, 6774 bp, Homo sapiens
  CDS (235)..(5358)
  sig_peptide (235)..(297)
atggaactatctcctgagtgctgcaagttgtaacgggcaccgctgagcctgtttccctttggagcacttcttatctagaagcagtgtttagtttcttccaaactgggccacttcgtccacctactctgttctgagtaaggaaacagcctccaagcatcagcagagcccagatgagcacgggccgcggagccgcttagcagtctcccgggacccagctccggaggagccgcaagcatgcaccctgggttgtggctgctcctggttacgttgtgcctgaccgaggaactggcagcagcgggagagaagtcttatggaaagccatgtgggggccaggactgcagtgggagctgtcagtgttttcctgagaaaggagcgagaggacgacctggaccaattggaattcaaggcccaacaggtcctcaaggattcactggctctactggtttatcgggattgaaaggagaaaggggtttcccaggccttctgggaccttatggaccaaaaggagataagggtcccatgggagttcctggctttcttggcatcaatgggattccgggccaccctggacaaccaggccccagaggcccacctggtctggatggctgtaatggaactcaaggagctgttggatttccaggccctgatggctatcctgggcttctcggaccacccgggcttcctggtcagaaaggatcaaaaggtgaccctgtccttgctccaggtagtttcaaaggaatgaagggggatcctgggctgcctggactggatggaatcactggcccacaaggagcacccggatttcctggagctgtaggacctgcaggaccaccaggattacaaggtcctccagggcctcctggtcctcttggtcctgatgggaatatggggctaggttttcaaggagagaaaggagtcaagggggatgttggcctccctggcccagcaggacctccaccatctactggagagctggaattcatgggattccccaaagggaagaaaggatccaagggtgaaccagggcctaagggttttccaggcataagtggccctccaggcttcccgggccttggaactactggagaaaagggagaaaagggagaaaagggaatccctggtttgccaggacctaggggtcccatgggttcagaaggagtccaaggccctccagggcaacagggcaagaaagggaccctgggatttcctgggcttaatggattccaaggaattgagggtcaaaagggtgacattggcctgccaggcccagatgttttcatcgatatagatggtgctgtgatctcaggtaatcctggagatcctggtgtacctggcctcccaggccttaaaggagatgaaggcatccaaggcctacgtggcccttctggtgtccctggattgccagcattatcaggtgtcccaggagccctagggcctcagggatttccagggctgaagggggaccaaggaaacccaggccgtaccacaattggagcagctggcctccctggcagagatggtttgccaggcccaccaggtccaccaggcccacctagtccagaatttgagactgaaactctacacaacaaagagtcagggttccctggtctccgaggagaacaaggtccaaaaggaaacctaggcctcaaaggaataaaaggagactcaggtttctgtgcttgtgacggtggtgttcccaacactggaccacccggggaaccaggcccacctggtccatggggtctcataggccttccaggccttaaaggagccagaggagatcgaggctctgggggtgcacagggcccagcaggggctccaggcttagttgggcctctgggtccttcaggacccaaaggaaagaagggggaaccaattctcagtacaatccaaggaatgccaggagatcggggtgattctggctcccagggcttccgtggtgtaataggagaaccaggcaaggacggagtaccaggtttaccaggtctgccaggccttccgggtgatggtggacagggcttcccaggtgaaaaggggttacctggacttcctggtgaaaaaggccatcctggtccacctggcctcccaggaaatgggttaccaggacttcctggaccccgtgggcttcctggagataaaggcaaggatggattaccgggacaacaaggccttcccggatctaagggtgactgctgctgtagagaggttggtaaaggagacttagacacagagagaggaatcaccctgccctgtattattcctgggtcatacggtccatcaggatttccaggcactcccggattcccaggccctaaagggtctcgaggcctccctgggaccccaggccagcctgggtcaagtggaagtaaaggagagccagggagtccaggattggttcatcttcctgaattaccaggatttcctggacctcgtggggagaagggcttgcctgggtttcctgggctccctggaaaagatggcttgcctgggatgattggcagtccaggcttacctggttccaagggagccactggtgacatctttggtgctgaaaatggtgctccgggggaacaaggcctacaaggattaacagggcacaaaggatttcttggagactctggccttccaggactcaagggtgtgcacgggaagcctggcttactaggccccaaaggtgagcggggcagccctgggacaccaggacaggtgggacagccaggcaccccaggatctagtggtccatatggcatcaagggcaaatctgggctcccaggagcaccaggcttcccaggcatctcaggacatcctggaaagaaaggaacaagaggcaagaaaggtcctcctggatcaattgtaaagaaagggctgccagggctaaaaggccttcctggaaatccaggcctagtaggactgaaaggaagcccaggctctccaggggtcgctgggttgccagccctctctggacccaagggagagaaggggtctgttggattcgtaggttttccaggaataccaggtctgcctggtattcctggaacaagaggattaaaaggaattccaggatcaactggaaaaatgggaccatctggacgtgctggtactcctggtgaaaagggagacagaggcaatccggggccagtcggaatacctagtccaagacgtccaatgtcaaacctttggctcaaaggagacaaaggctctcaaggctcagccggatccaatggatttcctgggccaagaggtgacaaaggagaggctggtcgacctggaccaccaggcctacctggagctcctggcctcccaggcattatcaaaggagttagtggaaagccagggccccctggcttcatgggaatccggggcttacctggcctgaaggggtcctctgggatcacaggtttcccaggaatgccaggagaaagtggttcacaaggtatcagagggtcgcctggactcccaggagcatctggtctcccaggcctgaaaggagacaacggccagacagttgaaatttccggtagcccaggacccaagggacagcctggcgaatctggttttaaaggcacaaaaggaagagatggactaataggcaatataggcttccctggaaacaaaggtgaagatggaaaagttggtgtttctggagatgttggccttcctggagctccaggatttccaggagttgccggcatgagaggagaaccaggacttccaggttcttctggtcaccaaggggcaattgggcctctaggatcccccggattaataggacccaaaggcttccctggatttcctggtttacatggactgaatgggcttccgggcaccaagggtacccatggcactccaggacctagtatcaccggtgtgcctgggcctgctggtctccctggacccaaaggagaaaaaggatatccaggaattggcatcggagctccagggaagccgggcctgagagggcaaaaaggtgatcgaggtttcccaggtctccagggccctgctggtctccccggtgccccaggcatctccttgccctcactcatagcaggacagcctggtgaccccgggcgaccaggcctagatggagaacgaggccgcccaggccccgctggacccccaggtccccctgggccatcctcgaatcaaggcgacaccggagaccctggcttccctggaattcctggacctaaagggcctaagggagaccaaggaattccaggtttttctggcctccctggagagctaggactgaaaggcatgagaggtgagcctggcttcatggggactccaggcaaggttgggccacctggagacccaggatttcccggaatgaaggggaaggcagggccaagaggctcttctggcctccaaggtgatcctggacaaacaccaactgcagaagctgtccaggttcctcctggacccttgggtctaccagggatcgatggcatccctggcctcactggggaccctggggctcaaggccctgtaggcctacaaggctccaaaggtttacctggcatccccggtaaagatggccccagtgggctcccaggcccacctggggctcttggtgatcctggtctgcctggactgcaaggccctccaggatttgaaggagctccagggcagcaaggccccttcgggatgcctggaatgcctggccagagcatgagagtgggctacacgttggtaaagcacagccagtcggaacaggtgcccccgtgtcccatcgggatgagccagctgtgggtggggtacagcttactgtttgtggaggggcaagagaaagcccacaaccaggacctgggctttgctggctcctgtctgccccgcttcagcaccatgcccttcatctactgcaacatcaacgaggtgtgccactatgccaggcgcaatgataaatcttactggctctccactaccgcccctatccccatgatgcccgtcagccagacccagattccccagtacatcagccgctgctctgtgtgtgaggcaccctcgcaagccattgctgtgcacagccaggacatcaccatcccgcagtgccccctgggctggcgcagcctctggattgggtactctttcctcatgcacactgccgctggtgccgagggtggaggccagtccctggtctcacctggctcctgcctagaggactttcgggccactcctttcatcgaatgcagtggtgcccgaggcacctgccactactttgcaaacaagtacagtttctggttgaccacagtggaggagaggcagcagtttggggagttgcctgtgtctgaaacgctgaaagctgggcagctccacactcgagtcagtcgctgccaggtgtgtatgaaaagcctgtagggtggcacctgccactctgccccttgccctcccctgcccctcacaacagtcacctcacaaacctgaatggtctgaagaaggaaggcctgagcccctttgcctgtcaagttgtacattggagtctcatttgggctagactaccggacactcgtcaccccagccctcgggtccatagagatgagcccaccctgctgagatctgctgtcctgtttctgtcaagctggtgctactgtttgatttggatgattgtgtgactattcatggctacctcagaaagatttgatgggccacaactgtcttagactgctagctttctccttaccgtcttgatcggaaagctcttcctaatcgctaatcagtcatttcttcatgtacagaggtcagcacacattatttggcttaaaccagaacccagtgtttccacacttaaattctctaaccgaatattcatggatggctcaagtctgcacagagcaagtcctcactcttcaaggaggcccactgtgtctaggcaggcaagagaattgaaatgaggtgccacccagtagcccagagtgagctttagctctctctagaatgagcaagactgggccccacatggcttagagaggcttgaaggccagcagctgggttgggggtggtggtcattaatggcatatggtcctagacaaaccatctcctccttgccggctccccctccagccagagacagaggatgtggcctggttcaaagtaaagcagaggatgcaacaaatgtggccaagcctatcaaaggaaatgagaatgacagccttttttcctgggccagaagtagaggggtgggtgcgtaaggatgtgtgagttttgcttttgactccaggaacaaaaaggtaaatcccacatcccagtttctcagaagtccctgtttattccaaatgccatccagatgtgtgcaatgtggcaaactgaagctgcacagtgttggtttccttgtattctgaggatgttaaagactttgttaaatggttatccaattgctctttcacaggtagcctattaaactattttaatatgtttttttaaacctcataaaaatctagcacactcttctcttgagcagttagcagacctaaagcaagcctgaattggctatgcagtacattgtattctgtttgggggaatttgttttagccattttctttaattaccagttttccagaacactcttagctatgttgacatgaggcagttccttccaggtgattctgtttccttaagtattatataaactgtgccaatacagacaaagcataatcaatataatctgaattattgttatctttacctcctgagtaataagcatggtgtcagttttgtacatagcaaataaaataaatgaaatctgaacatgtgaaaaaaaaaaaaaaaaaaaaaaaa

SEQ ID NO: 12 - protein, 1707 aa, Homo sapiens
MHPGLWLLLVTLCLTEELAAAGEKSYGKPCGGQDCSGSCQCFPEKGARGRPGPIGIQGPTGPQGFTGSTGLSGLKGERGFPGLLGPYGPKGDKGPMGVPGFLGINGIPGHPGQPGPRGPPGLDGCNGTQGAVGFPGPDGYPGLLGPPGLPGQKGSKGDPVLAPGSFKGMKGDPGLPGLDGITGPQGAPGFPGAVGPAGPPGLQGPPGPPGPLGPDGNMGLGFQGEKGVKGDVGLPGPAGPPPSTGELEFMGFPKGKKGSKGEPGPKGFPGISGPPGFPGLGTTGEKGEKGEKGIPGLPGPRGPMGSEGVQGPPGQQGKKGTLGFPGLNGFQGIEGQKGDIGLPGPDVFIDIDGAVISGNPGDPGVPGLPGLKGDEGIQGLRGPSGVPGLPALSGVPGALGPQGFPGLKGDQGNPGRTTIGAAGLPGRDGLPGPPGPPGPPSPEFETETLHNKESGFPGLRGEQGPKGNLGLKGIKGDSGFCACDGGVPNTGPPGEPGPPGPWGLIGLPGLKGARGDRGSGGAQGPAGAPGLVGPLGPSGPKGKKGEPILSTIQGMPGDRGDSGSQGFRGVIGEPGKDGVPGLPGLPGLPGDGGQGFPGEKGLPGLPGEKGHPGPPGLPGNGLPGLPGPRGLPGDKGKDGLPGQQGLPGSKGDCCCREVGKGDLDTERGITLPCIIPGSYGPSGFPGTPGFPGPKGSRGLPGTPGQPGSSGSKGEPGSPGLVHLPELPGFPGPRGEKGLPGFPGLPGKDGLPGMIGSPGLPGSKGATGDIFGAENGAPGEQGLQGLTGHKGFLGDSGLPGLKGVHGKPGLLGPKGERGSPGTPGQVGQPGTPGSSGPYGIKGKSGLPGAPGFPGISGHPGKKGTRGKKGPPGSIVKKGLPGLKGLPGNPGLVGLKGSPGSPGVAGLPALSGPKGEKGSVGFVGFPGIPGLPGIPGTRGLKGIPGSTGKMGPSGRAGTPGEKGDRGNPGPVGIPSPRRPMSNLWLKGDKGSQGSAGSNGFPGPRGDKGEAGRPGPPGLPGAPGLPGIIKGVSGKPGPPGFMGIRGLPGLKGSSGITGFPGMPGESGSQGIRGSPGLPGASGLPGLKGDNGQTVEISGSPGPKGQPGESGFKGTKGRDGLIGNIGFPGNKGEDGKVGVSGDVGLPGAPGFPGVAGMRGEPGLPGSSGHQGAIGPLGSPGLIGPKGFPGFPGLHGLNGLPGTKGTHGTPGPSITGVPGPAGLPGPKGEKGYPGIGIGAPGKPGLRGQKGDRGFPGLQGPAGLPGAPGISLPSLIAGQPGDPGRPGLDGERGRPGPAGPPGPPGPSSNQGDTGDPGFPGIPGPKGPKGDQGIPGFSGLPGELGLKGMRGEPGFMGTPGKVGPPGDPGFPGMKGKAGPRGSSGLQGDPGQTPTAEAVQVPPGPLGLPGIDGIPGLTGDPGAQGPVGLQGSKGLPGIPGKDGPSGLPGPPGALGDPGLPGLQGPPGFEGAPGQQGPFGMPGMPGQSMRVGYTLVKHSQSEQVPPCPIGMSQLWVGYSLLFVEGQEKAHNQDLGFAGSCLPRFSTMPFIYCNINEVCHYARRNDKSYWLSTTAPIPMMPVSQTQIPQYISRCSVCEAPSQAIAVHSQDITIPQCPLGWRSLWIGYSFLMHTAAGAEGGGQSLVSPGSCLEDFRATPFIECSGARGTCHYFANKYSFWLTTVEERQQFGELPVSETLKAGQLHTRVSRCQVCMKSL

SEQ ID NO: 13 - DNA, 36 bp, Artificial Sequence
  primer
ccctcctggttctccggatccagctctggaaggacc

SEQ ID NO: 14 - DNA, 36 bp, Artificial Sequence
  primer
ggtccttccagagctggatccggagaaccaggaggg

SEQ ID NO: 15 - DNA, 38 bp, Artificial Sequence
  primer
gtcccccaggccctcctgtttctccgggtccagctctg

SEQ ID NO: 16 - DNA, 38 bp, Artificial Sequence
  primer
cagagctggacccggagaaacaggagggcctgggggac

SEQ ID NO: 17 - RNA, 19 nt, Artificial Sequence
  PPIA siRNA
caagaugacuaaugucaaa

SEQ ID NO: 18 - RNA, 19 nt, Artificial Sequence
  PPIA siRNA
uuugacauuagucaucuug

SEQ ID NO: 19 - RNA, 25 nt, Artificial Sequence
  PPIB siRNA
ggucucuucggaaagacuguuccaa

SEQ ID NO: 20 - RNA, 25 nt, Artificial Sequence
  PPIB siRNA
uuggaacagucuuuccgaagagacc

SEQ ID NO: 21 - RNA, 25 nt, Artificial Sequence
  PPIC siRNA
gcaacaggagagaaaggauauggau

SEQ ID NO: 22 - RNA, 25 nt, Artificial Sequence
  PPIC siRNA
auccauauccuuucucuccuguugc

SEQ ID NO: 23 - RNA, 25 nt, Artificial Sequence
  PPID siRNA
gccaaguaauuaaaggaauaggagu

SEQ ID NO: 24 - RNA, 25 nt, Artificial Sequence
  PPID siRNA
acuccuauuccuuuaauuacuuggc

SEQ ID NO: 25 - RNA, 25 nt, Artificial Sequence
  PPIE siRNA
ccaaugagaauuaaggaaggcucuu

SEQ ID NO: 26 - RNA, 25 nt, Artificial Sequence
  PPIE siRNA
aagagccuuccuuaauucucauugg

SEQ ID NO: 27 - RNA, 19 nt, Artificial Sequence
  PPIF siRNA
ggcagaugucgucccaaag

SEQ ID NO: 28 - RNA, 19 nt, Artificial Sequence
  PPIF siRNA
cuuugggacgacaucugcc

SEQ ID NO: 29 - RNA, 25 nt, Artificial Sequence
  PPIG siRNA
gggauaagagugaguugaaugaaau

SEQ ID NO: 30 - RNA, 25 nt, Artificial Sequence
  PPIG siRNA
auuucauucaacucacucuuauccc

SEQ ID NO: 31 - RNA, 25 nt, Artificial Sequence
  PPIH siRNA
cgugguguucuuugaugucaguauu

SEQ ID NO: 32 - RNA, 25 nt, Artificial Sequence
  PPIH siRNA
aauacugacaucaaagaacaccacg

SEQ ID NO: 33 - RNA, 25 nt, Artificial Sequence
  PPIL1 siRNA
gaacuuugcugaguuggcucgucga

SEQ ID NO: 34 - RNA, 25 nt, Artificial Sequence
  PPIL1 siRNA
ucgacgagccaacucagcaaaguuc

SEQ ID NO: 35 - RNA, 25 nt, Artificial Sequence
  NKTR siRNA
ggaaagcagcauguccgaaaguaaa

SEQ ID NO: 36 - RNA, 25 nt, Artificial Sequence
  NKTR siRNA
uuuacuuucggacaugcugcuuucc

SEQ ID NO: 37 - RNA, 25 nt, Artificial Sequence
  PPWD1 siRNA
gacccaacaauagucuguacaucau

SEQ ID NO: 38 - RNA, 25 nt, Artificial Sequence
  PPWD1 siRNA
augauguacagacuauuguuggguc

SEQ ID NO: 39 - DNA, 34 bp, Artificial Sequence
  loxP
ataacttcgtatagcatacattatacgaagttat

SEQ ID NO: 40 - DNA, 34 bp, Artificial Sequence
  loxKMR3
ataacttcgtatagcatacattataccttgttat

SEQ ID NO: 41 - DNA, 34 bp, Artificial Sequence
  lox2272
ataacttcgtataggatactttatacgaagttat

SEQ ID NO: 42 - DNA, 30 bp, Artificial Sequence
  primer
aagggttccggatccccatcaagcttatcg

SEQ ID NO: 43 - DNA, 28 bp, Artificial Sequence
  primer
gggtagggatggaagttcaccaaagccc

SEQ ID NO: 44 - DNA, 36 bp, Artificial Sequence
  primer
cctgtgctcactctttagtagatcaattttgcatcc

SEQ ID NO: 45 - DNA, 30 bp, Artificial Sequence
  primer
gctacacataatgacatacacctgaggctg

SEQ ID NO: 46 - DNA, 32 bp, Artificial Sequence
  primer
ccgagctcgtcgacaagctcgaattagcttgg

SEQ ID NO: 47 - DNA, 31 bp, Artificial Sequence
  primer
gagtcaatttgaccatgctgtgctgctgaag

SEQ ID NO: 48 - DNA, 32 bp, Artificial Sequence
  primer
ggaggtgacatgtgccagtctgcgggaactgg

SEQ ID NO: 49 - DNA, 31 bp, Artificial Sequence; identical to SEQ ID NO: 47
  primer

SEQ ID NO: 50 - DNA, 36 bp, Artificial Sequence
  primer
acacagatctgatatcatcgatgaattcgagcttgc

SEQ ID NO: 51 - DNA, 27 bp, Artificial Sequence
  primer
ggtcggtgggagagactggtcttcctg

SEQ ID NO: 52 - DNA, 25 bp, Artificial Sequence
  primer
tggagcctggtactccaatgggtcc

SEQ ID NO: 53 - protein, 20 aa, Artificial Sequence
  inhibitor
EFGGVMCVESVNREMSPLVD

SEQ ID NO: 54 - protein, 20 aa, Artificial Sequence
  inhibitor
REMSPLVDNIALWMTEYLNR

SEQ ID NO: 55 - protein, 20 aa, Artificial Sequence
  inhibitor
MCVESVNREMSPLVDNIALW

SEQ ID NO: 56 - protein, 20 aa, Artificial Sequence
  inhibitor
LLSLALVGACITLGAYLGHK

SEQ ID NO: 57 - DNA, 20 bp, Artificial Sequence
  siRNA
gtcctcccactcttagagcc

SEQ ID NO: 58 - DNA, 20 bp, Artificial Sequence
  siRNA
gtcctcccactcttagagcc

SEQ ID NO: 59 - DNA, 20 bp, Artificial Sequence
  siRNA
cttcccgcctgtgccattgt

SEQ ID NO: 60 - DNA, 20 bp, Artificial Sequence
  siRNA
gatgtcctcccactcttaga

SEQ ID NO: 61 - DNA, 21 bp, Artificial Sequence
  siRNA
tgtcctcccactcttagagcc

SEQ ID NO: 62 - RNA, 24 nt, Artificial Sequence
  siRNA
ggaggacauccaagaagauuguca

SEQ ID NO: 63 - RNA, 27 nt, Artificial Sequence
  siRNA
augacaaucuucuuggauguccuccca

SEQ ID NO: 64 - RNA, 25 nt, Artificial Sequence
  siRNA
cccaaagacagcugagaacuucaga

SEQ ID NO: 65 - RNA, 27 nt, Artificial Sequence
  siRNA
ucugaaguucucagcugucuuugggac

SEQ ID NO: 66 - RNA, 25 nt, Artificial Sequence
  siRNA
gcuccaccuuccacagggugauccc

SEQ ID NO: 67 - RNA, 27 nt, Artificial Sequence
  siRNA
gggaucacccuguggaagguggagccu

SEQ ID NO: 68 - RNA, 24 nt, Artificial Sequence
  siRNA
cagacugguuggauggcaagcaug

SEQ ID NO: 69 - RNA, 27 nt, Artificial Sequence
  siRNA
acaugcuugccauccaaccagucuguc

SEQ ID NO: 70 - RNA, 25 nt, Artificial Sequence
  siRNA
ggcuaaugcugguccuaacaccaac

SEQ ID NO: 71 - RNA, 27 nt, Artificial Sequence
  siRNA
guugguguuaggaccagcauuagccau

(19) United States
(12) Patent Application Publication
(10) Pub. No.: US 2023/0082311 A1
(43) Pub. Date: Mar. 16, 2023

(54) DRUG FOR CURATIVE THERAPY OF INTRACTABLE HEREDITARY RENAL ALPORT SYNDROME

(71) Applicant: NATIONAL UNIVERSITY CORPORATION KUMAMOTO UNIVERSITY, Kumamoto (JP)

(72) Inventors: Hirofumi Kai, Kumamoto (JP); Tsuyoshi Shuto, Kumamoato (JP); Mary Ann SUICO, Kumamoto (JP); Sachiko Tsukamoto, Kumamoto (JP); Shogo Misumi, Kumamoto (JP); Teppei Kawahara, Kumamoto (JP); Yuki Hitora, Kumamoto (JP); Hikaru Kato, Kumamoto (JP); Shunsuke Kotani, Kumamoto (JP); Kimi Araki, Kumamoto (JP)

(21) Appl. No.: 17/642,612

(22) PCT Filed: Sep. 11, 2020
(86) PCT No.: PCT/JP2020/034538

§ 371 Date: Mar. 11, 2022

(30) Foreign Application Priority Data
Sep. 11, 2019 (JP) 2019-165583

(51) Int. Cl. A61K 38/13; A61P 13/12; C07K 16/40; C12N 15/113; C12N 15/115

(52) U.S. Cl. CPC A61K 38/13; A61P 13/12; C07K 16/40; C12N 15/1137; C12N 15/115; C12N 2310/11; C12N 2310/141; C12N 2310/531; C12N 2310/14; C12N 2310/16

(57) ABSTRACT

by screening using a high-throughput evaluation system relating to the promotion of the extracellular secretion of trimers of the causative proteins COL4A3/A4/A5, which decreased in the renal tissue of patients with Alport syndrome (AS), it was found that cyclosporin A has an effect of promoting extracellular secretion of trimers of type-IV collagen. From further studies, it was found that Alisporivir and NIM258, which do not inhibit calcineurin, also have an action to promote extracellular secretion of type-IV collagen. Furthermore, the present inventors have found that these actions are based on the cyclophilin D inhibitory mechanism, and have found a radical therapeutic agent for AS by inhibition of cyclophilin D. The present invention provides a composition and an AS therapeutic/prophylactic drug, which contain a cyclophilin D inhibitor as an active ingredient, for promoting the secretion of collagen trimers in cells having a mutated type-IV collagen gene.

### CROSS REFERENCES TO RELATED APPLICATIONS

This application is a U.S. national stage of International Application No. PCT/JP2020/034538, filed Sep. 11, 2020, which claims priority based on Japanese Patent Application No. 2019-165583 filed with the Japan Patent Office on Sep. 11, 2019, and the entire contents of International Application No. PCT/JP2020/034538 and Japanese Patent Application No. 2019-165583 are incorporated in this application by reference.

### INCORPORATION BY REFERENCE OF MATERIAL SUBMITTED ELECTRONICALLY

A Sequence Listing, which is a part of the present disclosure, is submitted concurrently with the specification as a text file. The name of the text file containing the Sequence Listing is “57701_Seqlisting.txt.” The Sequence Listing was created on Mar. 11, 2022, and is 270,434 Bytes in size. The subject matter of the Sequence Listing is incorporated by reference herein in its entirety.

### TECHNICAL FIELD

The present invention relates to a collagen trimer secretion promoter, particularly to the field of treatment of Alport syndrome.

### BACKGROUND ART

Alport syndrome (AS) is a hereditary disease caused by a mutation in any of the type-IV collagen genes (COL4A3, COL4A4, COL4A5) that constitute the basement membrane of the glomerulus of the kidney. It is developed from childhood in many cases and is a serious disease that reaches the end-stage renal disease in the teens and twenties. Currently, the treatment of AS is symptomatic therapy with a rennin-angiotensin (RAS) inhibitor, as in other chronic kidney diseases. Patients eventually progress to end-stage renal failure without exception, and are forced to undergo dialysis or kidney transplantation.

Cyclosporin A (CsA) is a hydrophobic cyclic polypeptide consisting of 11 amino acids. CsA is used as an immunosuppressant for suppressing rejection during transplantation, and the like because it suppresses the production of cytokines by inhibiting calcineurin. In 1992, Callis et al. reported that administration of CsA delays the onset of AS, and suggested the possibility of treating AS with CsA (Non Patent Literature 1). Thereafter, an attempt was made to elucidate the mechanism, and the action on the hemodynamics of the glomerulus, and the like were also reported (Non Patent Literatures 2 and 3). In 2008, Faul et al. reported that in podocytes, CsA maintains phosphorylation of synaptopodin by inhibiting calcineurin and protects synaptopodin—which is an actin-interacting protein—from degradation by cathepsin L (Non Patent Literature 4). Based on this report, the mechanism of action by CsA has been considered to be the stabilization of the actin skeleton in podocytes (Non Patent Literatures 5 and 6).

On the other hand, however, it has been reported that CsA also has side effects such as nephrotoxicity associated with inhibition of the calcineurin-NFAT pathway (Non Patent Literature 7).

Alisporivir is a compound reported as a cyclosporine derivative (Patent Literature 1), and has been developed as a therapeutic agent for hepatitis C virus.

As widely used AS models, COL4A3 deficient (Col4a3 KO) mouse (Non Patent Literature 8) and COL4A5 nonsense mutant (Col4a5-G5X) mouse (Non Patent Literature 9) can be mentioned. Both of them are models in which the expression itself of causative proteins α3 (IV) and α5 (IV) has been lost. Col4a5 G5X mutant mouse was produced by Dr. Michelle Rheault et al. of University of Minnesota, based on the mutant type of a boy who shows renal pathology from the age of 6. This mouse has a mutation at the fifth codon of Exon1 converted from Glycine to a stop codon, and shows almost no expression of α5(IV) variant since the mutation probably accompanies a nonsense mutation-dependent mRNA degradation mechanism, etc., and α345(IV) on the renal glomerular basement membrane (GBM) is lost (Non Patent Literature 9). This mouse is a model that exhibits from continuous proteinuria leakage to inflammatory cell infiltration, focal segmental glomerulosclerosis (FSGS), interstitial fibrosis, etc., and reflects well the progressive clinical pathology of AS. Using this model mouse, the present inventors have also explored for pathological control factors and evaluated the efficacy of therapeutic drugs, and identified several therapeutic targets and therapeutic drug candidates. However, all of them target factors involved in the progression of pathological conditions and the effect of improving the renal pathology was limited. Therefore, a need for a model animal that more accurately reflects the pathology of α345(IV), which causes AS, has been recognized.

### CITATION LIST

### Patent Literature

### [PTL 1]

WO 2000/001715

### Non Patent Literatures

### [NPL 1]

L. Callis et al., Pediatric Nephrology (1992) 6: 140-144

### [NPL 2]

L. Callis et al., Kidney International (1999) 55: 1051-1056

### [NPL 3]

Dilys Chen et al., J. Am. Soc. Nephrol (2003) 14: 690-698

### [NPL 4]

Christian Faul et al., Nat. Med. (2008) 14(9): 931-938

### [NPL 5]

Massela, L et al., Pediatric Nephrology (2010) 25(7): 1269-1275

### [NPL 6]

Sugimoto, K et al., Clin. Exp. Nephrol. (2014) 18: 492-498

### [NPL 7]

Charbit, M et al., Pediatric Nephrology (2007) 22: 57-63

### [NPL 8]

Miner J H et al., J. Cell Biol. (1996) 135: 1403-1413.

### [NPL 9]

Rheault M N et al., J. Am. Soc. Nephrol. (2004) 15: 1466-1474.

### SUMMARY OF INVENTION

### Technical Problem

The present invention aims to provide a method for treating AS, that is safer and directly acts on the onset mechanism of the disease.

### Solution to Problem

The present inventors took note of a decrease in the formation and extracellular secretion of a trimer of the causative proteins COL4A3/A4/A5 that previously decreased in AS renal tissues, studied the possibility of a radical treatment method for AS by improving them, and successfully constructed a system (evaluation system by split NanoLuc) that can evaluate them with high throughput (Omachi K. et al., Cell Chem. Biol. (2018) 25: 634). Using the constructed system, they screened an independently collected microbial extract library, and clarified that CsA having the following structure promotes extracellular secretion of the trimer of type-IV collagen, which is the causative protein of AS.

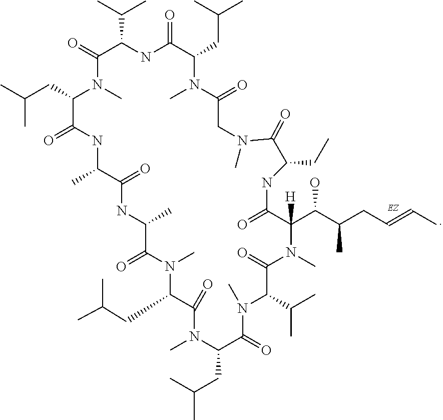 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

Until now, it has been considered that the efficacy of CsA on AS is achieved by the calcineurin inhibitory activity of CsA. Thus, PSC-833—which is a derivative of CsA but does not inhibit calcineurin and has no immunosuppressive action—was tested, and no effect of promoting the secretion of mutant collagen trimer was confirmed. Therefore, even if CsA is effective for AS, it could not be inferred that a derivative thereof has the same effect.

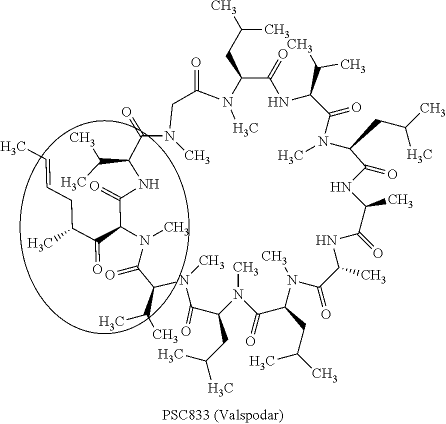 [SMILES: C/C=C/C[C@@H](C)C(=O)[C@H]1C(=O)N[C@@H](C(C)C)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

The present inventors conducted further studies of the action of CsA derivatives on AS, and unexpectedly found that Alisporivir—which does not inhibit calcineurin and has no immunosuppressive action like PSC833—has an action to promote the secretion of AS mutant collagen trimer.

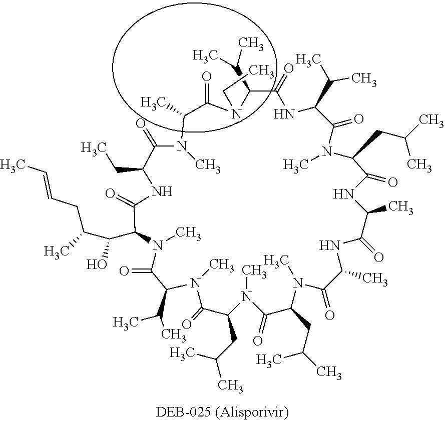

Furthermore, the present inventors have also investigated other compounds, and found that NIM258—which does not inhibit calcineurin and has no immunosuppressive action—also has an action to promote the secretion of AS mutant collagen trimer.

Therefrom the present inventors have found that CsA, Alisporivir, and NIM258 afford the extracellular secretion of type-IV collagen trimer based on a new mechanism completely different from the calcineurin pathway, and enable the radical treatment of AS, particularly that activities other than the calcineurin pathway that Alisporivir and NIM258 have achieve an AS therapeutic effect by promoting secretion of type-IV collagen trimer.

Until now, it has been considered difficult to clinically apply CsA as a therapeutic drug for AS because of the nephrotoxicity associated with its calcineurin inhibitory activity. That Alisporivir, which has already been confirmed to have no nephrotoxicity in clinical trials (S. Zeuzem et al., Aliment Pharmacol. Ther. (2015) 42: 829-844; M. Buti et al., J. of Viral Hepatitis (2015) 22: 596-606), and NIM258, which similarly has no immunosuppressive action, show a secretagogue action on type-IV collagen and are effective in treating AS means that the problem of nephrotoxicity that has prevented the clinical application of CsA can be solved, whereby the present inventors have succeeded for the first time in providing a radical therapeutic drug for AS with low nephrotoxicity.

Furthermore, the present inventors elucidated the mechanism by which CsA and Alisporivir promote the secretion of trimers of type-IV collagen, and found that this mechanism is due to the involvement of cyclophilin D. Specifically, in cells lacking cyclophilin D, the promoting action of CsA and Alisporivir on the secretion of trimers of type-IV collagen is not sufficiently exhibited. Therefore, they have found that CsA and Alisporivir promote the secretion of trimers of type-IV collagen by acting on cyclophilin D. In addition, the present inventors investigated the role of cyclophilin D in the secretion of trimers of type-IV collagen, and found that knockdown of cyclophilin D promotes the secretion of trimers of type-IV collagen.

From the above, the present inventors have found that Alisporivir promotes extracellular secretion of trimers of type-IV collagen, which is abnormal in AS, and becomes a curative drug for AS, that the mechanism of promoting the secretion of trimers of type-IV collagen by CsA and Alisporivir is based on the inhibition of cyclophilin D, and that cyclophilin D is a new target for the secretion of trimers of type-IV collagen.

On the other hand, the present inventors also analyzed the mutations of type-IV collagen for which promotion of the secretion of trimers by inhibition of cyclophilin D can be effective. As a result, it was found that the inhibition is particularly effective for deficiency in the secretion of trimers of type-IV collagen due to amino acid mutations in or around the region of Exon 41 in COL4A5.

### Advantageous Effects of Invention

The present invention promotes the extracellular secretion of trimers of type-IV collagen. Therefore, it can contribute to the radical treatment and prevention of diseases caused by deficiency in the secretion of trimers of type-IV collagen represented by AS. Particularly, the present invention is effective for diseases caused by deficiency in the secretion of trimers of type-IV collagen due to mutations in or around the region of Exon 41 in COL4A5.

### BRIEF DESCRIPTION OF DRAWINGS

FIG. 1 is a graph showing the secretion level of collagen trimer containing COL4A5 (G1244D) when treated with CsA. In the graph, the numerical value on the horizontal axis shows the concentration (μM) of CsA, and the vertical axis shows the secretion level of collagen trimer as a ratio (% of control) to the control (dimethyl sulfoxide (DMSO)-treated group). The “DMSO” on the horizontal axis shows the DMSO treatment group. The secretion level of collagen trimer increases depending on the concentration of CsA.

FIG. 2 is a graph showing the secretion level of collagen trimer containing COL4A5 (G1244D) when treated with PSC-833. In the graph, the numerical value on the horizontal axis shows the concentration (μM) of PSC-833, and the vertical axis shows the secretion level of collagen trimer as a ratio (% of control) to the control (dimethyl sulfoxide (DMSO)-treated group). The “DMSO” on the horizontal axis shows the DMSO treatment group, and “CsA” shows cyclosporine A-treated group (1 μM) (positive control). The secretion level of collagen trimer does not increase even when the amount of PSC-833 is increased to 10 μM.

FIG. 3 is a graph showing the secretion level of collagen trimer containing COL4A5 (G1244D) when treated with Alisporivir. In the graph, the numerical value on the horizontal axis shows the concentration (μM) of Alisporivir, and the vertical axis shows the secretion level of collagen trimer as a ratio (% of control) to the control (DMSO-treated group). The “DMSO” on the horizontal axis shows the DMSO treatment group, and “CsA” shows CsA-treated group (positive control). The secretion level of collagen trimer increases depending on the concentration of Alisporivir.

FIG. 4 is a graph showing the results of the experiments of FIG. 1 and FIG. 3 performed under treatment conditions with higher concentrations of CsA (5, 10 μM) and Alisporivir (5, 10 μM). In the graph, the horizontal axis shows the concentrations of cyclosporine A (CsA) and Alisporivir (ALV), and the vertical axis shows the secretion level of collagen trimer as a ratio (% of control) to the control (DMSO-treated group). The “DMSO” on the horizontal axis shows the DMSO treatment group, “ALV” shows the Alisporivir-treated group, and “CsA” shows the CsA-treated group (positive control). At higher concentrations, the secretion level of collagen trimer tended to be high for Alisporivir as compared with CsA.

FIG. 5 includes graphs showing the secretion level of collagen trimer when wild-type (α3WT/α4WT/α5WT: upper graph) and a cell line with G1241V amino acid mutation in COL4A5 (α3 and α4 are wild-type) (α3WT/α4WT/α5G1241V: lower graph) were each treated with Alisporivir. In the graph, the horizontal axis shows the concentration (μM) of Alisporivir, and the vertical axis shows the secretion level of collagen trimer as a ratio (% of control) to the control (DMSO-treated group). The “DMSO” on the horizontal axis shows the DMSO treatment group, “ALV” shows the Alisporivir-treated group, and “CsA” shows the CsA-treated group (positive control). It was shown that the trimer secretion promoting effect by CsA and Alisporivir is higher in the mutant type than in the wild type.

FIG. 6 is a graph showing the secretion level of collagen trimer when a cell line with G1241V amino acid mutation in COL4A5 (α3 and α4 are wild-type) (a3WT/a4WT/a5G1241V: lower graph) was treated with NIM258. In the graph, the horizontal axis shows the concentration (μM) of NIM258, and the vertical axis shows the secretion level of collagen trimer as a ratio (% of control) to the control (DMSO-treated group). The “DMSO” on the horizontal axis shows the DMSO treatment group, and “NIM258” shows the NIM258-treated group. A trimer secretion promoting effect by NIM258 was shown.

FIG. 7 is a graph showing the measurement results of the trimer secretion promoting ability of CsA and Alisporivir in cells of a wide range of clinically reported AS variants (G227S, G594D, G624D, G869R, S916G, G1030S, G1107R, G1140V, G1220D, G1241C, G1241V, G1244D, P1517T, and R1569Q of COL4A5), and Exon41 variants (1202nd-1263rd amino acids of COL4a5)-deleted (dExon41) variant). The vertical axis shows the secretion level of collagen trimer as a ratio (% of control) of each cell to the control (DMSO-treated group). α5WT shows wild-type COL4a5. The “CON” on the horizontal axis shows the control (DMSO-treated group), “CsA” shows the CsA-treated group (positive control), and “ALV” shows the Alisporivir-treated group. It was shown that the trimer secretion promoting effect by CsA and Alisporivir was particularly high in G1030S, G1107R, G1220D, G1241C, G1241V, G1244D, and Exon41-deleted cells.

FIG. 8 is a graph showing the examination results of the collagen trimer secretion promoting effect by adding CsA and Alisporivir to G1244D mutation-transfected HEK293T cells in which cyclophilins (PPIA (cyclophilin A), PPIB, PPIC, PPID, PPIE, PPIF (cyclophilin D), PPIG, PPIH, PPIL1, NKTR, PPWD1) were knocked down with siRNA, in order to elucidate the collagen trimer secretion promoting mechanism by CsA and Alisporivir. The vertical axis shows the secretion level of collagen trimer as a ratio (% of control) of each cell to the control (DMSO-treated group). In the horizontal axis, “CON” shows the control (DMSO-treated group), “CsA” shows the CsA-treated group (positive control), and “ALV” shows the Alisporivir-treated group. A trimer secretion promoting ability by CsA and Alisporivir was particularly inhibited in PPIF (cyclophilin D) cells.

FIG. 9 is a graph showing the results of investigating collagen trimer secreting ability in cells in which various cyclophilins (PPIA (cyclophilin A), PPIB, PPIC, PPID, PPIE, PPIF (cyclophilin D), PPIG, PPIH, PPIL1, NKTR, PPWD1) were knocked down with siRNA, using G1244D mutant-transfected HEK293T cells. The vertical axis shows the secretion level of collagen trimer as a ratio (% of control) to the control (siGL2-treated group). The horizontal axis shows the type of siRNA used. Particularly, promotion of collagen trimer secretion was confirmed in the cells treated with siPPIF.

FIG. 10 is a schematic diagram showing the nucleic acids used in producing Col4a5G1244D mouse and Col4a5ΔExon41 mouse.

FIG. 11 Photographs showing expression of Laminin in ΔExon41 mouse, and Western blot (WB) photograph (inside of frame) of cell/basement membrane lysate.

FIG. 12 is a graph showing the urinary protein level of ΔExon41 mouse. The vertical axis shows urinary protein level (mg/mg Cre), and the horizontal axis shows age in weeks (week-old). Black circles show wild-type mouse, and black squares show ΔExon41 mouse.

FIG. 13 is a graph showing the serum creatinine value of ΔExon41 mouse. The vertical axis shows serum creatinine value (mg/dL). Con in the horizontal axis shows wild-type mouse, and ΔExon41 shows ΔExon41 mouse.

FIG. 14 is a graph showing changes in the urinary protein after administration of CsA to ΔExon41 mouse. The vertical axis shows urinary protein level (mg/mg Cre), and the horizontal axis shows age in weeks (week-old). Administration of CsA suppressed an increase in the urinary protein.

### DESCRIPTION OF EMBODIMENTS

In one embodiment, the present invention relates to a composition for promoting the secretion of collagen trimers in cells having a type-IV collagen gene having a mutation, which composition contains a cyclophilin D inhibitor as an active ingredient, or an agent for promoting the secretion of collagen trimers.

As the cyclophilin D inhibitor, Alisporivir, as well as, for example, the compounds described in WO2009/018179; WO2012/103520; U.S. Pat. No. 9,132,138B; WO2014/093632; JP2016-124821A; JP2017-513490A; WO2015/200725; U.S. Pat. No. 9,738,615B; WO2016/112321; U.S. Pat. No. 10,179,161B; WO2019/173382 can be mentioned. The cyclophilin D inhibitor may also be a substance described as a cyclophilin D selective inhibitor in these documents.

Among these substances, a substance that does not show a calcineurin inhibitory action is preferred as the cyclophilin D inhibitor. Whether or not the candidate substance exhibits a calcineurin inhibitory action can be determined, for example, using a system in which a reporter gene is inserted downstream of the NFAT binding site because calcineurin is transferred into the nucleus by dephosphorylating the activated T cell nuclear factor (NF-AT) (Blood (2000) 96(2): 459-466.). When the expression of the reporter gene does not decrease in the presence of a candidate molecule, the candidate molecule can be determined to show no calcineurin inhibitory action. In the present specification, showing no calcineurin inhibitory action means that a calcineurin inhibitory action may be completely absent but generally, the degree of calcineurin inhibition is sufficiently low and does not cause problems such as toxicity when used for the purpose of the present invention. As one example, the absence of calcineurin inhibitory action may mean that the calcineurin inhibitory action is lower than that of CsA. In addition, since calcineurin inhibition appears as immunosuppressive activity as a biological activity, the cyclophilin D inhibitor may be a substance that does not exhibit immunosuppressive activity.

For example, a cyclosporine derivative represented by the following formula, or a salt thereof, or a hydrate or solvate thereof can be used as the cyclophilin D inhibitor:

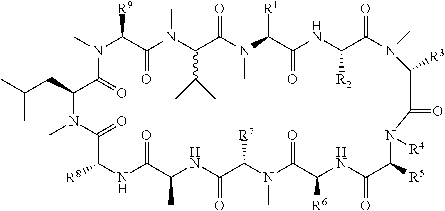

wherein
R¹ is a C1-10 alkyl group or C2-10 alkenyl group substituted by a hydroxyl group; or a group represented by the following formula,

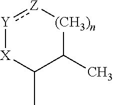

wherein

X is O or NR¹¹
R¹¹ is H or a C1-6 alkyl group,

Y is CR¹²R¹³, CR¹⁴ or >C═O,
R¹² is H, a C1-6 alkyl group, or an OH group,
R¹³ is H, a C1-6 alkyl group, or an OH group,
R¹⁴ is H or a C1-6 alkyl group,

Z is (CH₂)ₘ, CR¹⁵, NR¹⁶, or O,
R¹⁵ is H or a C1-6 alkyl group,
R¹⁶ is H or a C1-6 alkyl group,
m is an integer of 1-3,

n is 0 or 1, and

a bond between Y and Z shown by a solid line and a broken line is a single bond or a double bond, when the bond between Y and Z is a single bond, Y is CR¹²R¹³ or C═O, and Z is (CH₂)ₘ, NR¹⁶, or O; when the bond between Y and Z is a double bond, Y is CR¹⁴, and Z is CR¹⁵,

R² is a C1-4 alkyl group optionally substituted by a hydroxyl group,
R³ is a hydrogen atom; a C1-6 alkyl group, a C1-6 alkoxy group, or a C1-6 alkylthio group, each optionally substituted by a hydroxyl group and/or a halogen atom; or a group selected from the following formulas:

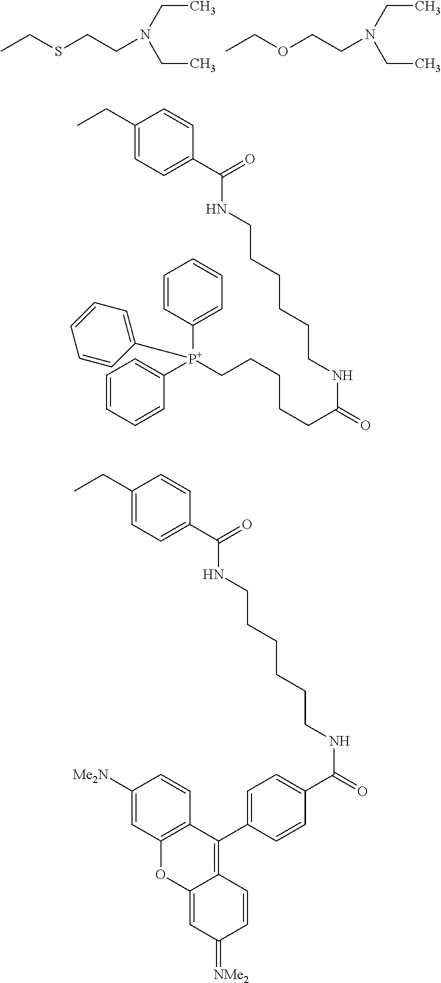

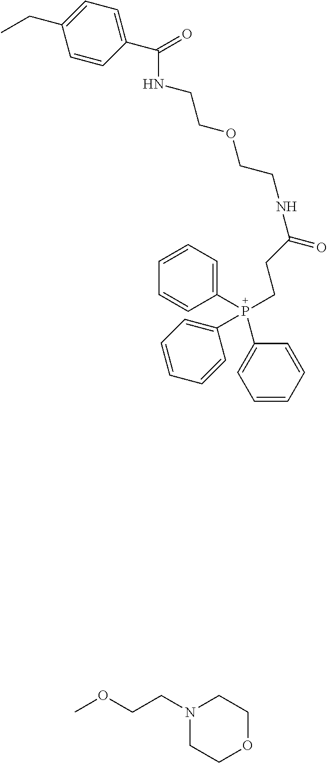

R⁴ is a C1-4 alkyl group,
R⁵ is a C1-6 alkyl group optionally substituted by a hydroxyl group and/or a C1-6 alkoxy group, or a group represented by the following:

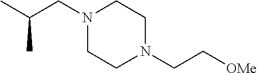

R⁶ is an n-propyl group or an isopropyl group,
R⁷ is a C1-4 alkyl group,
R⁸ is a C1-4 alkyl group optionally substituted by a hydroxyl group,
R⁹ is a branched C3-4 alkyl group, and
a bond shown by a wavy line means that a steric structure of a chiral carbon atom to be bonded is an R form, an S form, or a mixture thereof.

In the above, the “C1-10 alkyl group” means a straight chain or branched saturated hydrocarbon group having 1-10 carbon atoms, the “C1-6 alkyl group” means a straight chain or branched saturated hydrocarbon group having 1-6 carbon atoms, and the “C1-4 alkyl group” means a straight chain or branched saturated hydrocarbon group having 1-4 carbon atoms. As the alkyl group, for example, methyl group, ethyl group, n-propyl group, i-propyl group, n-butyl group, sec-butyl group, tert-butyl group, isobutyl group, pentyl group, isopentyl group, 2,3-dimethylpropyl group, hexyl group, 2-methylpentyl group, heptyl group, 2-methylhexyl group, octyl group, 2-methylheptyl group, nonyl group, 2-methyloctyl group, decyl group, and 2-methylnonyl group can be mentioned. The “branched C3-4 alkyl group” means isopropyl group, isobutyl group, sec-butyl group, or tert-butyl group.

The “C2-10 alkenyl group” means a monovalent group having 2-10 carbon atoms which is obtained by removing one hydrogen atom from any carbon atom of a straight chain or branched unsaturated hydrocarbon having one or more carbon-carbon double bonds. As the C2-10 alkenyl group, for example, vinyl group, propenyl group, isopropenyl group, 1-butenyl group, 2-butenyl group, 3-butenyl group, 1-methyl-1-propenyl group, 2-methyl-1-propenyl group, 1-methyl-2-propenyl group, 2-methyl-2-propenyl group, 1-methylidene-1-propane group, 1-pentenyl group, 1-pentenyl group, 3-pentenyl group, 4-pentenyl group, 1-methyl-1-butenyl group, 1-methyl-2-butenyl group, 1-methyl-3-butenyl group, 1-methylidenebutyl group, 2-methyl-1-butenyl group, 2-methyl-2-butenyl group, 2-methyl-3-butenyl group, 2-methylidenebutyl group, 3-methyl-1-butenyl group, 3-methyl-2-butenyl group, 3-methyl-3-butenyl group, 1-ethyl-1-propenyl group, 1-ethyl-2-propenyl group, 1-hexenyl group, 2-hexenyl group, 3-hexenyl group, 4-hexenyl group, 5-hexenyl group, 1-methyl-1-pentenyl group, 1-methyl-2-pentenyl group, 1-methyl-3-pentenyl group, 1-methyl-4-pentenyl group, 1-methylidenepentyl group, 2-methyl-1-pentenyl group, 2-methyl-2-pentenyl group, 2-methyl-3-pentenyl group, 2-methyl-4-pentenyl group, 2-methylidenepentyl group, 3-methyl-1-pentenyl group, 3-methyl-2-pentenyl group, 3-methyl-3-pentenyl group, 3-methyl-4-pentenyl group, 3-methylidenepentyl group, 4-methyl-1-pentenyl group, 4-methyl-2-pentenyl group, 4-methyl-3-pentenyl group, 4-methyl-4-pentenyl group, 1-ethyl-1-butenyl group, 1-ethyl-2-butenyl group, 1-ethyl-3-butenyl group, 2-ethyl-1-butenyl group, 2-ethyl-2-butenyl group, 2-ethyl-3-butenyl group, 1-(1-methylethyl)-1-propenyl group, 1-(1-methylethyl)-2-propenyl group, 1-ethyl-2-methyl-1-propenyl group, 1-ethyl-2-methyl-2-propenyl group, 4-hexenyl group, 2-methyl-4-pentenyl group, 4-heptenyl group, 2-methyl-4-hexenyl group, 4-octenyl group, 2-methyl-4-heptenyl group, 4-nonenyl group, 2-methyl-4-octenyl group, 4-decenyl group, and 2-methyl-4-nonenyl group can be mentioned.

The “C1-6 alkoxy group” is a group bonded to the aforementioned C1-6 alkyl group via an oxygen atom ((C1-6 alkyl group)-O-group), and the alkyl group moiety may be linear or branched. The C1-6 alkoxy group means that the number of carbon atoms in the alkyl group moiety is 1 to 6. As the alkoxy group, for example, methoxy group, ethoxy group, 1-propyloxy group, 2-propyloxy group, 2-methyl-1-propyloxy group, 2-methyl-2-propyloxy group, 2,2-dimethyl-1-propyloxy group, 1-butyloxy group, 2-butyloxy group, 2-methyl-1-butyloxy group, 3-methyl-1-butyloxy group, 2-methyl-2-butyloxy group, 3-methyl-2-butyloxy group, 1-pentyloxy group, 2-pentyloxy group, 3-pentyloxy group, 2-methyl-1-pentyloxy group, 3-methyl-1-pentyloxy group, 2-methyl-2-pentyloxy group, 3-methyl-2-pentyloxy group, 1-hexyloxy group, 2-hexyloxy group, and 3-hexyloxy group can be mentioned. The C1-6 alkoxy group is preferably C1-5 alkoxy group, more preferably methoxy group, ethoxy group, n-propyloxy group, i-propyloxy group, n-butyloxy group, sec-butyloxy group, t-butyloxy group, isobutyloxy group, pentyloxy group, isopentyloxy group, or 2,3-dimethylpropyloxy group, further preferably C1-3 alkoxy group (methoxy group, ethoxy group, and propyloxy group), further more preferably methoxy group or ethoxy group.

The “C1-6 alkylthio group” refers to a group bonded to the aforementioned C1-6 alkyl group via a sulfur atom ((C1-6 alkyl group)-S-group), and the alkyl group moiety may be linear or branched. The C1-6 alkylthio group means that the number of carbon atoms in the alkyl group moiety is 1 to 6. As the alkylthio group, for example, methylthio group, ethylthio group, 1-propylthio group, 2-propylthio group, 2-methyl-1-propylthio group, 2-methyl-2-propylthio group, 2,2-dimethyl-1-propylthio group, 1-butylthio group, 2-butylthio group, 2-methyl-1-butylthio group, 3-methyl-1-butylthio group, 2-methyl-2-butylthio group, 3-methyl-2-butylthio group, 1-pentylthio group, 2-pentylthio group, 3-pentylthio group, 2-methyl-1-pentylthio group, 3-methyl-1-pentylthio group, 2-methyl-2-pentylthio group, 3-methyl-2-pentylthio group, 1-hexylthio group, 2-hexylthio group, and 3-hexylthio group can be mentioned. The C1-6 alkylthio group is preferably C1-5 alkylthio group, more preferably methylthio group, ethylthio group, n-propylthio group, i-propylthio group, n-butylthio group, sec-butylthio group, t-butylthio group, isobutylthio group, pentylthio group, isopentylthio group, or 2,3-dimethylpropylthio group, further preferably C1-3 alkylthio group (methylthio group, ethylthio group, and propylthio group), further more preferably methylthio group or ethylthio group.

R¹ in the aforementioned formula is preferably 1-hydroxy-2-methyl-hexyl group, 1-hydroxy-2-methyl-4-hexenyl group, or a group represented by the following formula:

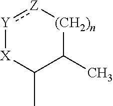

wherein

### X is O or NR¹¹

R¹¹ is H or a C1-6 alkyl group,

Y is CR¹²R¹³, CR¹⁴ or >C═O,

R¹² is H, a C1-6 alkyl group, or an OH group,

R¹³ is H, a C1-6 alkyl group, or an OH group,

R¹⁴ is H or a C1-6 alkyl group,

Z is (CH₂)ₘ, CR¹⁵, NR¹⁶, or O,

R¹⁵ is H or a C1-6 alkyl group,

R¹⁶ is H or a C1-6 alkyl group,

m is an integer of 1-3,

n is 0 or 1, and
a bond between Y and Z shown by a solid line and a broken line is a single bond or a double bond, when the bond between Y and Z is a single bond, Y is CR¹²R¹³ or C═O, and Z is (CH₂)ₘ, NR¹⁶, or O; when the bond between Y and Z is a double bond, Y is CR¹⁴, and Z is CR¹⁵.

R¹ may be, for example, a 1-hydroxy-2-methyl-hexyl group or a 1-hydroxy-2-methyl-4-hexenyl group.

R² is preferably a C1-3 alkyl group optionally substituted by a hydroxyl group, and may be, for example, a methyl group, an ethyl group, a 1-hydroxyethyl group, an isopropyl group, or an n-propyl group.

R³ is preferably a hydrogen atom or a C1-4 alkyl group optionally substituted by a hydroxyl group and/or a halogen atom, and may be, for example, a hydrogen atom or a methyl group.

R⁴ is preferably a methyl group.

R⁵ is preferably a C3-4 branched alkyl group, for example, an isobutyl group or a sec-butyl group.

R⁶ is preferably a C3 alkyl group, for example, an isopropyl group.

R⁷ is preferably a C4 alkyl group, for example, an isobutyl group.

R⁸ is preferably a methyl group or a hydroxymethyl group, more preferably a methyl group.

R⁹ is preferably a C3-4 branched alkyl group, for example, an isobutyl group or an isopropyl group.

More specifically, as the cyclophilin D inhibitor, cyclosporine A, cyclosporine D, NIM258, NIM811, Alisporivir, PKF220-384, SCY-635, mtCsA1, mtCsA2, mtCsA3, cyclosporine C, cyclosporine G, cyclosporine M, cyclosporine H, dihydrocyclosporine D, [(D)Ser]8-cyclosporine, [MeIle]8-cyclosporine, [MeAla]6-cyclosporine, [(D)Pro]3-cyclosporine, and SCY-641 can be mentioned. Among these, a substance that does not show a calcineurin inhibitory/immunity suppressive activity is preferred. It is specifically NIM258 (J. Fu et al., J Med Chem (2014) 57: 8503?16.), NIM811 (R. Traber et al., Antiviral Chemistry&Chemotherapy (1994) 5: 331-9), Alisporivir (J. Paeshuyse et al., Hepatology (2006) 43: 761-70.), PKF220-384 (Molecular pharmacology 62(1): 22-9), SCY-635 (S. Hopkins et al., Antimicrob Agents Chemother (2010) 54: 660-72.), mtCsA1, mtCsA2, mtCsA3 (the above from Biochem J. 2012; 441(Pt3): 901-7.), cyclosporine H (J Immunol 1991; 147: 1940-1946), or [MeIle]8-cyclosporine (SciFinder), more preferably, derivative compound that shares the same skeleton as Alisporivir (NIM258, NIM811, SCY-635) (Jiping Fu et al., J. Med. Chem. (2014) 57: 8503-8516), most preferably Alisporivir or NIM258.

In one embodiment, the present invention may be a composition for promoting secretion of collagen trimers in cells having a type-IV collagen gene having a mutation, wherein the composition contains a cyclophilin D inhibitor as an active ingredient.

As the cyclophilin D inhibitor, siRNA, shRNA, or miRNA against cyclophilin D, an anti-cyclophilin antibody, an aptamer against cyclophilin D, or an antisense nucleic acid molecule against cyclophilin D can also be used. The cyclophilin D inhibitor may be a peptide having the following sequence:

(SEQ ID NO: 53)
EFGGVMCVESVNREMSPLVD
(SEQ ID NO: 54)
REMSPLVDNIALWMTEYLNR
(SEQ ID NO: 55)
MCVESVNREMSPLVDNIALW
(SEQ ID NO: 56)
LLSLALVGACITLGAYLGHK

As the nucleic acid molecule such as siRNA and antisense, for example, nucleic acid molecules having the following sequences can be mentioned (left is 5′-terminus, right is 3′-terminus):

(SEQ ID NO: 27)
GGCAGAUGUCGUCCCAAAG
(SEQ ID NO: 28)
CUUUGGGACGACAUCUGCC
(SEQ ID NO: 57)
GTCCTCCCACTCTTAGAGCC
(SEQ ID NO: 58)
GTCCTCCCACTCTTAGAGCC
(SEQ ID NO: 59)
CTTCCCGCCTGTGCCATTGT
(SEQ ID NO: 60)
GATGTCCTCCCACTCTTAGA
(SEQ ID NO: 61)
TGTCCTCCCACTCTTAGAGCC
(SEQ ID NO: 62)
GGAGGACAUCCAAGAAGAUUGUCAT
(SEQ ID NO: 63)
AUGACAAUCUUCUUGGAUGUCCUCCCA
(SEQ ID NO: 64)
CCCAAAGACAGCUGAGAACUUCAGA
(SEQ ID NO: 65)
UCUGAAGUUCUCAGCUGUCUUUGGGAC
(SEQ ID NO: 66)
GCUCCACCUUCCACAGGGUGAUCCC
(SEQ ID NO: 67)
GGGAUCACCCUGUGGAAGGUGGAGCCU
(SEQ ID NO: 68)
CAGACUGGUUGGAUGGCAAGCAUG
(SEQ ID NO: 69)
ACAUGCUUGCCAUCCAACCAGUCUGUC
(SEQ ID NO: 70)
GGCUAAUGCUGGUCCUAACACCAAC
(SEQ ID NO: 71)
GUUGGUGUUAGGACCAGCAUUAGCCAU

These cyclophilin D inhibitors can be obtained, for example, by the methods described in WO2009/018179; WO2012/103520; U.S. Pat. No. 9,132,138B; WO2014/093632; JP2016-124821A; JP2017-513490A; WO2015/200725; U.S. Pat. No. 9,738,615B; WO2016/112321; U.S. Pat. No. 10,179,161B; WO2019/173382.

The cyclophilin D inhibitor is a compound represented by the aforementioned formula, or the like. In the present specification, the cyclophilin D inhibitor may be in the form of, where necessary, a pharmacologically acceptable salt and/or hydrate or solvate thereof. In the present specification, the “pharmacologically acceptable salt” is a salt formed by combining the compound of the present invention with an inorganic or organic acid, and is acceptable for administration to the body as a medicament. Such salt is described in, for example, Berge et al., J. Pharm. Sci. 66: 1-19 (1977), and the like. As the salt, salts with mineral acids such as hydrochloric acid, hydrobromic acid, sulfuric acid, nitric acid, phosphoric acid, and the like; salts with organic acids such as methanesulfonic acid, benzenesulfonic acid, p-toluenesulfonic acid, acetic acid, propionic acid, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid, salicylic acid, and the like; and salts with acidic amino acids such as aspartic acid, glutamic acid, and the like; can be mentioned. A hydrate or solvate of a cyclophilin D inhibitor, and a hydrate or solvate of a pharmacologically acceptable salt of a cyclophilin D inhibitor may also be encompassed in the cyclophilin D inhibitor. In the present specification, unless it is clearly unsuitable, the “cyclophilin D inhibitor” also includes, even if it is not explicitly indicated, a pharmacologically acceptable salt, a hydrate and a solvate of a cyclophilin D inhibitor, as well as a hydrate or solvate of a pharmacologically acceptable salt of a cyclophilin D inhibitor.

Type-IV collagen is a major constituent component of the basement membrane, and includes 6 types (α1, 2, 3, 4, 5, and 6) (SEQ ID NO: 2, 4, 6, 8, 10, and 12, respectively), each of which is encoded by the corresponding gene (COL4A1, 2, 3, 4, 5, and 6) (SEQ ID NO: 1, 3, 5, 7, 9, and 11, respectively). In collagen, three a chains are associated to form a triple helix structure. In the glomerular basement membrane (GBM) of the kidney, a3, a4, and a5 are mainly expressed, and mutations in these are considered to be often involved particularly in the onset of AS. In the present specification, the “collagen” optionally means a type-IV collagen. In the present specification, moreover, the “type-IV collagen” optionally means a type-IV collagen having a mutation. In the present specification, the type-IV collagen gene having a mutation is preferably a type-IV collagen gene (COL4A3, COL4A4, or COL4A5), most preferably COL4A5.

In the present specification, the “mutation” means, unless inconsistent to understand as such, a mutation due to substitution, deletion, and/or insertion of an amino acid, preferably substitution. In one embodiment, the “mutation” of collagen is a mutation that reduces the amount of collagen trimer secreted, or a mutation that results in AS.

The mutation in the type-IV collagen gene is not particularly limited as long as it is a mutation that contributes to the secretion of collagen trimers, and is preferably a mutation that reduces the secretion of collagen trimers. For example, mutation selected from G129E, G227S, G230C, G325R, G426R, G475S, G521D, G573D, G594D, G594S, G624D, G650D, L664N, G675S, G796R, G869R, G911E, S916G, G953V, G1030S, G1107R, G1140V, G1143D, G1220D, G1241C, G1241V, G1244D, G1448R, P1517T, C1567R, R1569Q, M1607I, L1649R, and R1683Q in COL4A5 (preferably, G1241V and/or G1244D), or amino acid mutation in Exon41 can be mentioned. In the present specification, AS having such mutation is preferred.

“Promoting the secretion of collagen trimer” means that the extracellular secretion level of collagen consequently increases as compared with the case of non-administration of the drug. It may include any or two or three kinds of actions from promotion of collagen trimer secretion itself, increase of collagen gene production, and promotion of collagen trimer secretion. The composition of the present invention enables a radical treatment of AS caused by insufficient secretion levels of collagen trimer by promoting the secretion of collagen trimer.

The composition of the present invention can be a therapeutic or prophylactic agent for a disease or condition, represented by AS, which is based on the deficiency in the secretion of collagen trimers. In the present specification, AS may be read as appropriate as a disease or condition based on the deficiency in the secretion of collagen trimers. AS is typically a hereditary disease, and representative clinical symptoms thereof include renal lesions such as glomerulonephritis, chronic nephritis, hematuria (particularly, persistent hematuria), proteinuria, wide range of irregular thickening of the glomerular basement membrane, and changes in the reticulation of dense layer of the glomerular basement membrane; ear lesions such as deafness, particularly, sensorineural (neurotic) deafness, hearing loss and progressive exacerbation in high frequency region; eye lesions such as anterior lenticonus, posterior subcapsular cataract, posterior polymorphous dystrophy, and blind spot retina; complications of diffuse leiomyomatosis; and the like.

The patients to be treated/prevented with the therapeutic or prophylactic drug of the present invention may have an amino acid mutation G1030S, G1107R of COL4A5, or in the region of Exon41, where the mutation in the region of Exon41 may be G1220D, G1241C, G1241V, or G1244D.

Therefore, in another embodiment, the present invention relates to the above-mentioned composition (medical composition) which is a therapeutic drug, a prophylactic drug, or an improving agent for AS. The medical composition of the present invention can be formulated as a medical composition for oral or parenteral administration, for example, tablet, powder, granule, capsule, internal liquid, syrup, ointment, lotion, or injection (e.g., injection for intravenous injection, injection for subcutaneous administration, injection for muscle injection, drip). In the present specification, the medical composition can be prepared according to a conventional method.

The medical composition may contain a pharmacologically acceptable carrier (additive for preparation). The type of preparation additive used in the production of the medical composition, the ratio of the preparation additive to the active ingredient, or the method for producing the medical composition can be appropriately selected by those skilled in the art according to the form of the composition. As the preparation additive, an inorganic or organic substance, or a solid or liquid substance can be used, and generally, it can be added in a proportion of 1 wt % to 90 wt % based on the weight of the active ingredient.

The medical composition may be further administered with other medicaments. The medical composition of the present invention and other medicament may be contained in a single preparation or may be administered together as separate preparations. As such other medicament, a medicament that does not inhibit the therapeutic effect of a cyclophilin D inhibitor on AS, but is known to have a therapeutic effect on AS, or a medicament that enhances the therapeutic effect of a cyclophilin D inhibitor can be mentioned. As the medicament that enhances the therapeutic effect of a cyclophilin D inhibitor, rennin-angiotensin-aldosterone system inhibitors: angiotensin converting enzyme inhibitors (e.g., enalapril, ramipril, benazepril, and the like), angiotensin II receptor antagonists (e.g., valsartan, losartan, candesartan, and the like); statin (e.g., fluvastatin and the like); aldosteron antagonists (e.g., spironolactone and the like); activated vitamin D2 analogues (e.g., paricalcitol and the like); and the like can be mentioned.

The present invention relates to a method for treating, preventing, or improving AS, including administering an effective amount of a cyclophilin D inhibitor to a patient in need thereof. The present invention relates to a method for improving deficiency in secreting collagen trimers in a patient, or an agent for promoting secretion of collagen trimers in a patient, each including administering an effective amount of a cyclophilin D inhibitor to a patient in need thereof. The “patient in need thereof” is specifically a patient affected with AS. The patient is preferably a patient having an amino acid mutation G1030S, G1107R, or in the region of Exon41 in COL4A5, where the mutation in the region of Exon41 may be G1220D, G1241C, G1241V, or G1244D. For example, the therapeutic or prophylactic method of the present invention may include a step of examining whether the patient to be the subject of treatment or prophylaxis has such a mutation. Specifically, the present invention may be directed to a method for the treatment or prophylaxis of AS, including examining a gene mutation in COL4A5 of a patient, and when the aforementioned gene mutation is an amino acid mutation G1030S, G1107R, or in the region of Exon41, administering an effective amount of the composition of the present invention to the patient. In this method, the amino acid mutation in the region of Exon41 may be G1220D, G1241C, G1241V, or G1244D. The “effective amount” means an amount that can improve a symptom or condition to be treated, prevented, or improved. As the symptom or condition to be treated, prevented, or improved, for example, disappearance of α5(IV) expression on the basement membrane; widespread expression of α2(IV) in GBM; increased ectopic expression of laminin; proteinurina; leakage of urinary albumin; elevated serum creatinine levels; and other progressive renal conditions can be mentioned.

In the present specification, the patient to be the subject of treatment, prophylaxis, or improvement includes mammals such as human, cow, horse, dog, cat, pig, sheep and the like, and is preferably human.

The medical composition of the present invention can be administered in vivo, ex vivo, or in vitro.

Furthermore, the present invention relates to the use of a cyclophilin D inhibitor for the production of a therapeutic, prophylactic, or improving drug for AS. The present invention relates to the use of a cyclophilin D inhibitor for the production of a medicament that improves deficiency in the secretion of collagen trimers. The present invention relates to the use of a cyclophilin D inhibitor for the production of an agent for promoting secretion of collagen trimers. Also, the present invention relates to a cyclophilin D inhibitor for the treatment, prophylaxis, or improvement of AS. The present invention relates to a cyclophilin D inhibitor for improving deficiency in the secretion of collagen trimers. The present invention relates to a cyclophilin D inhibitor for promoting the secretion of collagen trimers. For example, when a cyclophilin D inhibitor is used for the purpose of treatment, prophylaxis, or improvement, a medical composition containing the cyclophilin D inhibitor as an active ingredient can be administered in an oral administration form or a parenteral administration form, such as injection, drip transfusion and the like. When a cyclophilin D inhibitor is administered to a mammal or the like, the aforementioned preparation may be orally administered, or may be administered parenterally as an injection or a drip transfusion. The dose varies depending on symptoms, age, gender, body weight, administration form, and the like. For example, when orally administered to an adult, the daily dose is generally 0.1-1000 mg.

In another embodiment, the present invention relates to a non-human model animal having a G1220D mutation in the COL4A5 gene, or a non-human model animal in which Exon41 in the COL4A5 gene is deleted or disrupted. The deletion of Exon 41 is not necessarily in the entire region and may be a partial deletion of Exon 41 as long as it exhibits symptoms based on the deficiency in the secretion of collagen trimers. Alternatively, the gene in Exon 41 may be disrupted. For example, a non-human model animal may be characterized by having one or more of the following phenotypes selected from the following, as compared with the corresponding wild type animal: disappearance of α5(IV) expression on the basement membrane; widespread expression of α2(IV) in GBM; increased ectopic expression of laminin; proteinurina; leakage of urinary albumin; elevated serum creatinine levels; and other progressive renal conditions. The symptoms based on the deficiency in the secretion of collagen trimers may be of the phenotypes listed above.

Alternatively, in another embodiment, the present invention may be directed to a nucleic acid construct in which Exon 41 is deleted or disrupted. The construct can be a nucleic acid construct for producing a model animal of the present invention or an agent for generating a model animal of the present invention.

The non-human model animal does not need to be an adult and may be its cell, tissue, or embryo. The non-human model animal may be a mouse, rat, guinea pig, hamster, rabbit, dog, pig, sheep, cow, or monkey, and is preferably a mouse.

The present invention includes an evaluation method or a screening method for the therapeutic effect of the aforementioned drug on AS using the non-human model animal. Specifically, this method may be a method including administering a test drug to the aforementioned non-human animal model, observing or measuring symptoms or conditions based on the deficiency in the secretion of collagen trimers, or levels (disappearance of α5(IV) expression on the basement membrane; widespread expression of α2(IV) in GBM; increased ectopic expression of laminin; proteinurina; leakage of urinary albumin; elevated serum creatinine levels; and other progressive renal conditions) to be the indices thereof in the aforementioned non-human model animal, and, when the administration of the test drug improves the symptoms or conditions based on the deficiency in the secretion of collagen trimers, or levels to be the indices thereof, determining that the test drug may have a therapeutic effect on AS. That it “improves the symptoms or conditions based on the deficiency in the secretion of collagen trimers, or levels to be the indices thereof” means that the condition becomes closer to that in the wild type than in the model animal, and does not have to mean that the symptom or condition is completely eliminated.

The present invention is explained in more detail in the following by referring to Examples which do not limit the scope of the present invention. Documents cited throughout the present specification are incorporated in full in the present specification by reference.

### EXAMPLE

### (Example 1) Synthesis of NIM258

### (1) Synthesis of tert-butyl(furan-2-yl(tosyl)methyl)carbamate (4)

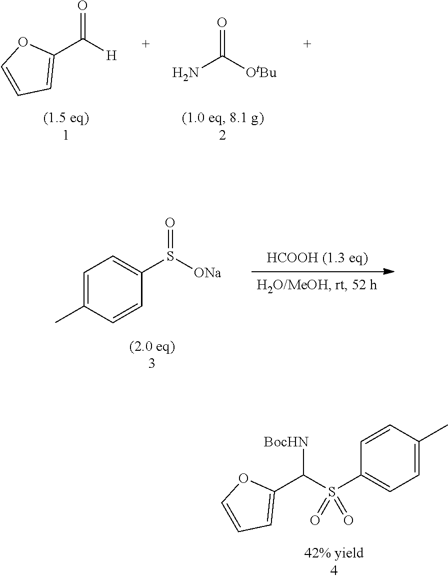

To a suspension prepared from carbamate (2) (8.13 g, 69.4 mmol) and sulfinic acid 3 (35.2 g, 140 mmol, 2.0 eq), and a mixed solvent (2/1, 168 mL) of methanol and water were added aldehyde 1 (8.65 mL, 104.1 mmol, 1.5 eq) and formic acid (3.4 mL, 90.2 mmol, 1.3 eq). The mixture was stirred at room temperature for 52 hr, and a precipitated white solid was collected by filtration. The solid was washed with water and diethyl ether, and dried by heating under reduced pressure to give carbamate (4) (10.2 g, 42% yield).

### (2) Synthesis of tert-butyl((1S,2S)-1-(furan-2-yl)-2-methyl-3-oxopropyl)carbamate (7)

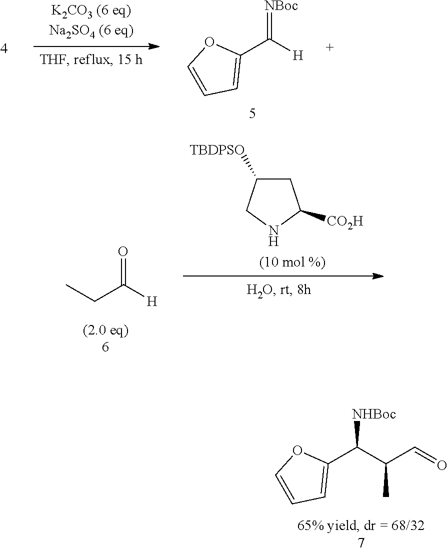

Anhydrous sodium sulfate (53.0 g) and calcium carbonate (15.4 g) were measured into a two-neck flask, and dried under reduced pressure with a heat gun. After cooling to room temperature, anhydrous THF (100 mL) was added, to the suspension was added carbamate 4 (6.6 g, 18.6 mmol), and the mixture was heated under reflux for 15 hr. After cooling to room temperature, the suspension was passed through celite to remove solids, and the filtrate was evaporated under reduced pressure to give imine 5 (3.9 g) as a yellow liquid. The obtained product was used for the next step without further purification.

Under argon atmosphere and at room temperature, water (11 mL) was added to imine 5 (3.6 g, 18.6 mmol), aldehyde 6 (2.16 g, 37.2 mmol, 2.0 eq) and (2S,4R)-4-((tert-butyldiphenylsilyl)oxy)pyrrolidine-2-carboxylic acid (689.7 mg, 1.86 mmol, 10 mol %) were added, and the reaction solution was stirred for 8 hr. The reaction solution was diluted with water, and the aqueous layer was extracted three times with ethyl acetate (30 mL). The combined organic layer was washed with saturated brine, and dried over anhydrous sodium sulfate. The solvent was evaporated under reduced pressure, and the obtained crude product was purified by silica gel column chromatography (hexane/ethyl acetate=12/1) to give the desired aldehyde 7 (3.07 g, 65% yield, dr 68/32) as a diastereomer mixture.

### (3) Synthesis of tert-butyl((1S,2S)-1-(furan-2-yl)-3-hydroxy-2-methylpropyl)carbamate (8)

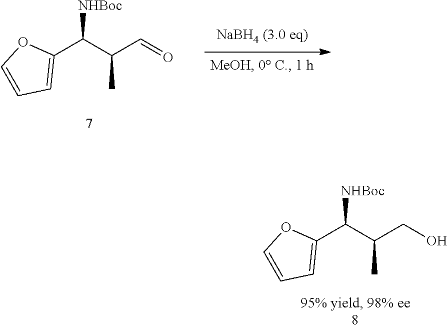 [SMILES: C[C@H](C=O)[C@H](NC(=O)OC(C)(C)C)c1ccco1 ; C[C@H](CO)[C@H](NC(=O)OC(C)(C)C)c1ccco1]

To a methanol solution (40 mL) of aldehyde 7 (3.07 g, 12.1 mmol) was slowly added under ice-cooling sodium borohydride (1.38 g, 36.4 mmol, 3.0 eq). After stirring for 1 hr, a saturated ammonium chloride aqueous solution was added to discontinue the reaction. The reaction solution was extracted three times with ethyl acetate. The combined organic layer was washed with saturated brine, and dried over anhydrous sodium sulfate. The solvent was evaporated and the obtained crude product was purified by silica gel column chromatography (hexane/ethyl acetate=7/1) to give alcohol 8 (2.96 g, 95% yield).

### (4) Synthesis of tert-butyl((1S,2S)-3-((tert-butyldiphenylsilyl)oxy)-1-(furan-2-yl)-2-methylpropyl)carbamate (9)

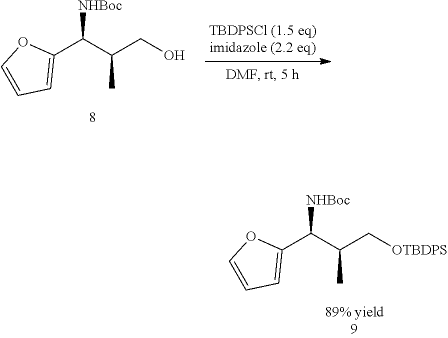

To N,N-dimethylformamide solution (45 mL) in which alcohol 8 (2.96 g, 11.6 mmol) was dissolved were successively added, under argon atmosphere and at room temperature, tert-butyldiphenylsilyl chloride (4.7 mL, 17.1 mmol, 1.5 eq) and imidazole (1.75 g, 25.5 mmol, 2.2 eq), and the mixture was stirred at room temperature for 5 hr. The reaction solution was ice-cooled, and 1N aqueous hydrochloric acid solution (15 mL) was slowly added to discontinue the reaction. The mixture was heated to room temperature, further stirred for 30 min, and extracted three times with ethyl acetate/hexane mixed solution (2/3, 30 mL). The combined organic layer was washed with saturated aqueous sodium hydrogen carbonate and saturated brine in this order, dried over anhydrous sodium sulfate, and the solvent was evaporated under reduced pressure. The obtained crude product was purified by silica gel column chromatography (hexane/ethyl acetate=19/1) to give carbamate 9 (5.11 g 89% yield) as a diastereomer mixture.

### (5) Synthesis of tert-butyl((1S,2S)-3-((tert-butyldiphenylsilyl)oxy)-1-(furan-2-yl)-2-methylpropyl)(methyl) carbamate (10)

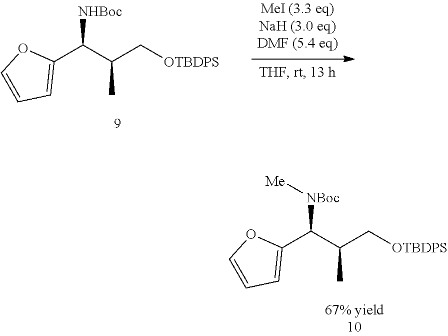

Under an argon atmosphere, anhydrous THF (30 mL) and sodium hydride (60%, 1.24 g, 31.0 mmol) were successively added to a 200 mL eggplant flask. After cooling to 0° C., to the reaction mixture was added a THF solution (10 mL) of carbamate 9 (5.11 g, 10.3 mmol) with a cannula. After stirring at 0° C. for 15 min, methyl iodide (2.13 mL, 34.2 mmol, 3.3 eq) and N,N-dimethylformamide (4.33 mL, 55.9 mmol, 5.4 eq) were successively added. The reaction solution was returned to room temperature and stirred for 13 hr. A saturated ammonium chloride aqueous solution was added to discontinue the reaction, and the aqueous layer was extracted three times with ethyl acetate (20 mL). The combined organic layer was washed with saturated brine, dried over anhydrous sodium sulfate, and the solvent was evaporated under reduced pressure to give a crude product. This was purified by silica gel column chromatography (hexane/ether=12/1) to give carbamate 10 (3.53 g, 67% yield).

### (6) Synthesis of (1S,2S)-3-((tert-butyldiphenylsilyl)oxy)-1-(furan-2-yl)-N,2-dimethylpropane-1-amine (11)

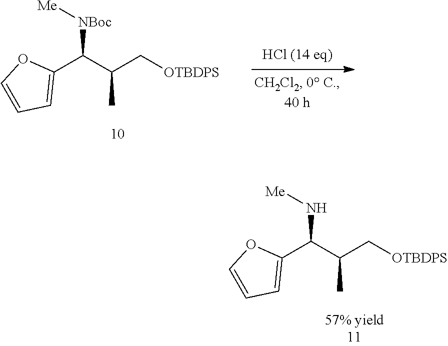

Under an argon atmosphere and at 0° C., to a dichloromethane solution (20 mL) of carbamate 10 (2.91 g, 5.73 mmol) was added hydrogen chloride/dioxane solution (4.0 M, 20 mL), and the mixture was stirred for 45 hr. Saturated aqueous sodium hydrogen carbonate (20 mL) was added to discontinue the reaction, and the aqueous layer was extracted 6 times with dichloromethane (15 mL). The combined organic layer was washed with saturated brine, dried over anhydrous sodium sulfate, and the solvent was evaporated under reduced pressure to give a crude product. This was purified by silica gel column chromatography (hexane/ethyl acetate=5/1-4/1) to give amine 11 (1.33 g, 57% yield).

### (7) Synthesis of tert-butyl(R)-1-((((1S,2S)-3-((tert-butyldiphenylsilyl)oxy)-1-(furan-2-yl)-2-methylpropyl)(methyl)amino)-1-oxopropan-2-yl)(methyl)carbamate (13)

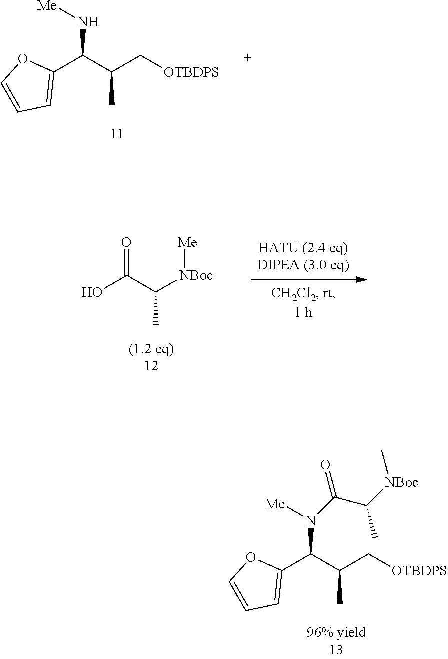

Under an argon atmosphere, amine 11 (781 mg, 1.92 mmol) and carboxylic acid 12 (469 mg, 2.30 mmol) were dissolved in anhydrous dichloromethane (18 mL). After cooling to 0° C., HATU (1.75 g, 4.60 mmol) and N,N-diisopropylethylamine (1.0 mL, 5.75 mmol) were added. The mixture was heated to room temperature, and further stirred for 1 hr. The solution was diluted with ethyl acetate (20 mL) and water (5 mL), and 1N hydrochloric acid (10 mL) was added to discontinue the reaction. The aqueous layer was extracted three times with ethyl acetate (15 mL), and the combined organic layer was washed with saturated aqueous sodium hydrogen carbonate and saturated brine, and dried over anhydrous sodium sulfate. The solvent was evaporated under reduced pressure and the obtained crude product was purified by silica gel column chromatography (hexane/ethyl acetate=9/1-8/1) to give amide 13 (1.09 g, 96% yield).

### (8) Synthesis of (2S,3S)-2-((R)-2-((tert-butoxycarbonyl)(methyl)amino)-N-methylpropanamide)-4-((tert-butyldiphenylsilyl)oxy)-3-methylbutanic acid (14)

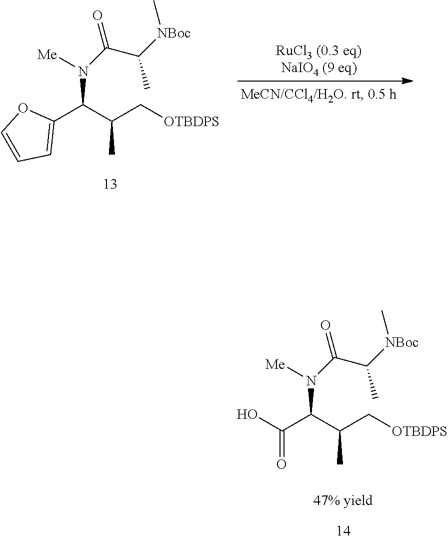

To an acetonitrile/carbon tetrachloride/water suspension (22 mL/10 mL/22 mL) of sodium periodate (3.50 g, 16.4 mmol, 9.0 eq) was added chloride ruthenium (III) (113.5 mg, 0.55 mmol) with vigorous stirring, and the mixture was further stirred for 15 min. To the reaction mixture was added a carbon tetrachloride solution (5 mL) of amide 13 (1.08 g, 1.82 mmol) with a cannula. After stirring for 30 min, water and 10% sodium thiosulfate aqueous solution were added to discontinue the reaction. The aqueous layer was extracted four times with ethyl acetate (30 mL), and the combined organic layer was washed with saturated brine and dried over anhydrous sodium sulfate. The solvent was evaporated and the obtained crude product was purified by silica gel column chromatography (hexane/ethyl acetate) to give carboxylic acid 14 (533.4 mg, purity 90%). The obtained desired product was used for the next step without further purification.

### (9) Synthesis of benzyl(2S,3S)-2-((R)-2-((tert-butoxycarbonyl)(methyl)amino)-N-methylpropanamide)-4-((tert-butyldiphenylsilyl)oxy)-3-methylbutanoate (15)

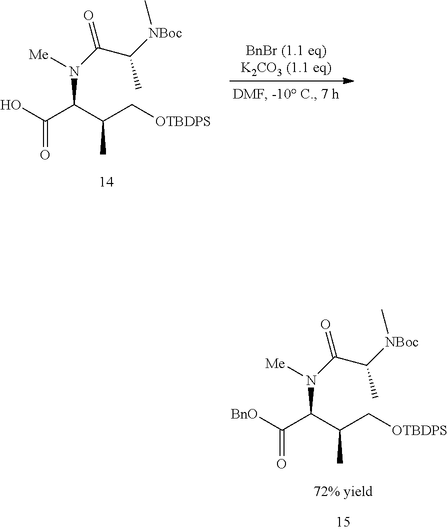

Under an argon atmosphere and at −10° C., to an N,N-dimethylformamide solution (10 mL) of carboxylic acid 14 (528.9 mg, 90% purity, 0.93 mmol) were slowly added potassium carbonate (141.1 mg, 1.02 mmol) and benzyl bromide (0.12 ml, 1.02 mmol). After 7 hr, water was added to discontinue the reaction, and the aqueous layer was extracted three times with ethyl acetate (20 mL). The combined organic layer was washed with saturated brine, dried over anhydrous sodium sulfate, and the solvent was evaporated. The obtained crude product was purified by silica gel column chromatography (hexane/dichloromethane=1/2) to give benzyl ester 15 (442.1 mg, 72% yield).

### (10) Synthesis of benzyl(2S,3S)-2-((R)-2-((tert-butoxycarbonyl)(methyl)amino)-N-methylpropanamide)-4-hydroxy-3-methylbutanoate (16)

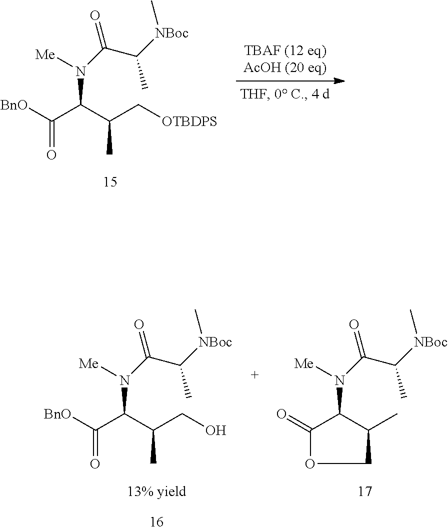

Under an argon atmosphere and at 0° C., to a THF solution (25 mL) of ester 15 (304.8 mg, 0.46 mmol) was added acetic acid (0.53 mL, 9.2 mmol, 20 eq). To this solution was added tetrabutylammonium fluoride (5.5 mL, 1.0 M in THF, 12 eq), and the mixture was stirred for 4 days. The mixture was diluted with ethyl acetate and saturated brine, and the aqueous layer was extracted four times with ethyl acetate (15 mL). The combined organic layer was washed with saturated brine, dried over anhydrous sodium sulfate, and the solvent was evaporated under reduced pressure to give a crude product. The obtained compound was purified by silica gel column chromatography (hexane/ethyl acetate=5/1-1/1) to give alcohol 16 (48.5 mg, purity 50%, containing lactone 17, 13% yield).

### (11) Synthesis of benzyl(2S,3S)-2-((R)-2-((tert-butoxycarbonyl)(methyl)amino)-N-methylpropanamide)-3-methyl-4-oxobutanoic acid (18)

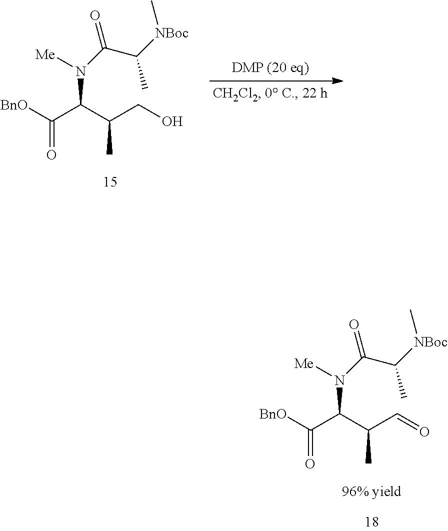 [SMILES: C[C@H](CO)[C@@H](C(=O)OCc1ccccc1)N(C)C(=O)[C@@H](C)N(C)C(=O)OC(C)(C)C ; C[C@H](C=O)[C@@H](C(=O)OCc1ccccc1)N(C)C(=O)[C@@H](C)N(C)C(=O)OC(C)(C)C]

Under an argon atmosphere and at 0° C., to a dichloromethane solution (10 mL) of alcohol 16 (48.5 mg, 50% purity, 0.115 mmol) was added a Dess-Martin reagent (1.01 g, 2.3 mmol), and the mixture was stirred for 22 hr. A 10% sodium thiosulfate aqueous solution (8 mL) and saturated aqueous sodium hydrogen carbonate (8 mL) were added to discontinue the reaction, and the mixture was diluted with ethyl acetate (20 mL). After stirring for 20 min, the aqueous layer was extracted three times with ethyl acetate (10 mL), and the combined organic layer was washed with saturated brine, and dried over anhydrous sodium sulfate. The solvent was evaporated and the obtained crude product was purified by silica gel column chromatography (hexane/ethyl acetate=4/1) to give aldehyde 18 (23.3 mg, 96% yield).

### (12) Synthesis of benzyl(2S,3R)-2-((R)-2-((tert-butoxycarbonyl)(methyl)amino)-N-methylpropanamide)-4-(4-(2-methoxyethyl)piperazin-1-yl)-3-methylbutanoate (19)

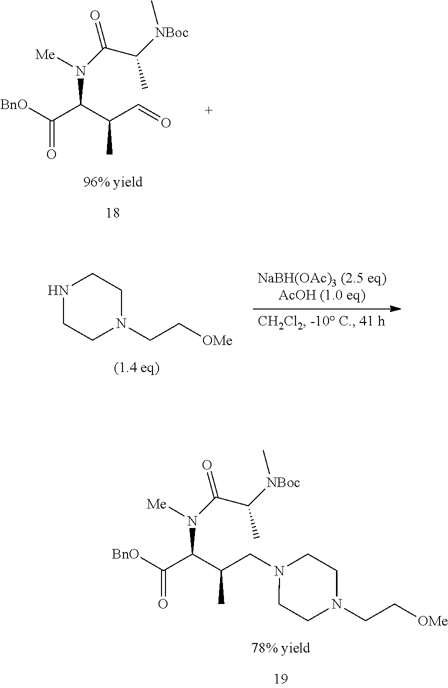

Under an argon atmosphere and at −20° C., to a dichloromethane solution (2 mL) of aldehyde 18 (23.6 mg, 0.056 mmol) were successively added acetic acid (3.5 mg, 0.056 mmol) and 1-(2-methoxyethyl)piperazine (11.4 mg, 0.079 mmol). To this solution was slowly added sodium triacetoxyborohydride (29.9 mg, 0.14 mmol) and the mixture was stirred at −10° C. for 41 hr. The reaction was discontinued with saturated ammonium chloride aqueous solution, and the aqueous layer was extracted 5 times with ethyl acetate (10 mL). The combined organic layer was washed with saturated brine, dried over anhydrous sodium sulfate, and the solvent was evaporated under reduced pressure to give a crude product. This was purified by silica gel column chromatography (2-7% methanol/dichloromethane) to give amine 19 (24.0 mg, 78% yield).

### (13) Synthesis of (2S,3R)-2-((R)-2-((tert-butoxycarbonyl)(methyl)amino)-N-methylpropanamide)-4-(4-(2-methoxyethyl)piperazin-1-yl)-3-methylbutanoic acid

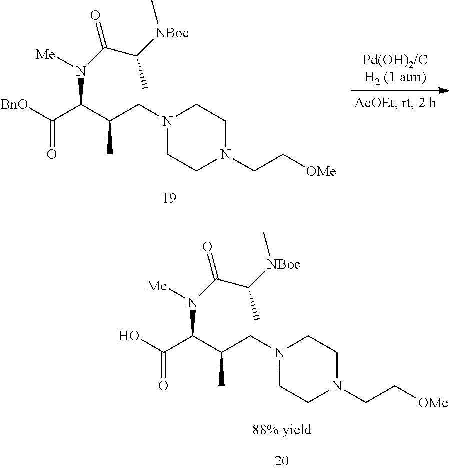 [SMILES: COCCN1CCN(C[C@@H](C)[C@@H](C(=O)OCc2ccccc2)N(C)C(=O)[C@@H](C)N(C)C(=O)OC(C)(C)C)CC1 ; COCCN1CCN(C[C@@H](C)[C@@H](C(=O)O)N(C)C(=O)[C@@H](C)N(C)C(=O)OC(C)(C)C)CC1]

To an ethyl acetate solution (5 mL) of amine 19 (30.8 mg, 0.056 mmol) was added palladium hydroxide (10 wt %, 1.3 mg), and the mixture was stirred under 1 atm hydrogen atmosphere for 2 hr. The reaction solution was passed through celite, the solid was filtered off, and the solvent was evaporated under reduced pressure to give a crude product. This was purified by silica gel column chromatography (7-10% methanol/dichloromethane) to give carboxylic acid 20 (21.4 mg, 83% yield).

### (14) Synthesis of acetyl-CsA (22)

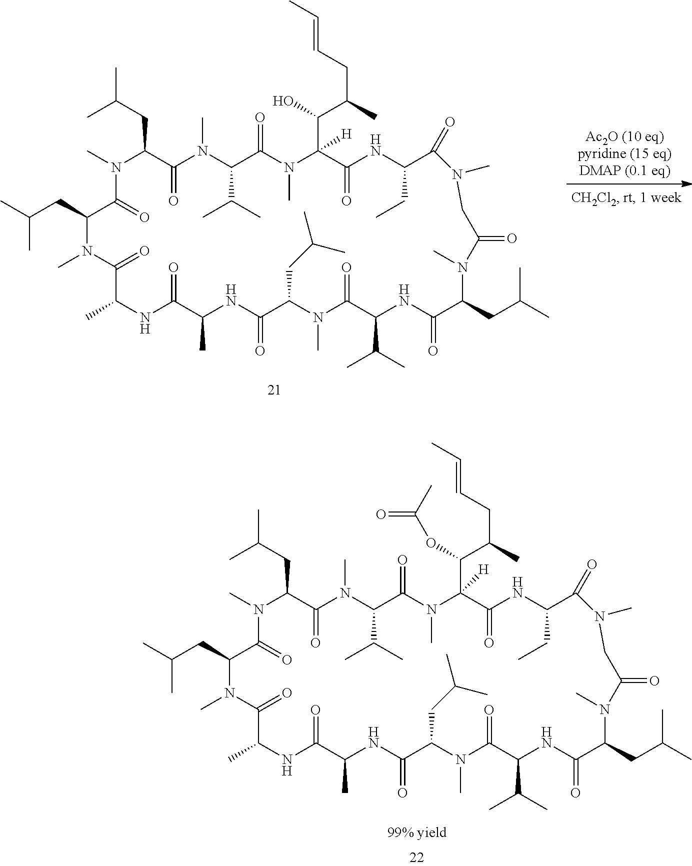

Under an argon atmosphere, cyclosporine A (21) (4.0 g, 3.33 mmol) was dissolved in dichloromethane (24 mL), acetic anhydride (3.2 mL, 33.3 mmol) and pyridine (4.0 mL, 50.0 mmol), and DMAP (40.6 mg, 0.33 mmol, 10 mol %) were successively added. The mixture was heated to room temperature and further stirred for 1 week. Saturated aqueous sodium hydrogen carbonate (100 mL) was slowly poured into the reaction solution. After stirring for 1 hr, the aqueous layer was extracted three times with dichloromethane (20 mL). The combined organic layer was washed two times with 1N hydrogen chloride aqueous solution (48 mL), and with saturated aqueous sodium hydrogen carbonate (48 mL) and saturated brine, and dried over anhydrous sodium sulfate. The solvent was evaporated under reduced pressure to give a crude product (4.1 g). The obtained product was used for the next step without further purification.

### (15) Synthesis of Compound 23

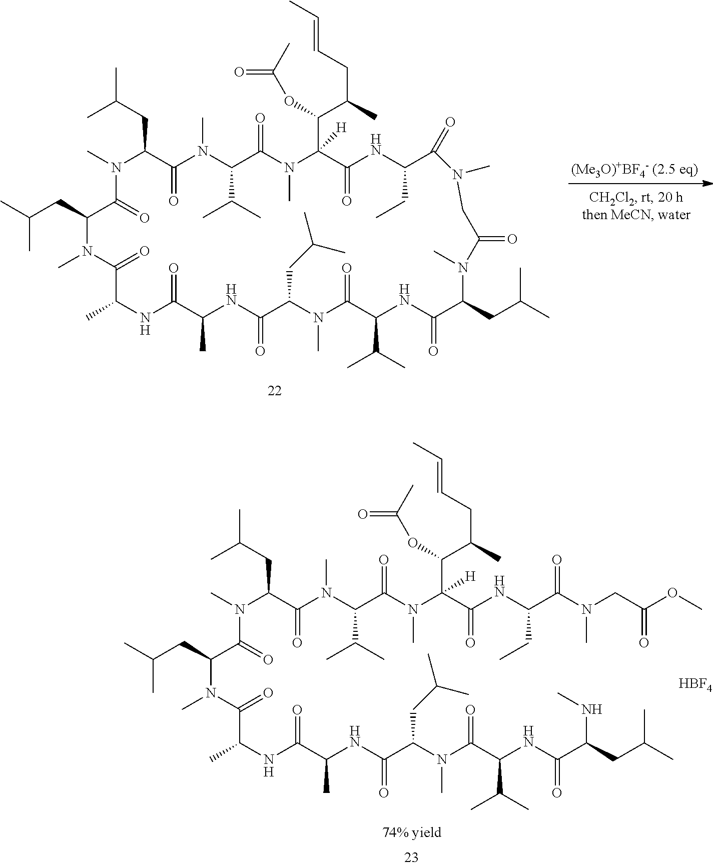

Under an argon atmosphere, to a dichloromethane solution (4 mL) of acetylated compound 22 (2.16 g, 1.74 mmol) was added trimethyloxonium tetrafluoroborate (642.6 mg, 4.34 mmol), and the mixture was stirred at room temperature for 20 hr. To this reaction solution were added acetonitrile (4 mL) and water (13.5 mL), and the mixture was further stirred for 48 hr. The organic layer was extracted and dried over anhydrous sodium sulfate. The solvent was evaporated under reduced pressure to give a crude product. The obtained compound was purified by silica gel flash column chromatography (1-9% methanol/dichloromethane) to give ring-opened compound 23 (1.75 g, 74% yield).

### (16) Synthesis of Compound 24

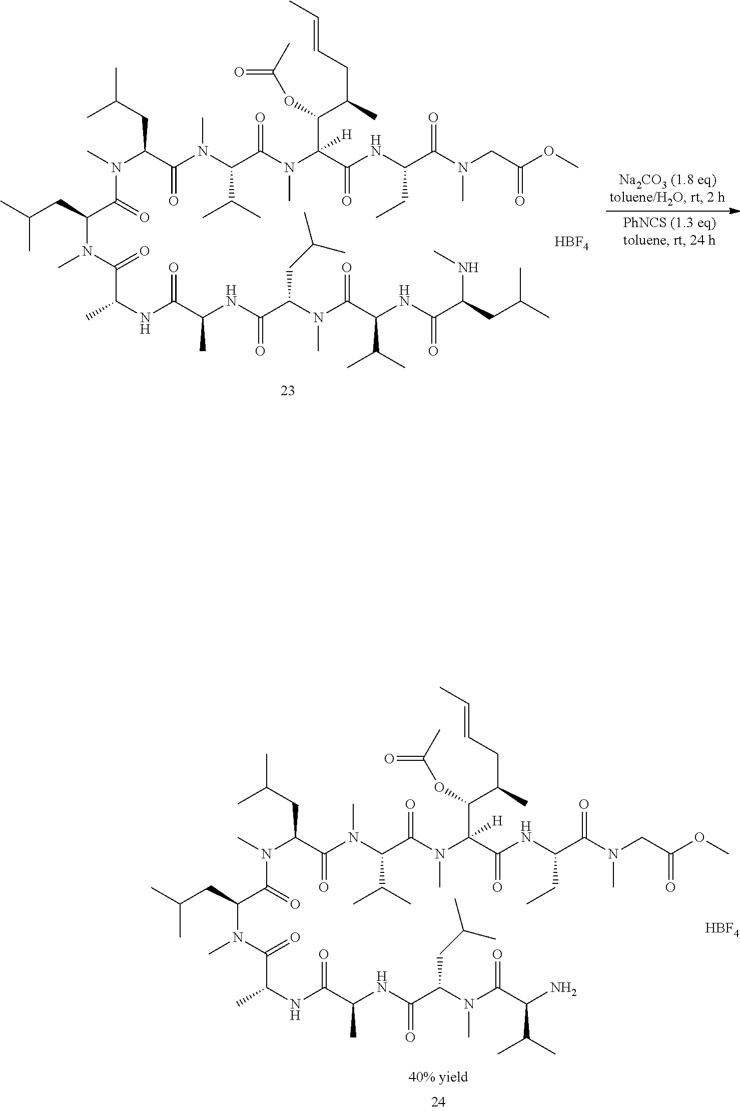

To a mixture of ring-opened compound 23 (1.74 g, 1.28 mmol) in toluene (4.2 mL) and water (2.5 mL) was added sodium carbonate (243 mg, 2.3 mmol) at room temperature. 2-Methyltetrahydrofuran (20 mL) was added to completely dissolve the solid. After stirring for 2 hr, the organic layer was separated and evaporated under reduced pressure. The obtained residue was dissolved in toluene (4.2 mL), and phenylisothiocyanate (224 mg, 1.66 mmol) was added at room temperature. After stirring for 24 hr, methanol (2.4 mL) was added, 50% tetrafluoroboric acid (0.8 mL) was successively added, and the mixture was further stirred for 1 hr. Water (2.4 mL) was added, and the organic layer was recovered by partitioning and washed with water and saturated brine. By drying over anhydrous sodium sulfate and evaporation under reduced pressure, a crude product was obtained and purified by flash column chromatography to give amine 24 (580.6 mg, 40% yield).

### (17) Synthesis of Compound 25

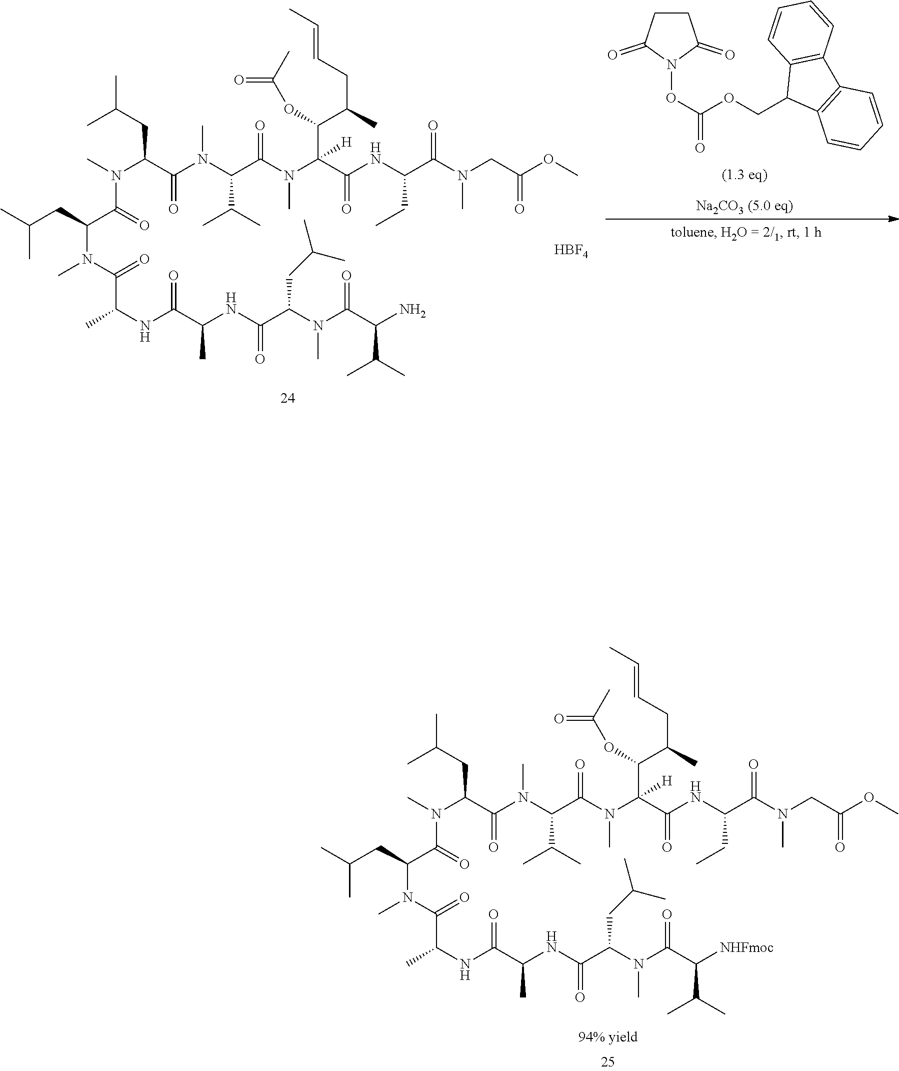

Under an argon atmosphere, to amine 24 (486 mg, 0.39 mmol) were added toluene (2.8 mL) and water (1.5 mL) to prepare a suspension. Sodium carbonate (210 mg, 1.96 mmol) and Fmoc-succinimide (174 mg, 0.51 mmol) were slowly added at room temperature. After stirring for 1 hr, methanol (0.2 mL) was added, and the mixture was further stirred at 50° C. for 1 hr. The organic layer was separated, and the solvent was evaporated under reduced pressure to give a crude product. The obtained compound was purified by silica gel column chromatography (3% methanol/dichloromethane) to give carbamate 25 (508 mg, 94% yield).

### (18) Synthesis of Compound 26

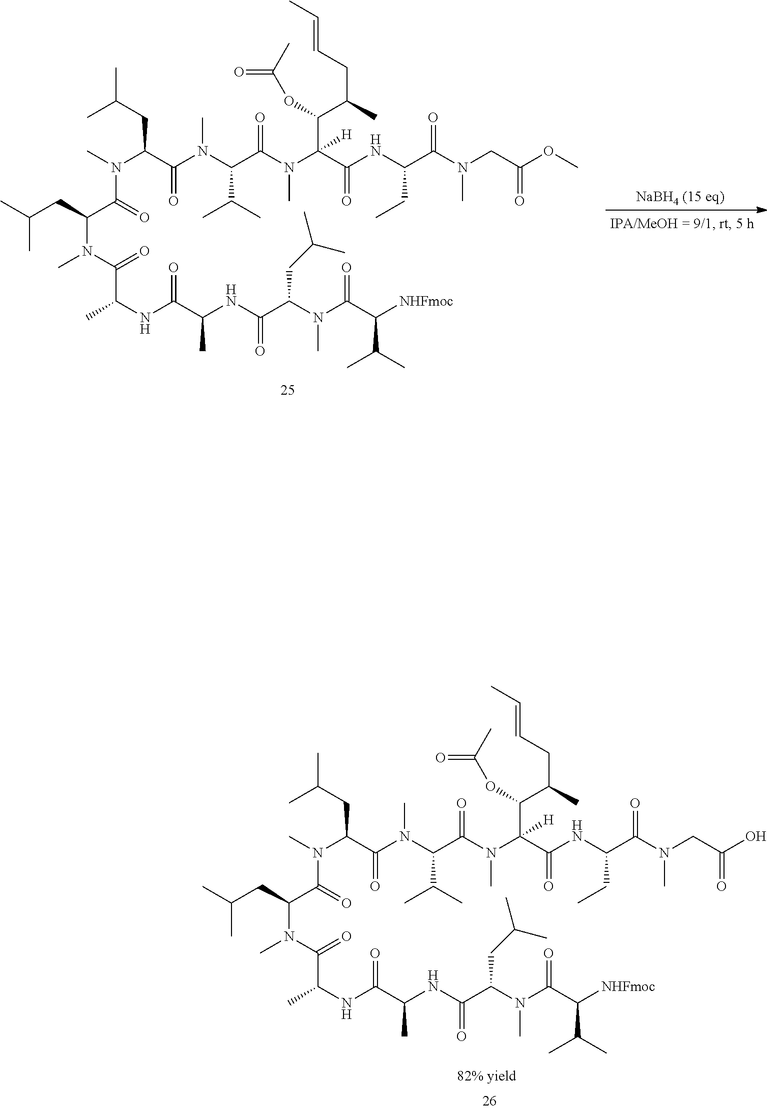

Under an argon atmosphere, to a solution of carbamate 25 (477.1 mg, 0.35 mmol) in isopropyl alcohol (10 mL) and methanol (1.1 mL) was added sodium borohydride (202.3 mg, 5.22 mmol) at 0° C. The mixture was heated to room temperature and further stirred for 5 hr, and water (5 mL) was added to discontinue the reaction. The aqueous layer was extracted 5 times with dichloromethane (10 mL), and the combined organic layer was washed with saturated aqueous sodium hydrogen carbonate and saturated brine, and dried over anhydrous sodium sulfate. The solvent was evaporated under reduced pressure and the obtained crude product was purified by silica gel column chromatography (3% methanol/dichloromethane) to give alcohol 26 (383.7 mg, 82% yield).

### (19) Synthesis of Compound 27

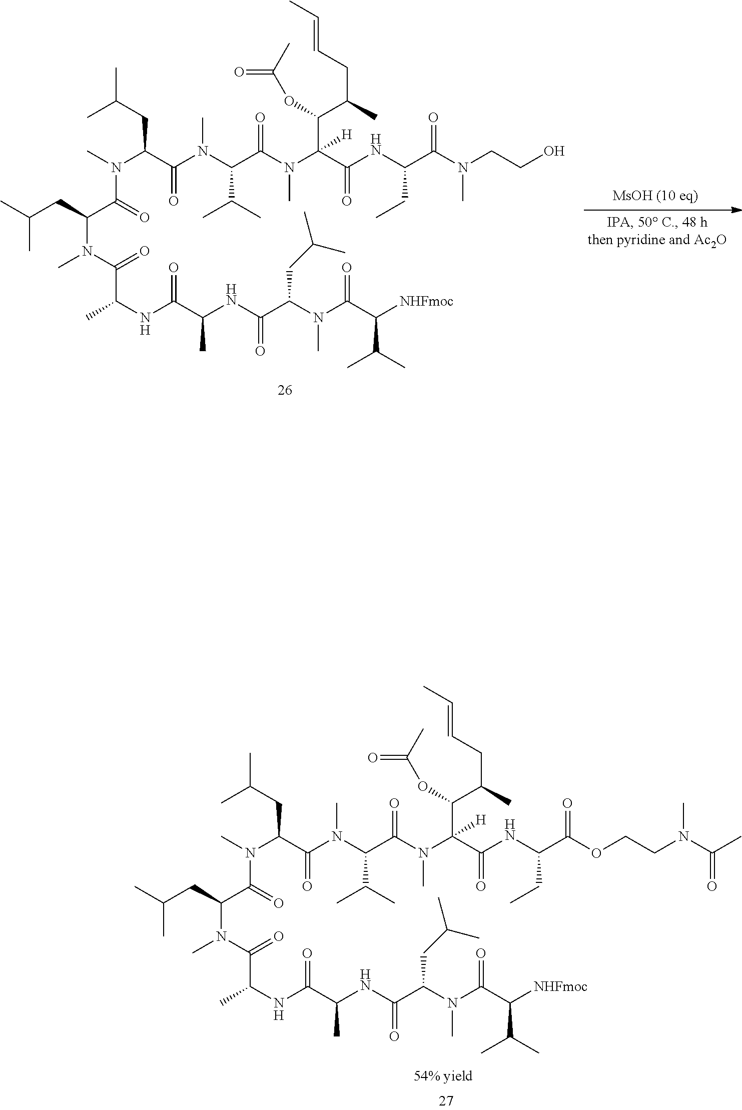

Under an argon atmosphere, alcohol 26 (320.9 mg, 0.24 mmol) was dissolved in isopropyl alcohol (15 mL), methanesulfonic acid (0.16 mL, 2.39 mmol) was added at 50° C., and the mixture was stirred for 48 hr. The reaction solution was cooled to room temperature, and acetic anhydride (0.11 mL, 1.19 mmol) and pyridine (0.48 mL, 5.97 mmol) were successively added. After stirring for 1 hr, saturated aqueous sodium hydrogen carbonate (3 mL) was added to discontinue the reaction. The aqueous layer was extracted four times with dichloromethane (10 mL), and the combined organic layer was washed twice with 5% aqueous hydrochloric acid solution (10 mL), once with saturated aqueous sodium hydrogen carbonate (10 mL), and once with saturated brine. By drying over anhydrous sodium sulfate and evaporation, a crude product was obtained and purified by silica gel column chromatography (1-4% methanol/dichloromethane) to give compound 27 (179.5 mg, 54% yield).

### (20) Synthesis of Compound 28

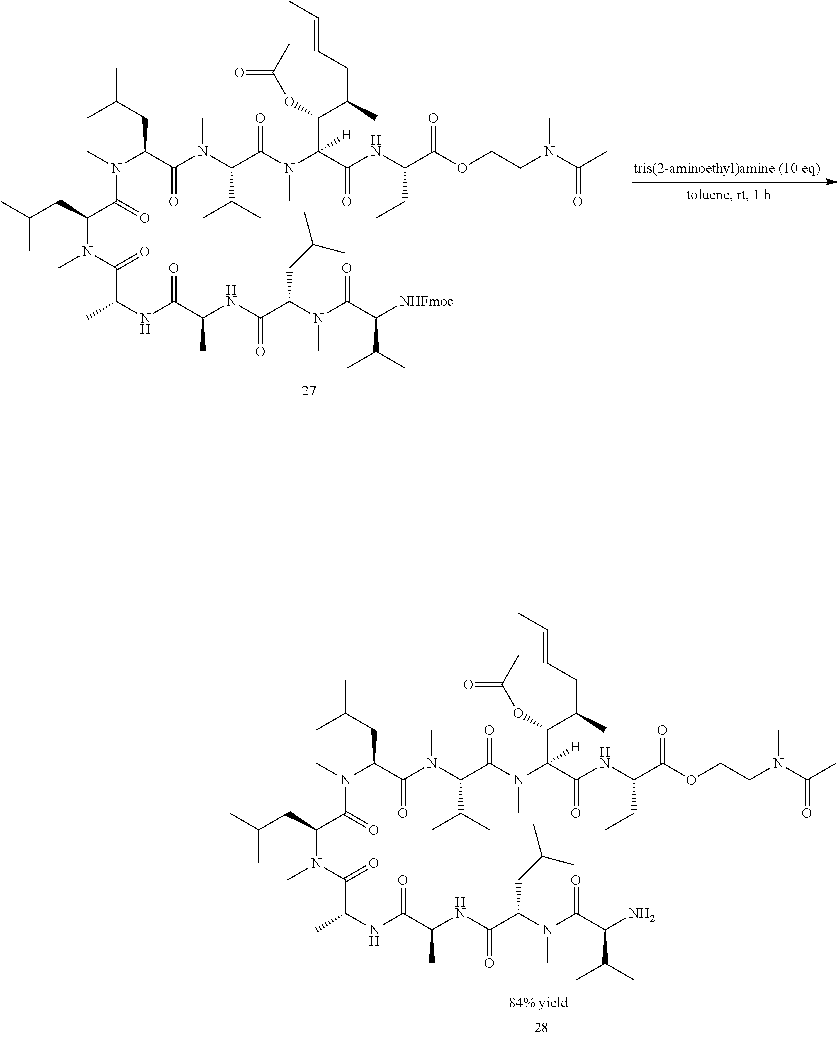

Under an argon atmosphere, to a toluene solution (3 mL) of compound 27 (72.6 mg, 0.052 mmol) was added tris (2-aminoethyl)amine (0.078 mL, 0.52 mmol) at room temperature, and the mixture was stirred for 1 hr. Water (5 mL) was added to discontinue the reaction, and the aqueous layer was extracted three times with dichloromethane (10 mL). The combined organic layer was washed with saturated brine, and dried over anhydrous sodium sulfate. The solvent was evaporated under reduced pressure and the obtained crude product was purified by silica gel column chromatography (5-7% methanol/dichloromethane) to give amine 28 (51.0 mg, 84% yield).

### (21) Synthesis of Compound 29

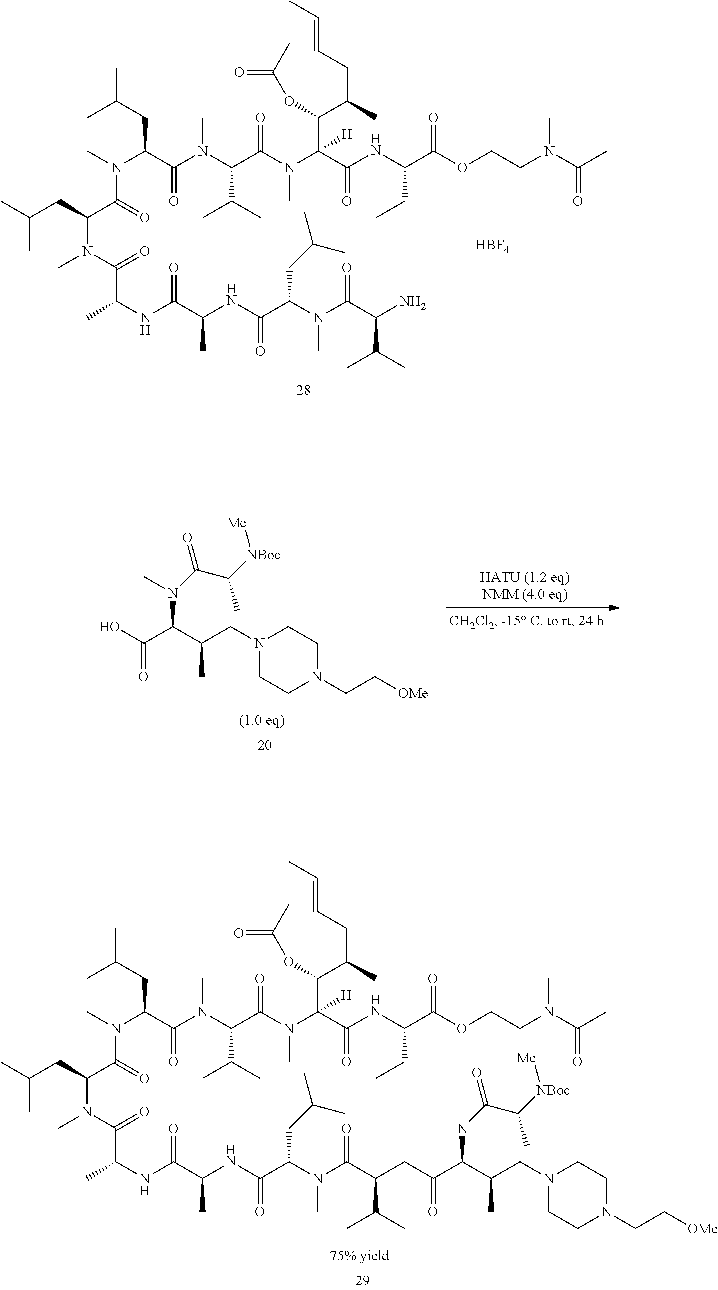

Under an argon atmosphere, amine 28 (26.8 mg, 0.023 mmol) and carboxylic acid 20 (10.6 mg, 0.023 mmol) were dissolved in dichloromethane (3 mL), and a dichloromethane solution of N-methylmorpholine (0.92 mM, 0.1 mL) was added at 0° C. This solution was cooled to −15° C., a DMF solution of HATU (0.28 mM, 0.1 mL) was added, and the mixture was stirred at −15° C. for 24 hr. Saturated ammonium chloride (3 mL) was added to discontinue the reaction, and the aqueous layer was extracted three times with dichloromethane (10 mL). The combined organic layer was washed with saturated brine, and dried over anhydrous sodium sulfate. The solvent was evaporated under reduced pressure and the obtained crude product was purified by silica gel column chromatography (5-10% methanol/dichloromethane) to give a desired compound 29 (27.6 mg, 75% yield).

### (22) Synthesis of NIM258

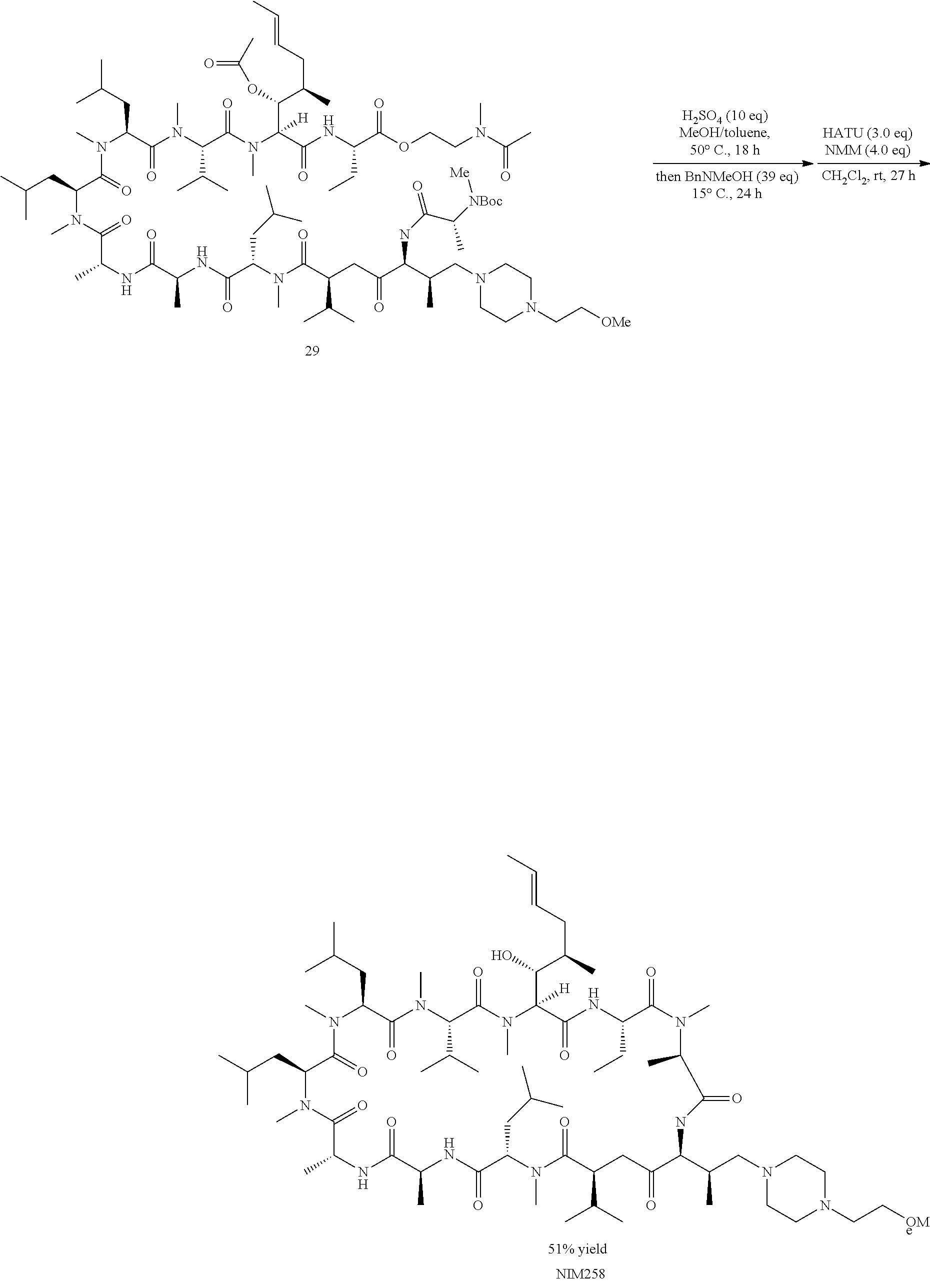

Under an argon atmosphere, compound 29 (27.6 mg, 0.017 mmol) was dissolved in methanol (3 mL) and toluene (1.5 mL), and a methanol solution of sulfuric acid (1.7 mM, 0.2 mL) was added at room temperature. This reaction solution was stirred at 50° C. for 18 hr and cooled to 15° C. A methanol solution of benzyltrimethylammonium hydroxide (6.7 mM, 0.1 mL) was added and the mixture was further stirred for 24 hr. Water (3 mL) was added to discontinue the reaction. After stirring for 4 hr, the aqueous layer was extracted 5 times with dichloromethane (10 mL) and the combined organic layer was washed with water and saturated brine in this order, and dried over anhydrous sodium sulfate. The solvent was evaporated under reduced pressure and the obtained crude product was used for the next step without purification.

Under an argon atmosphere, the crude product (24.8 mg) was dissolved in dichloromethane (180 mL). To this reaction solution was added a dichloromethane solution of HATU (19.8 mg, 0.052 mmol) and N-methylmorpholine (0.23 mM, 0.3 mL) at room temperature, and the mixture was stirred for 27 hr. Thereafter, a saturated ammonium chloride aqueous solution (3 mL) was added to discontinue the reaction, and the aqueous layer was extracted four times with dichloromethane (10 mL). The combined organic layer was washed with saturated brine, and dried over anhydrous sodium sulfate. The solvent was evaporated under reduced pressure and the obtained crudely purified product was purified by silica gel column chromatography (3-10% methanol/dichloromethane) to give a desired NIM258 (11.8 mg, 51% yield).

### (Example 2) Promotion of Collagen Trimer Secretion by CsA, PSC-833, and Alisporivir

### (1) Generation of a Cell Line Stably Expressing Split Nano Luciferase-Fused α345

HEK293T cells (Human Embryonic Kidney 293) were obtained from the RIKEN Cell Bank, and thereafter addition of virus concentration solution for COL4A4-3FLAG (Puro), COL4A3-SmBiT (Hyg), COL4A5-LgBiT (BSD) (Omachi K. et al., Cell Chemical Biology 2018, 25, 634-643) in this order, exchange of drug resistance containing medium, passage and preservation of surviving cells were repeated whereby a cell line stably expressing α345 (IV) trimer was generated. COL4A5 variant G1244D was prepared using pLVSIN EF1α BSD COL4A5-LgBiT as a template and using Quikchange (registered trade mark) II Site-directed Mutagenesis Kit (STRATAGENE (registered trade mark)). The primers used were follows. PCR reactions (95° C. for 1 min, one cycle; 95° C. for 50 sec, 60° C. for 50 sec, 68° C. for 90 sec/1 kb, 18 cycles; 68° C. for 7 sec) were performed with each Primer. The base sequence of DNA was confirmed by the cycle sequence method (Sigma-Aldrich JAPAN).

[G1244D mutation primer]
Sense primer:
(SEQ ID NO: 13)
5′-ccctcctggttctccggatccagctctggaaggacc-3′
Antisense primer:
(SEQ ID NO: 14)
5′-ggtccttccagagctggatccggagaaccaggaggg-3′
[G1241V mutation primer]
Sense primer:
(SEQ ID NO: 15)
5′-gtcccccaggccctcctgtttctccgggtccagctctg-3′
Antisense primer:
(SEQ ID NO: 16)
5′-cagagctggacccggagaaacaggagggcctgggggac-3′

### (2) Luminescence of Type IV Collagen α345 Trimer by Nano Luciferase

A cell line stably expressing split nano luciferase-fused α345 required for the NanoLuc α345 trimer assay was seeded on LumiNuc 96 well white plate (Thermo). After 24 hr, the cells were cultured in phenol red-free DMEM complete medium containing 2-phosphate ascorbic acid (200 μM), substrates were added to the culture supernatant and cells according to the standard method of NanoGlo live cell assay (Promega) reagent, and luminescence was confirmed with luminometer GloMax Navigator (Promega).

### (3) Type IV Collagen α345 Trimer Assay by Nano Luciferase

The cell line stably expressing α345 (COL4A5-G1244D) was detached and seeded on LumiNuc 96 well white plate (Thermo) at a density of 2×10⁴ cells/well. 24 hr after seeding, the medium was exchanged with a phenol red-free DMEM complete medium containing 2-phosphate ascorbic acid (200 μM) and each concentration of cyclosporin A (CsA) (0.1, 0.5, 1, 2, 5, 10 μM), or each concentration of PSC-833 (0.5, 1, 2, 5, 10 μM), or each concentration of Alisporivir (0.1, 0.5, 5, 10 μM) and the cells were cultured. A substrate was added 24 hr later to the culture supernatant according to the standard method of NanoGlo live cell assay (Promega) reagent, and luminescence (index of trimer secretion) was measured by luminometer GloMax Navigator (Promega). The control solvent (CON) used was dimethyl sulfoxide (DMSO).

### (4) Results

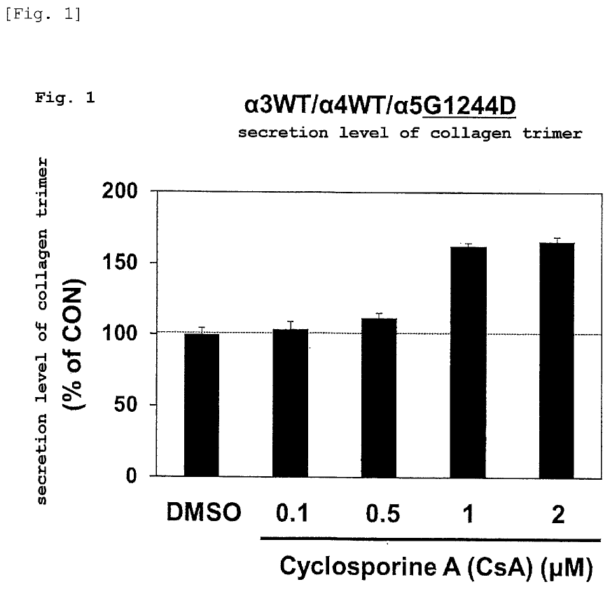

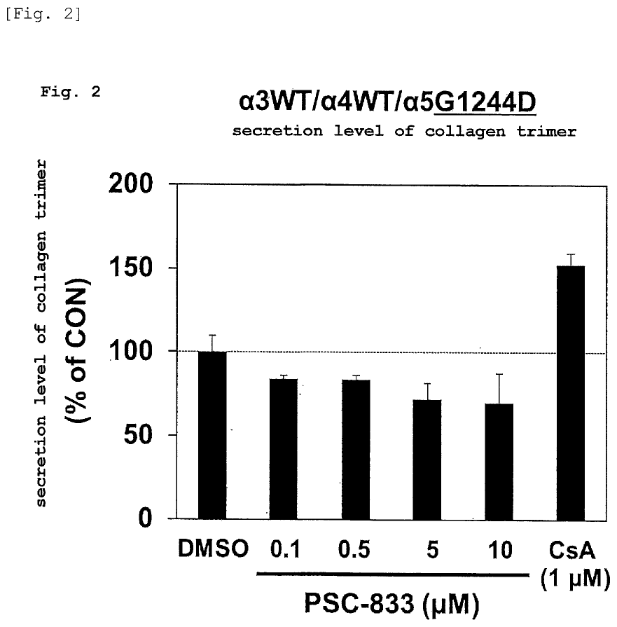

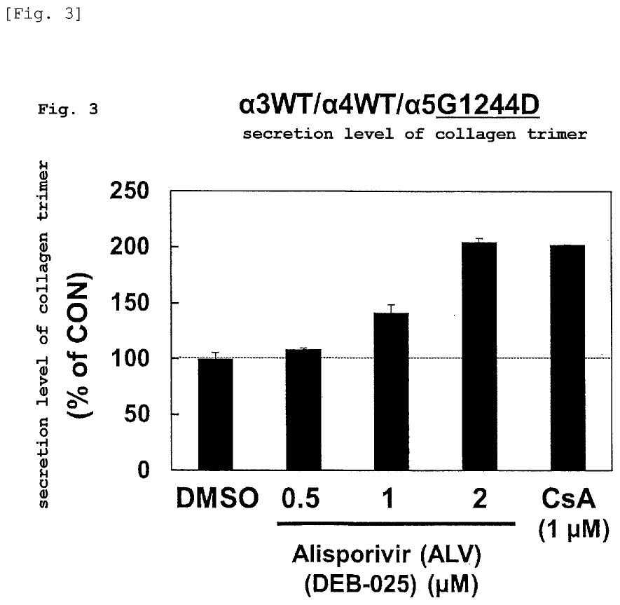

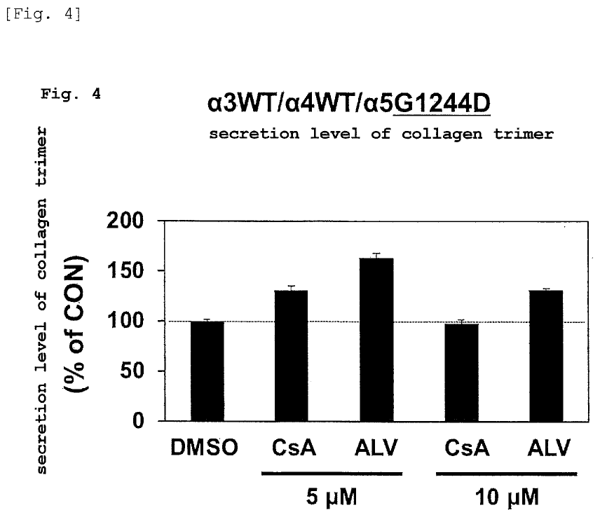

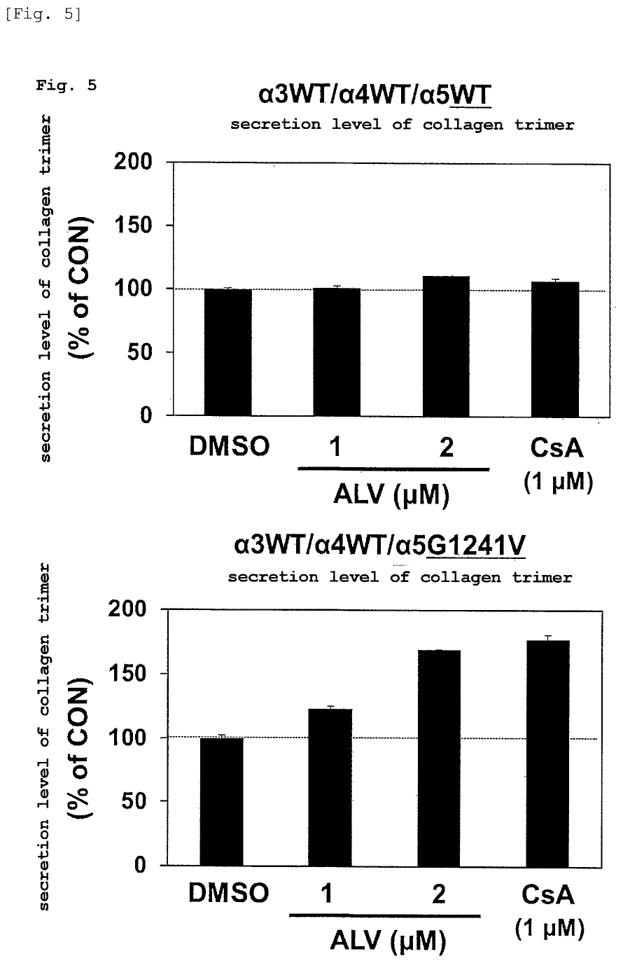

The results are shown in FIG. 1-FIG. 4. The proportion of luminescence (trimer secretion) in the drug-treated group compared to the DMSO-treated group (control: CON) was calculated and expressed in %. CsA was used as the positive control in FIG. 2 and FIG. 3. As a result, CsA promoted collagen trimer secretion, but PSC-833 did not affect collagen trimer secretion or intracellular expression level. On the other hand, Alisporivir showed an action of promoting collagen trimer secretion. In addition, under the treatment conditions of concentrations of 5 μM and 10 μM, Alisporivir showed a stronger action of promoting collagen trimer secretion than CsA.

### (Example 3) Relationship Between Collagen Mutation and Promotion of Secretion Level by Drug

As has been reported to involve various gene mutations. It has been reported that COL4a5/G1241V, like G1244D, maintains relatively high intracellular formation but shows reduced trimer secretion. On the other hand, COL4a5/WT in healthy subjects who do not develop AS is normal in both intracellular formation and trimer secretion. Therefore, the influence of CsA and Alisporivir on the secretion of trimers of COL4a5/WT and COL4a5/G1241V-containing collagens was investigated by an evaluation system using a homeostatic expression cell line.

As a result, CsA and Alisporivir did not show an influence on COL4a5/WT. On the other hand, CsA and Alisporivir promoted extracellular secretion of collagen trimers for COL4a5/G1241V, like G1244D. Therefore, it was shown that CsA and Alisporivir have no action on normal cells and have an effect of promoting the secretion of trimers of mutant COL4a5-containing collagen.

### (Example 4) Collagen Trimer Secretion when NIM258 is Administered

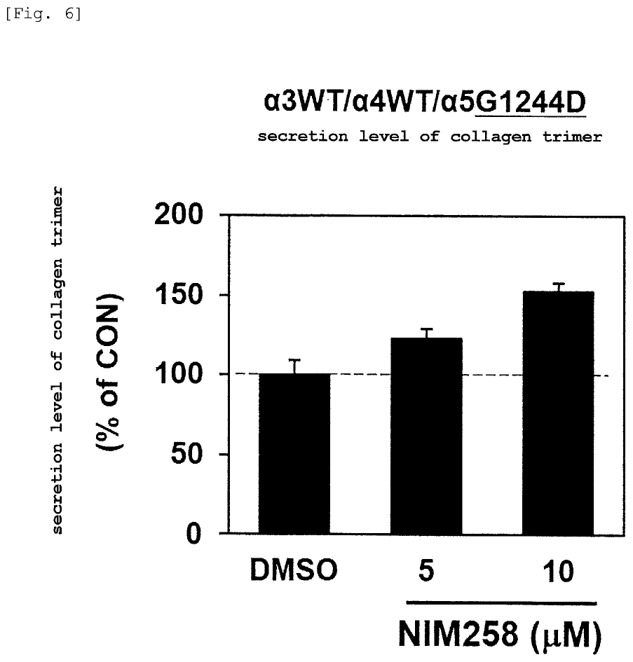

By a method similar to that in Example 1 and Example 2, the extracellular secretion of collagen trimer when 5 μM or 10 μM of NIM258 was administered was measured. As a result, the extracellular secretion of collagen trimers was promoted in the NIM258-administered group as compared with the control DMSO-administered group (FIG. 6). Therefore, it was shown that NIM258 has an effect of promoting the secretion of trimers of mutant COL4a5-containing collagen, like CsA and Alisporivir.

### (Example 5) Relationship Between Various Collagen Mutations in COL4A5 and Effectiveness of CsA and Alisporivir

A wide range of AS mutants (G227S, G594D, G624D, G869R, S916G, G1030S, G1107R, G1140V, G1220D, G1241C, G1241V, G1244D, P1517T, and R1569Q of COL4A5) with clinical reports, and Exon41 (1202nd to 1263rd amino acids of COL4a5)-deleted variant were measured for the trimer secretion promoting ability of CsA and Alisporivir by a method similar to that in Example 1. As the control, wild-type COL4A5 was used.

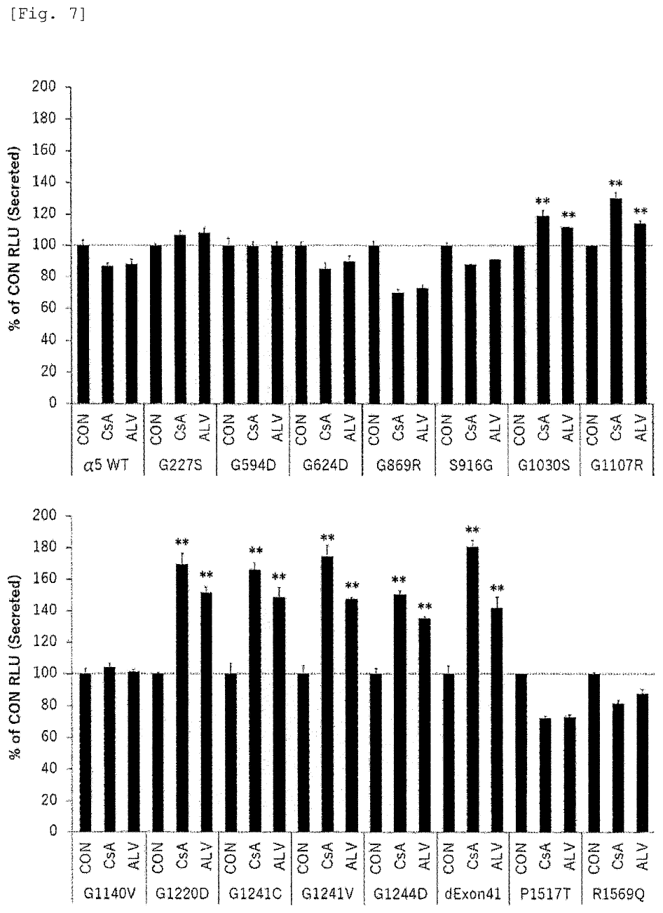

As a result, collagen trimer secretion afforded by CsA and Alisporivir exhibited a significant effect in G1030S, G1107R, G1220D, G1241C, G1241V, G1244D, and Exon41-deleted variants (FIG. 7). In particular, Exon41 deficiency itself and all the mutations existing in Exon41 promoted trimer secretion by CsA and Alisporivir. Therefore, the trimer secretion promoting effect by CsA and Alisporivir is considered to be particularly effective for collagen dysplasia based on the mutations existing in Exon41 and therearound.

### (Example 6) Elucidation of Mechanism of Collagen Trimer Secretion Promotion by CsA and Alisporivir

To elucidate the mechanism of collagen trimer secretion promotion by CsA and Alisporivir, cells in which cyclophilins to which CsA and Alisporivir can bind were knocked down with siRNA were produced, and the collagen trimer secretion promoting effect by CsA and Alisporivir in the cells was investigated comprehensively.

As the cells, G1244D mutation-introduced HEK293T cells—for which the effect of CsA and Alisporivir were confirmed in previous experiments—were used.

An cell line stably expressing split nano luciferase-fused α345 required for the NanoLuc α345 trimer assay was seeded on a 6 well plate (Corning) at 3×10⁵ cells/well, and the siRNAs shown in Table 1 were introduced by lipofection at the time of subconfluence. The target genes are all cyclophilins, and the abbreviations in Table 1 indicate the following substances.

PPIA: Peptidylprolyl isomerase A (cyclophilin A)
PPIB: Peptidylprolyl isomerase B
PPIC: Peptidylprolyl isomerase C
PPID: Peptidylprolyl isomerase D
PPIE: Peptidylprolyl isomerase E
PPIF: Peptidylprolyl isomerase F (cyclophilin D)
PPIG: Peptidylprolyl isomerase G
PPIH: Peptidylprolyl isomerase H
PPIL1: Peptidylprolyl isomerase like 1

### NKTR: Natural Killer Cell Triggering Receptor (Peptidyl-Prolyl Cis-Trans Isomerase NKTR)

### PPWD1: Peptidylprolyl Isomerase Domain and WD Repeat Containing 1

Lipofectamine RNAi Max was used for transfection of siRNA. The final siRNA concentration was adjusted to 15 nM, and transfection was performed according to the standard protocol. 24 hr after siRNA introduction, the cells were detached and reseeded on a LumiNuc 96 well white plate (Thermo) at 4×10⁴ cells/well. 24 hr thereafter, the medium was exchanged with a phenol red-free DMEM complete medium containing 2-phosphate ascorbic acid (200 μM) and Cyclosporin A (CsA) (1 μM), or Alisporivir (2 μM) and the cells were cultured. Substrates were added 24 hr later to the culture supernatant and cells according to the standard method of NanoGlo live cell assay (Promega) reagent, and luminescence (index of trimer secretion) was measured by luminometer GloMax Navigator (Promega). The control solvent (CON) used was DMSO.

TABLE 1

| target | siRNA sequence | | | | |
|---|---|---|---|---|---|
| (human) | SEQ | | SEQ | | |
| gene | NO: | sense (5′ to 3′) | NO: | antisense (5′ to 3′) | manufacturer |
| PPIA | 17 | CAAGAUGACUAAUGUCAAA | 18 | UUUGACAUUAGUCAUCUUG | Sigma-Aldrich |
| PPIB | 19 | GGUCUCUUCGGAAAGACUGUUCCA | 20 | UUGGAACAGUCUUUCCGAAGAGAC | invitrogen |
| | | A | | C | |
| PPIC | 21 | GCAACAGGAGAGAAAGGAUAUGGAU | 22 | AUCCAUAUCCUUUCUCUCCUGUUG | invitrogen |
| | | | | C | |
| PPID | 23 | GCCAAGUAAUUAAAGGAAUAGGAGU | 24 | ACUCCUAUUCCUUUAAUUACUUGG | invitrogen |
| | | | | C | |
| PPIE | 25 | CCAAUGAGAAUUAAGGAAGGCUCUU | 26 | AAGAGCCUUCCUUAAUUCUCAUUG | invitrogen |
| | | | | G | |
| PPIF | 27 | GGCAGAUGUCGUCCCAAAG | 28 | CUUUGGGACGACAUCUGCC | Sigma-Aldrich |
| PPIG | 29 | GGGAUAAGAGUGAGUUGAAUGAAA | 30 | AUUUCAUUCAACUCACUCUUAUCCC | invitrogen |
| | | U | | | |
| PPIH | 31 | CGUGGUGUUCUUUGAUGUCAGUAU | 32 | AAUACUGACAUCAAAGAACACGACG | invitrogen |
| | | U | | | |
| PPIL1 | 33 | GAACUUUGCUGAGUUGGCUCGUCG | 34 | UCGACGAGCCAACUCAGCAAAGUUC | invitrogen |
| | | A | | | |
| NKTR | 35 | GGAAAGCAGCAUGUCCGAAAGUAAA | 36 | UUUACUUUCGGACAUGCUGCUUUC | invitrogen |
| | | | | C | |
| PPWD1 | 37 | GACCCAACAAUAGUCUGUACAUCAU | 38 | AUGAUGUACAGACUAUUGUUGGGU | invitrogen |
| | | | | C | |

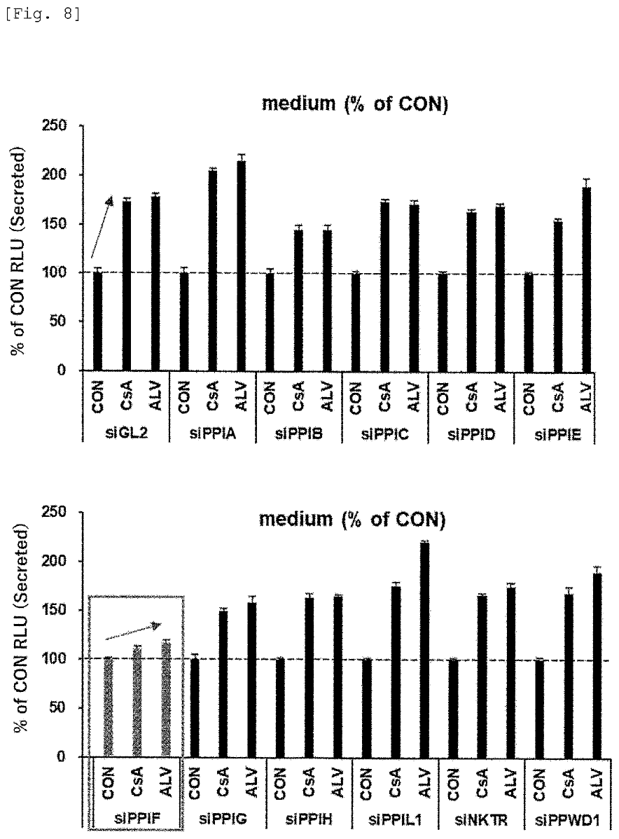

As a result, the effect of promoting the secretion of collagen trimers by CsA and Alisporivir decreased only in the cells knocked down with PPIF (cyclophilin D) (FIG. 8). Therefrom it was shown that CsA and Alisporivir promote the secretion of collagen trimers through binding to cyclophilin D.

### (Example 7) Influence of Cyclophilin D on Collagen Trimer Secretion

In the above-mentioned experiment of Example 4, it was confirmed that the promotion of collagen trimer secretion by CsA and Alisporivir is performed via cyclophilin D. Thus, an influence of cyclophilin D on collagen trimer secretion was investigated.

Using G1244D mutation-introduced HEK293T cells in the same manner as in Example 5, the collagen trimer secreting ability in cells in which various cyclophilins were knocked down with siRNA was investigated comprehensively.

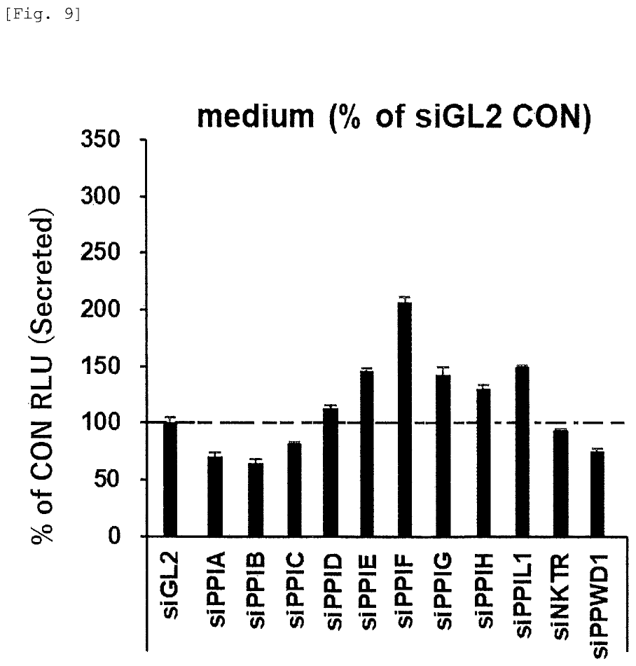

As a result, remarkable secretion of collagen trimers was confirmed in the cyclophilin D-knocked down cells (FIG. 9). Therefrom it was confirmed that inhibition of cyclophilin D promotes secretion of collagen trimers.

### (Example 8) Generation and Evaluation of AS Model Mouse

Since the existing AS model animal lacks the gene of collagen protein itself, evaluation of the pathological condition based on the dysfunction of collagen protein could not be performed. Now that the research achievements by the present inventors revealed that an important gene mutation of AS exists in Exon 41 of COL4A5, attempts were made to create a model mouse by which the pathological condition based on the dysfunction of collagen protein can be evaluated, by creating a G1244D or Exon41-deleted mutant mouse as a typical COL4A5 missense mutation (Glycine substitution).

In typical Alport syndrome patients and the existing model mice (Col4a3 KO, Col4a5-G5X), α345(IV) on GBM disappears. Since switching to α345(IV) after birth is not established, fragile α112(IV) in the fetal stage is persistently present in GBM. Furthermore, it has been reported that laminin—which is a basement membrane molecule—accumulates ectopically in GBM (Kashtan C E et al., J Am Soc Nephrol 2001; 12: 252-260.; Cosgrove D et al., Am J Pathol 2000; 157: 1649-1659). Thus, the expression of α5(IV), a2(IV), and Laminin was evaluated in the kidneys of the prepared G1244D mouse and ΔExon41 mouse.

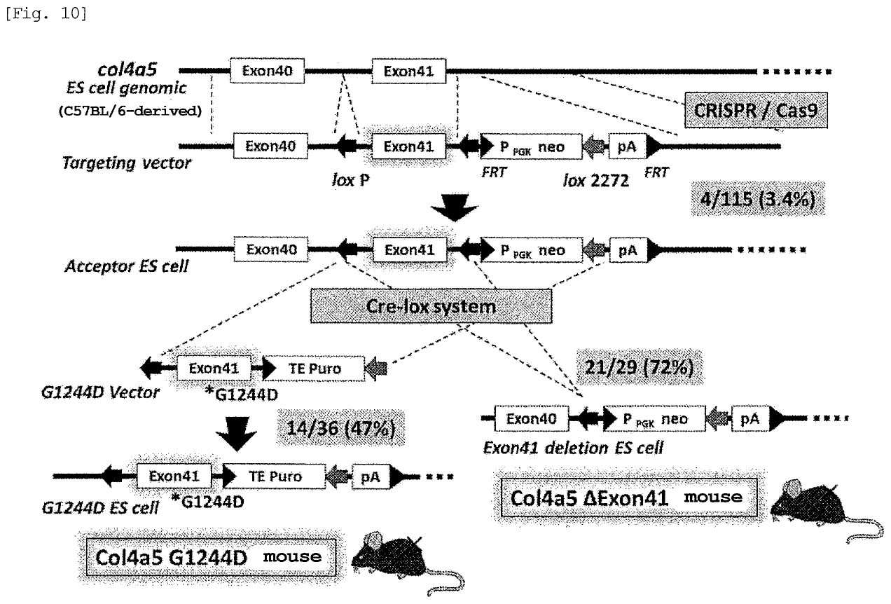

The kick-in method is a method developed by Prof. Kimi Araki et al. of the Institute of Resource Development and Analysis, Kumamoto University, and includes production of an acceptor ES cell in which the target exon into which the mutation is to be inserted is sandwiched between loxP sequences, followed by high probability recombination, using the Cre-loxP system, with the target exon having the target mutation (Tomonoh Y et al., PLoS One 2014; 9: e88549.). To generate a G1244D mutant mouse, a targeting vector into which Exon41 sandwiched between loxPs, a drug selection cassette, and homologous sequences before and after Exon41 are inserted was produced, and ES cells (acceptor ES cells) having a targeting sequence allele were constructed by homologous recombination using CRISPR/Cas9. Then, a vector having Exon41 with G1244D mutation, a drug selection cassette, and loxKMR3 and lox2272 on both ends of the cassette was produced, and the sequence between loxps were exchanged with that of the acceptor ES cells by recombination using Cre recombinase. Since loxKMR3 prevents re-cleavage after recombination by substituting 3 bases on the loxP3′ side, and recombination occurs only between lox2272 and lox2272 by substitution of the spacer sequence of loxP, the orientation of the inserted sequence can be specified. Finally, the drug selection cassette was removed in ES cells with the G1244D mutation, and Col4a5-G1244D mouse was established (FIG. 10). mRNA was extracted from the kidney of the produced mouse and the sequence of RT-PCR product was analyzed. As a result, it was confirmed that the desired mutation (G>A) was inserted in the G1244D mouse (not shown).

Acceptor ES cells prepared by the kick-in method have a sequence in which both ends of Exon 41 are sandwiched between loxPs. Thus, Exon41 was deleted by Cre recombinase in the acceptor ES cells, and the drug resistance cassette was removed to establish Col4a5-Exon41-deleted (ΔExon41) mouse (FIG. 10). When mRNA was extracted from the kidney of the produced mouse and the RT-PCR product was electrophoresed, a band shorter by 186 bp than Exon 41 was confirmed in ΔExon41 mouse (not shown). Furthermore, in sequence analysis, it was confirmed that Exon 41 was removed and a transcription product of continuous Exon 40 and Exon 42 was formed (not shown). In addition, even when Eon41 was deleted, the repeat of Glycine-X-Y in the collagen domain was maintained, suggesting that the ability of the α5(IV) mutant to form partial trimers was preserved. Specific experiment methods are described in the following.

### (1) Production of Targeting Vector

The kidney of C57BL/6 mouse (Charles river, 000664, Black6) was shredded, and genomic DNA was purified with DNeasy Blood & Tissue Kit (QIAGEN, 69504) and used as a template. A short arm of 1.9 kb (Arm1) on the 5′-side and 1.7 kb (Arm3) on the 3′-side were set around 0.9 kb (Arm2) including the target Exon 41. A deleted 3′-side region (147 bp) for inserting double-stranded break (DSB) was formed between Arm2 and Arm3. Arm1 and Arm2 were inserted into the both ends of loxP in the pBluescript II SK(+)loxP vector (Tomonoh Y et al., PLoS One 2014; 9: e88549) (vector 1). Arm3 was inserted into the downstream of the FRT-PGK-neo-lox2272-pA-FRT region of the p03 vector (Tomonoh et al., 2014, supra) (vector 2). The Arm1-loxP-Arm2 sequence of vector 1 was inserted before FRT of vector 2 to give a targeting vector.

### (2) Production of Mutation Vector

After insertion of Arm2 into the downstream of loxKMR3 in the pK-TEPS-2272 vector (Tomonoh et al., 2014, supra), G1244D mutation was inserted. The sequence of each lox is as follows.

| (SEQ ID NO: 39) | |
|---|---|
| loxP: | ATAACTTCGTATAGCATACATTATACGAAGTTAT |
| (SEQ ID NO: 40) | |
| loxKMR3: | ATAACTTCGTATAGCATACATTATACCTTGTTAT |
| (SEQ ID NO: 41) | |
| lox2272: | ATAACTTCGTATAGGATACTTTATACGAAGTTAT |

(3) Gene Transfer into ES Cells

The produced targeting vector was purified by phenol/chloroform extraction, washed with 70% ethanol, dehydrated with 100% ethanol, dissolved in sterilized TE, and then 40 μg of the vector was used for one electroporation. ES cells (6NK) cultured in a 10 cm petri dish for culture to a confluent state were washed with PBS, and the cells were detached with trypsin and suspended in a medium. After centrifuging the cell solution (4° C., 800 rpm), the supernatant was removed and the pellets were resuspended in cold PBS. The targeting vector and Cas9 vector were mixed with the cell solution and transfected by electroporation. Thereafter, the cells were incubated at room temperature for 10 min, suspended in a medium, seeded in a 10 cm petri dish for culture, and statically cultured under the conditions of 5% CO₂, 37° C. After 24 hr, the medium was replaced with a medium containing G418 (Nacalai, 16548) (200 μg/mL), and the drug selection was performed over a period of about 1 week. Colonies were picked up, proliferated, and sequence insertion was confirmed by PCR and Southern blotting. Electroporation of the mutation vector and Cre expression vector was similarly performed on the clone in which the target sequence was confirmed, and the drug was selected over a period of about 1 week in a medium containing Puromycin (Nacalai, 14861-84) (2 mg/ml). Colonies were picked up, proliferated, and the sequence was confirmed by PCR.

### (4) Generation of Mutant Mouse

Mice were generated from ES cell clones having each target sequence. The mouse 2-cell phase of ICR lineage was immersed in an ES cell suspension, incubated overnight, and then the chimeric embryo was transplanted into the uterus of a pseudopregnant female. The obtained littermate chimera was mated with C57BL/6J mouse to obtain F1 mouse. Furthermore, the drug resistance cassette of FRT was removed by FLP recombinase, and the mouse was established.

### (5) Genotyping

Tail was collected from mouse and washed with 70% ethanol. After air drying, 50 mM NaOH was added, the tail was shredded, boiled for 10 min, and then neutralized with 1M-Tris-HCl. After centrifugation (15,000 rpm, 4° C., 10 min), the supernatant was extracted and used as a DNA sample. PCR reaction (94° C.-2 min: 1 cycle, 98° C.-10 sec-68° C.-1 min/kb: 20 cycle, 4° C.-hold) of each sample was performed, and the band was confirmed by agarose gel electrophoresis. The sequences of the primers used for each mouse genotyping are shown in Table 2.

### (6) Extraction of RNA

The kidney tissue was homogenized after adding 1 mL of RNAisoPlus (TaKaRa, 9109) to a 1.5 mL tube, and shredding the tissue. Then, 0.2 mL of chloroform was added, and the mixture was stirred well and allowed to stand at room temperature for 2 min. After centrifugation (12,000 rpm, 4° C., 15 min), the aqueous layer was transferred to a new 1.5 mL tube, an equal amount of chloroform was added, and the mixture was stirred and then centrifuged again (12,000 rpm, 4° C., 15 min). The aqueous layer was further transferred to a new 1.5 mL tube, an equal amount of isopropanol was added, and the mixture was stirred and then centrifuged (12,000 rpm, 4° C., 30 min). The obtained pellets were washed twice with 70% ethanol, air dried, and then dissolved in DEPC-treated water. The yield and purity of the obtained total RNA were measured using an Epoch Microplate Spectrophotometer (BioTek). After confirming that it was a high-purity RNA with an OD260/OD280 ratio of not less than 2.0, the RNA was used in various experiments.

### (7) Semi-RT-PCR Method

A reverse transcriptase reaction (37° C.-30 min, 85° C.-10 sec) was performed using Prime Script (registered trade mark) RT reagent kit (TaKaRa, RR036A) and the total RNA extracted from the kidney as a template. Thereafter, PCR reaction (94° C.-2 min: 1 cycle, 98° C.-10 sec-68° C.-1 min/kb: 20 cycle, 4° C.-hold) was performed using KOD Plus (TaKaRa), and the band was confirmed by agarose gel electrophoresis. The base sequence of the PCR product was confirmed by the cycle sequence method (Sigma-Aldrich JAPAN). The sequences of the primers used in the PCR reaction are shown in Table 2.

TABLE 2
Table 2. Primers used in genotyping and Semi-RT-PCR

| | | | | SEQ |
|---|---|---|---|---|
| | | | 5′ to 3′ sequence | ID NO: |
| Mouse for | Control | Arm2 check Fw | aagggttccggatccccatcaagcttatcg | 42 |
| genotyping | | Arm3 check Rv | gggtagggatggaagttcaccaaagccc | 43 |
| | | Deletion arm2-3 Fw | cctgtgctcactctttagtagatcaattttgcatcc | 44 |
| | G1244D | Arm1 nick Fw | gctacacataatgacatacacctgaggctg | 45 |
| | | Recon check Rv | ccgagctcgtcgacaagctcgaattagcttgg | 46 |
| | | Deletion arm2-3 Rv | gagtcaatttgaccatgctgtgctgctgaag | 47 |
| | ΔExon41 | Outside arm1 Fw | ggaggtgacatgtgccagtctgcgggaactgg | 48 |
| | | Deletion arm2-3 Rv | gagtcaatttgaccatgctgtgctgctgaag | 49 |
| | | FRT-arm3 check Rv | acacagatctgatatcatcgatgaattcgagcttg | 50 |
| | | | c | |

| | | | |
|---|---|---|---|
| Semi RT-PCR | Col4a5 Exon37 | ggtcggtgggagagactggtcttcctg | 51 |
| | Col4a5 Exon46 | tggagcctggtactccaatgggtcc | 52 |

### (8) Preparation of Frozen Section of Kidney Tissue

For the mouse kidney frozen block, a fresh mouse kidney after dissection was cut in half, immersed in OCT compound (Sakura Finetech) in Cryomold, and rapidly frozen in liquid nitrogen. The kidney frozen block was sliced in a thickness of 5 μm using a cryostat (Leica) and used as a frozen section. The prepared sections were dried and stored at −80° C. until staining.

### (9) Immunofluorescence Staining

The kidney frozen section was fixed by immersing in ice-cooled acetone solution at −20° C. for 5 min. Thereafter, it was blocked for 1 hr at room temperature by Serum free protein block (DAKO, X0909) in a moistening box. After washing with PBS, the primary antibody diluted 1:100 with Antibody diluent (DAKO, 50809) was reacted at room temperature for 1 hr. As the primary antibody, anti-Laminin polyclonal antibody (sigma, L9393), Anti-Human Alpha5(IV) Antibody, Clone H53 (Chondrex, 7078), or Anti-Human Alpha2(IV) Antibody, Clone H22 (Chondrex, 7071) was used. Thereafter, washing with PBS was performed three times, and a fluorescently-labeled secondary antibody (Alexa fluor antibody) diluted 1:500 with Antibody diluent (DAKO) was reacted at room temperature for 1 hr. Washing with PBS was performed three times, the section was mounted in Vector shield (Vector, H-1000), and imaging and analysis was performed using BZ-X700 (Keyence).

### (10) Collection of Urine

Urine was collected by 24 hr urine pooling using a mouse metabolism cage (AS ONE). The collected urine was centrifuged (12,000 rpm, 4° C., 5 min) to remove contaminants, and the supernatant was collected and stored at −80° C.

### (11) Measurement of Proteinuria Score

The proteinuria score was calculated by the ratio of urinary total protein concentration/urinary creatinine concentration. The total urinary protein concentration was measured using the Bradford method. The urinary creatinine level was measured according to the protocol and using the creatinine measurement kit (WAKO).

### (12) CBB Staining

Urine samples were prepared with 5× sample buffer for reduction (0.25 M Tris-HCl (pH 6.8), 10% SDS, 4.1M β-mercaptoethanol, 50% glycerol, BPB). SDS-PAGE was performed using 12% polyacrylamide gel and the band was detected by staining with CBB (Coomassie Brilliant Blue).

### (13) Results

G1244D mouse showed the same pattern of α5(IV) expression on the basement membrane as the control mouse, and no change was observed in the expression of α2(IV) and laminin on the mesangial matrix (not shown). In addition, the kidney function was evaluated. As a result, G1244D mouse did not show proteinuria even after 18 weeks of age when the existing Col4a5-G5X mouse showed remarkable pathological conditions, and even at 35 weeks of age, no change was observed in urinary albumin leakage (not shown).

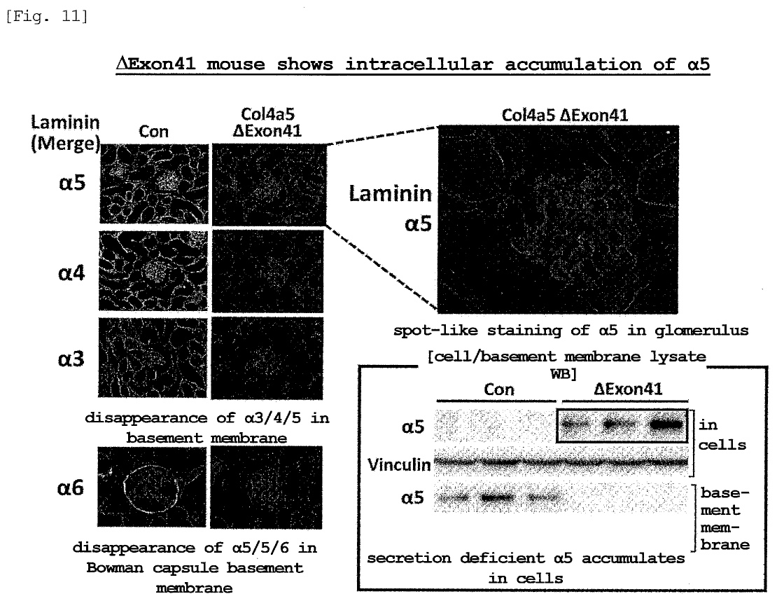

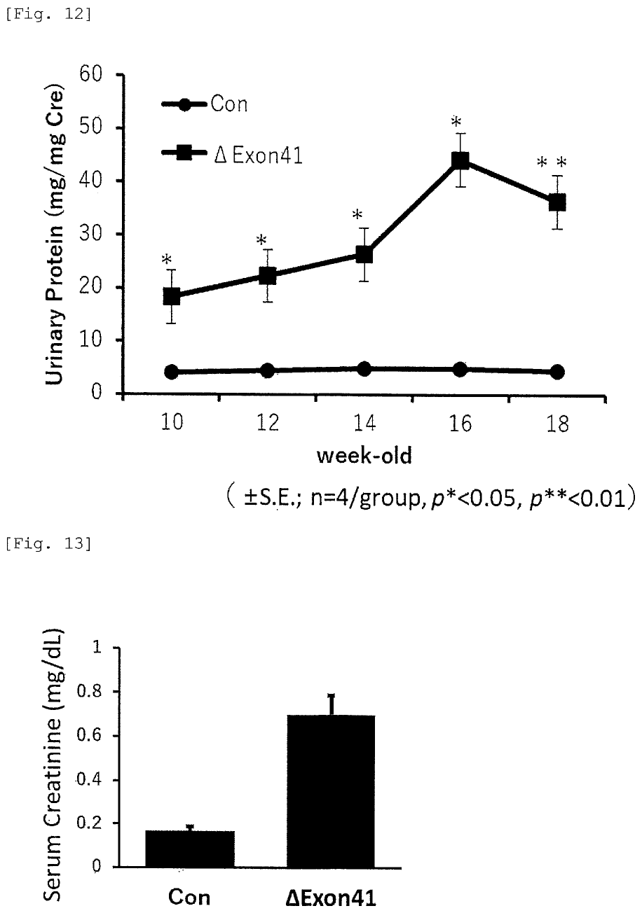

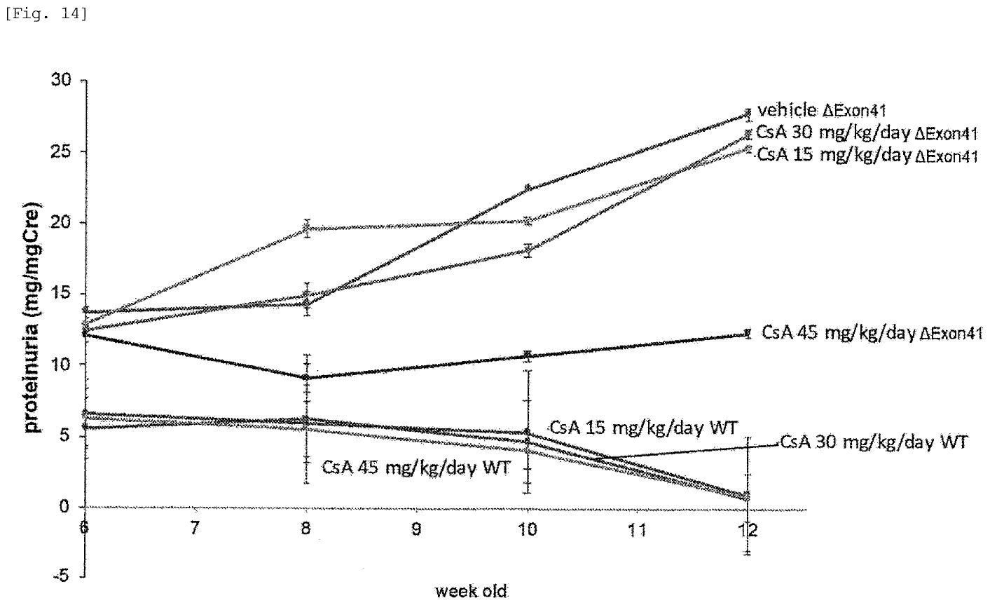

On the other hand, α5(IV) expression on the basement membrane disappeared in ΔExon41 mouse, and α2(IV) was widely expressed instead in GBM. In addition, consistent with previous reports, it was clarified that laminin, which is mainly present as a mesangial matrix in normal glomerulus, shows an increase in ectopic expression in GBM of ΔExon41 mouse (FIG. 11). Furthermore, the kidney function was evaluated. As a result, ΔExon41 mouse exhibited progressive proteinuria (FIG. 12), and significant leakage of urinary albumin and the like already at 10 weeks of age (not shown). In addition, the serum creatinine level increased (FIG. 13). Therefore, ΔExon41 mouse does not undergo normal GBM formation due to abnormal trimer formation of α5(IV) variant, and shows progressive renal pathology such as leakage of albumin and protein into urine. It was therefore shown that the mouse is a model mouse that reflects the dysfunction of AS.

### (Example 9) Administration of Cyclosporine a to Alport Syndrome Model Mouse

Whether the AS-like pathology in the ΔExon41 mouse produced in Example 7 is improved by the administration of cyclosporine A was confirmed.

### (1) Administration of Cyclosporin A

Cyclosporin A powder was weighed and dissolved in ethanol, and then adjusted to 15, 30, 45 mg/kg/day using 0.5% methylcellulose. After measuring the body weight, the adjusted cyclosporin A was administered intraperitoneally to the mouse once per day. 0.5% Methylcellulose added with ethanol to the final 4% was administered to the control group.

### (2) Collection of Urine

Urine was collected once every two weeks from the age of 6 weeks using a metabolic cage (AS ONE) in which mice can freely ingest food and water and can move. The urine accumulated in 24 hr was suspended well and 1 mL was collected in a 1.5 mL tube. The urine was centrifuged (4° C., 12,000 rpm, 5 min) to remove contaminants in the collected urine, and the supernatant was stored at −80° C. as a urine sample.

### (3) Calculation of Proteinuria Score

The proteinuria score was calculated by the ratio of urinary total protein concentration/urinary creatinine concentration. The total urinary protein concentration was measured using the Bradford method. The urinary creatinine concentration was measured according to the protocol and using the creatinine measurement kit.

As a result, it was shown that proteinuria in ΔExon41 mouse was improved by the administration of cyclosporine A at 45 mg/kg/day (FIG. 14). Therefore, it was shown that this model mouse can be used for the evaluation of AS treatment effect via cyclophilin D.

1. A method for promoting secretion of a collagen trimer in a cell having a type-IV collagen gene having a mutation, which method comprises treating the cell with a cyclophilin D inhibitor excluding cyclosporine A.

2. The method according to claim 1, wherein the cyclophilin D inhibitor is a cyclosporine derivative represented by the following formula, or a salt thereof, or a hydrate or a solvate thereof: 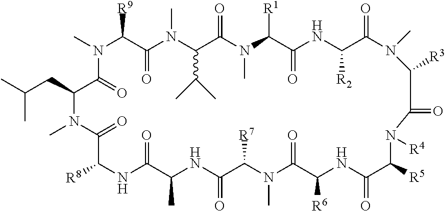
wherein
R¹ is a C5-9 alkyl group or C5-9 alkenyl group substituted by a hydroxyl group; or a group represented by the following formula 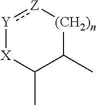
wherein
X is O or NR¹¹
R¹¹ is H or a C1-6 alkyl group,
Y is CR¹²R¹³, CR¹⁴ or >C═O,
R¹² is H, a C1-6 alkyl group, or an OH group,
R¹³ is H, a 01-6 alkyl group, or an OH group,
R¹⁴ is H or a C1-6 alkyl group,
Z is (CH₂)ₘ, CR¹⁵, NR¹⁶, or O,
R¹⁵ is H or a C1-6 alkyl group,
R¹⁶ is H or a C1-6 alkyl group,
m is an integer of 1-3,
n is 0 or 1, and
a bond between Y and Z shown by a solid line and a broken line is a single bond or a double bond, when the bond between Y and Z is a single bond, Y is CR¹²R¹³ or C═O, and Z is (CH₂)ₘ, NR¹⁶, or O; when the bond between Y and Z is a double bond, Y is CR¹⁴, and Z is CR¹⁵,
R² is a C1-4 alkyl group optionally substituted by a hydroxyl group,
R³ is a hydrogen atom; a C1-6 alkyl group, a 01-6 alkoxy group, or a 01-6 alkylthio group, each optionally substituted by a hydroxyl group and/or a halogen atom; or a group selected from the following formulas: 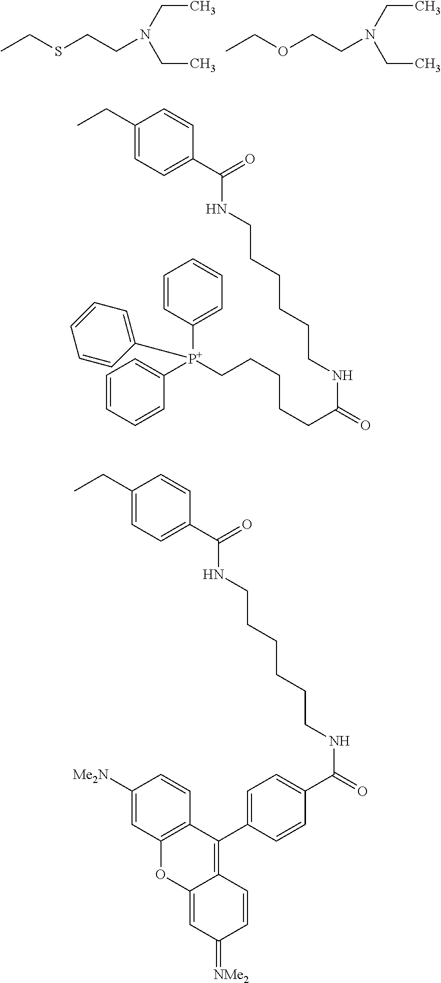 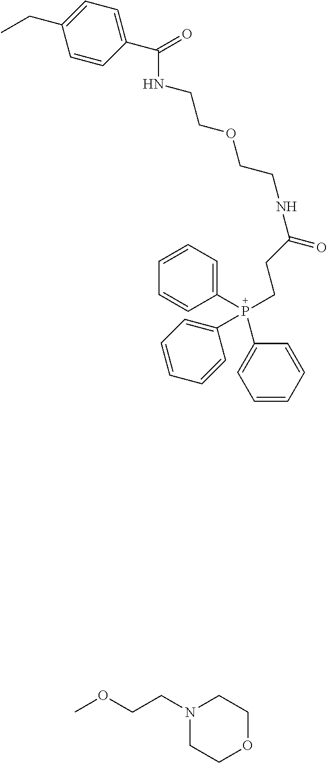
R⁴ is a C1-4 alkyl group,
R⁵ is a C1-6 alkyl group optionally substituted by a hydroxyl group and/or a C1-6 alkoxy group, or a group represented by the following: 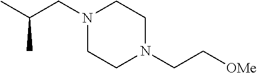
R⁶ is an n-propyl group or an isopropyl group,
R⁷ is a C1-4 alkyl group,
R⁸ is a C1-4 alkyl group optionally substituted by a hydroxyl group,
R⁹ is a branched C3-4 alkyl group, and
a bond shown by a wavy line means that a steric structure of a chiral carbon atom to be bonded is an R form, an S form, or a mixture thereof.

3. The method according to claim 2, wherein the cyclosporine derivative is a compound selected from the following:

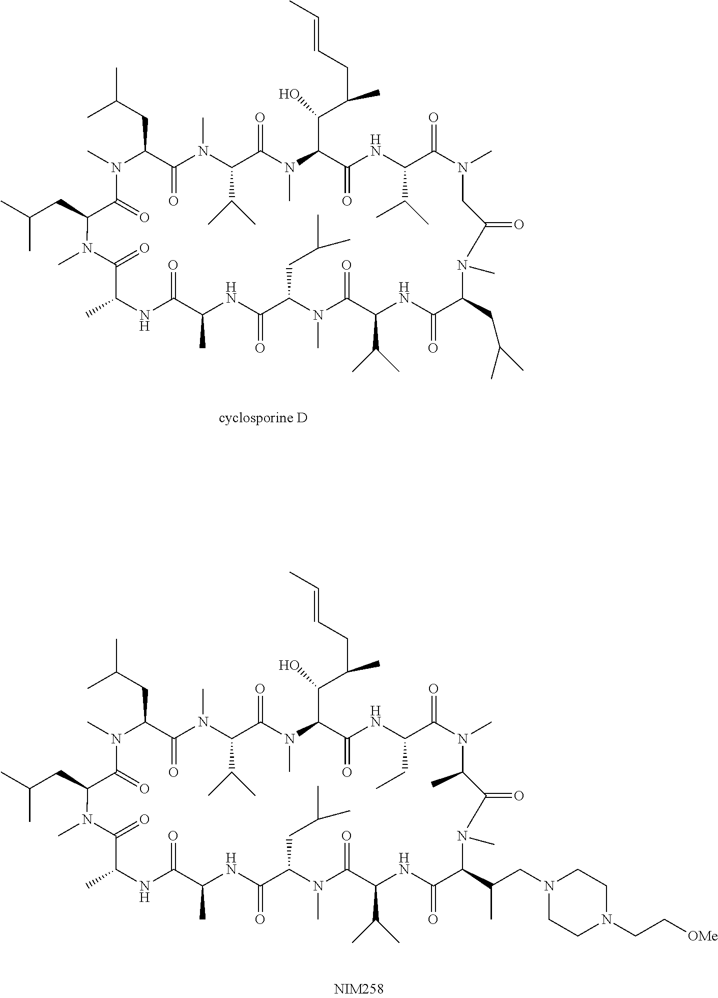

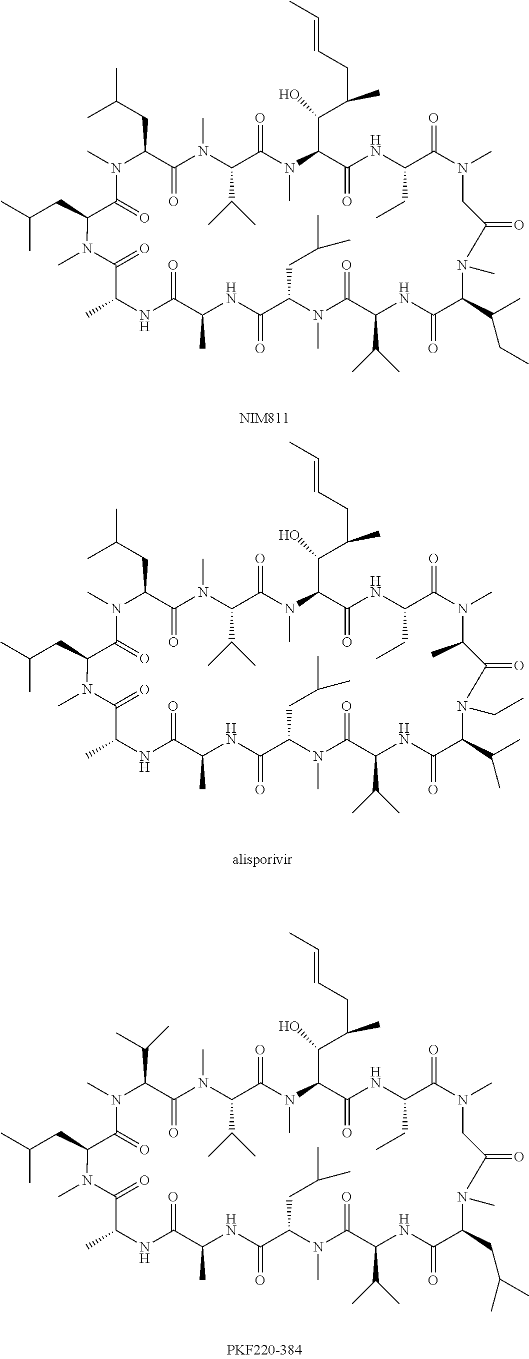

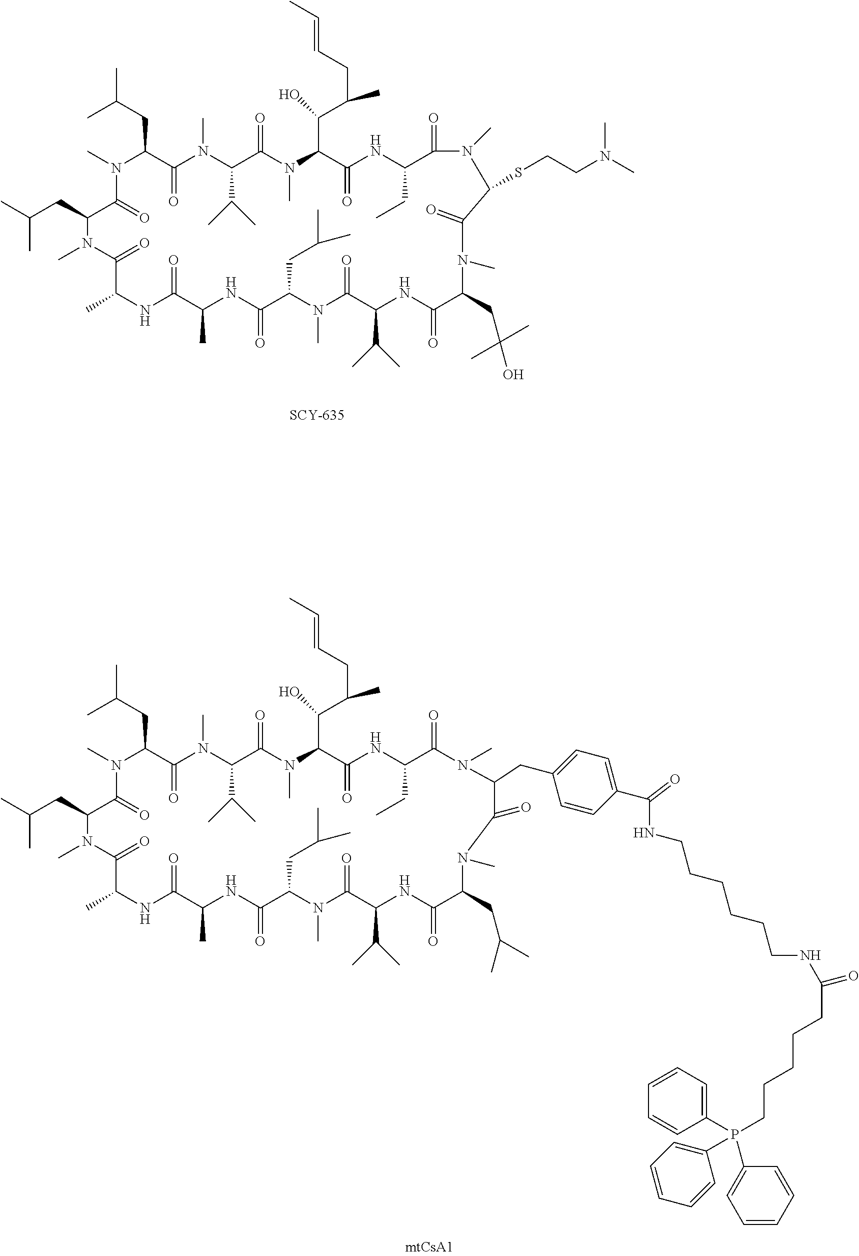

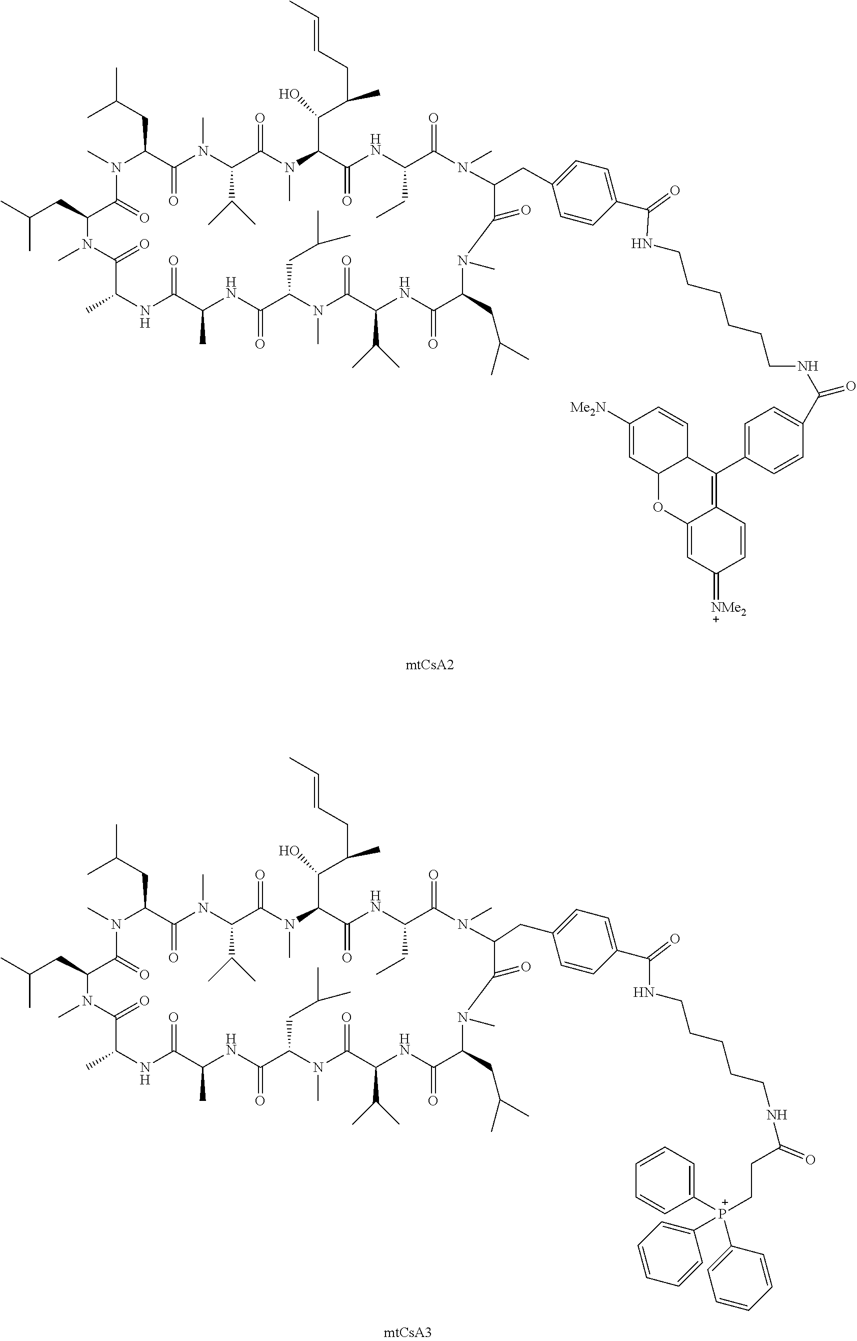

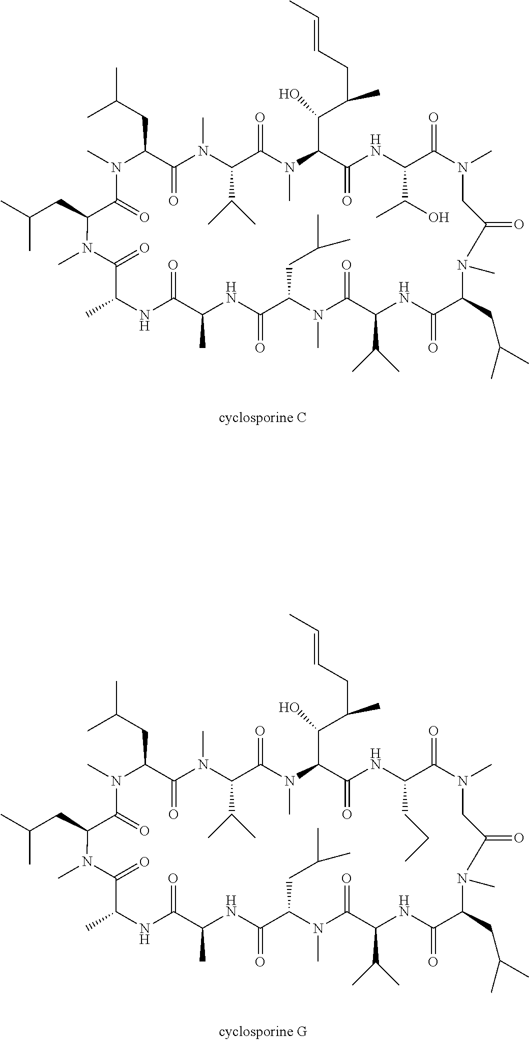

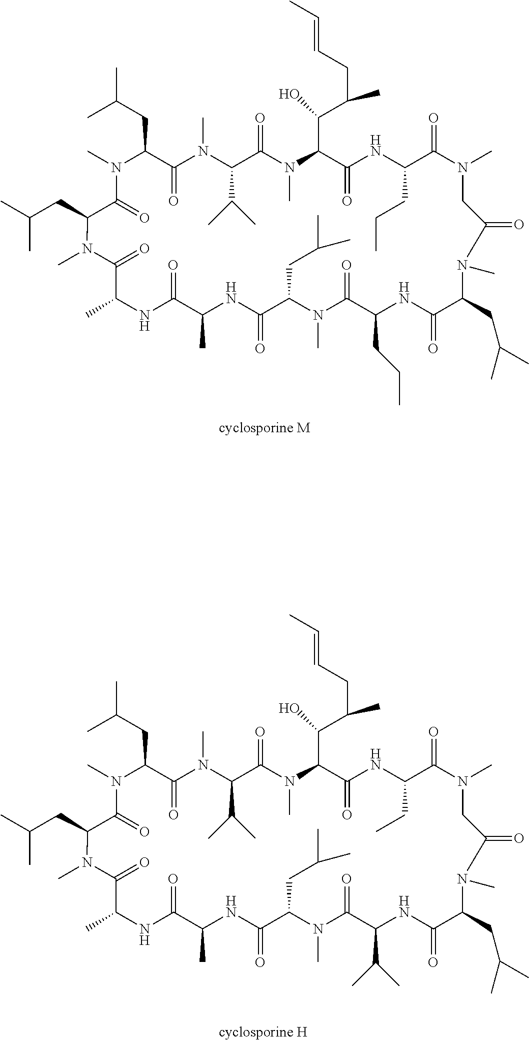

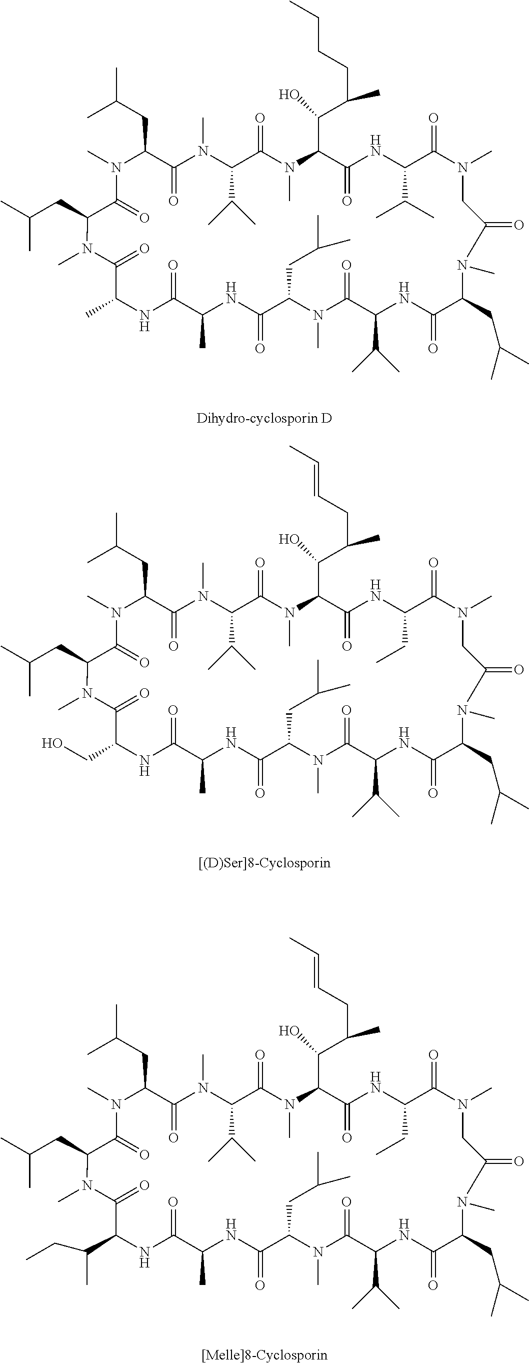

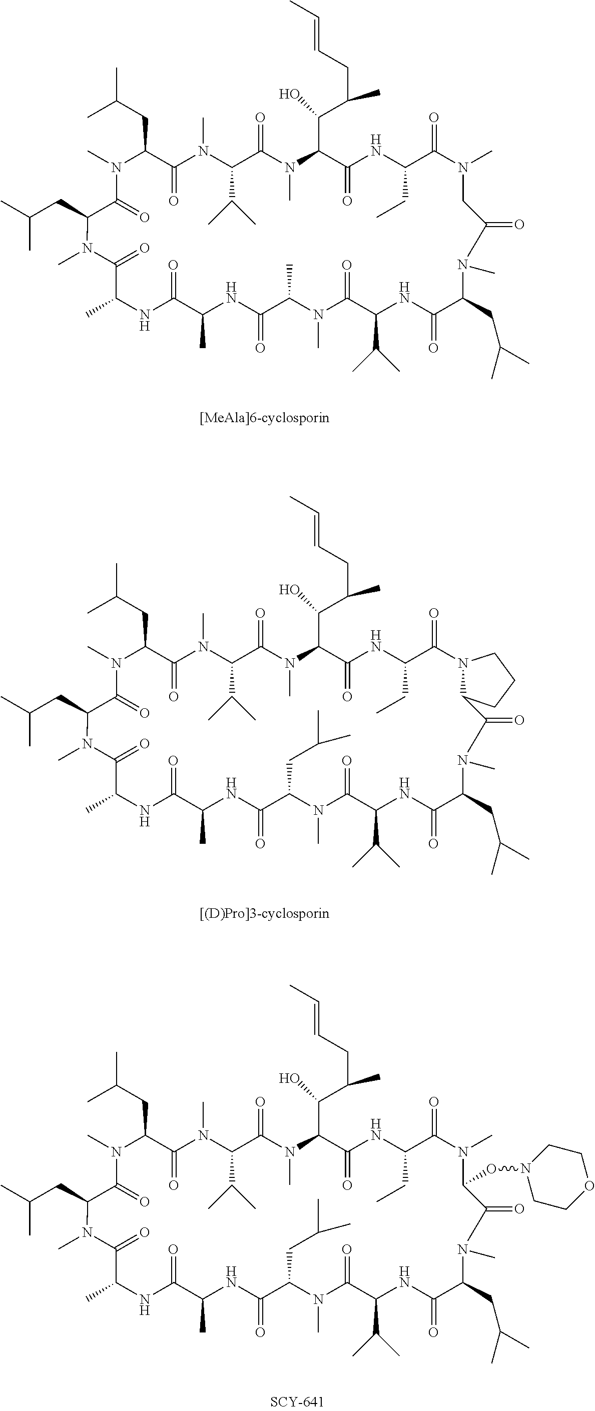

4. The method according to claim 1, wherein the cyclophilin D inhibitor does not show a calcineurin inhibitory activity.

5. The method according to claim 1, wherein the cyclophilin D inhibitor is Alisporivir or NIM258.

6. The method according to claim 1, wherein the cyclophilin D inhibitor is siRNA, shRNA, miRNA against cyclophilin D, an anti-cyclophilin antibody, an aptamer against cyclophilin D, or an antisense nucleic acid molecule against cyclophilin D.

7. The method according to claim 1, wherein the type-IV collagen gene having a mutation is COL4A3, COL4A4, or COL4A5.

8. The method according to claim 7, wherein the type-IV collagen gene having a mutation is COL4A5.

9. The method according to claim 1, wherein the mutation in the type-IV collagen gene causes Alport syndrome.

10. The method according to claim 8, wherein the mutation in the type-IV collagen gene is a mutation that reduces the secretion of collagen trimer in COL4A5.

11. The method according to claim 8, wherein the mutation in the type-IV collagen gene is G1030S, G1107R, or a mutation in a region of Exon41 in COL4A5.

12. The method according to claim 11, wherein the mutation in a region of Exon41 in COL4A5 is G1220D, G1241C, G1241V, or G1244D.

13. A method for treating, preventing, or improving Alport syndrome, comprising administering an effective amount of a cyclophilin D inhibitor excluding cyclosporin A to a subject in need thereof.

14. The method according to claim 13, wherein the subject is a patient judged to have a mutation that decreases secretion of collagen trimer in COL4A5.

15. The method according to claim 14, wherein the mutation that decreases secretion of collagen trimer is G1030S, G1107R, or a mutation in a region of Exon41 in COL4A5.

16. The method according to claim 15, wherein the mutation in the region of Exon41 in COL4A5 is G1220D, G1241C, G1241V, or G1244D.

17. A non-human animal model having a G1220D mutation in the COL4A5 gene, or a non-human animal model having deletion of Exon41 of the COL4A5 gene.

18. The non-human animal model according to claim 17, wherein the animal is a mouse.

19. A method for evaluating a therapeutic effect of a test drug on Alport syndrome, comprising
administering the test drug to the non-human animal model according to claim 17,
observing or measuring the Alport syndrome or a level to be an index thereof in the non-human animal model, and
when the administration of the test drug improves the symptoms or conditions based on the deficiency in the secretion of collagen trimers, or levels to be the indices thereof, determining that the test drug may have a therapeutic effect on the Alport syndrome.

20. The method according to claim 19, wherein the symptoms or conditions based on the deficiency in the secretion of collagen trimers, or levels to be the indices thereof in the non-human animal model is at least one or more kinds selected from the following:
(i) disappearance of α5(IV) expression on the basement membrane
(ii) widespread expression of α2(IV) in GBM
(iii) increased ectopic expression of laminin
(iv) proteinurina
(v) leakage of urinary albumin
(vi) elevated serum creatinine levels
(vii) progressive renal condition.
